(19) United States
(12) Patent Application Publication
(10) Pub. No.: US 2006/0286602 A1
(43) Pub. Date: Dec. 21, 2006

(54) Method and markers for the diagnosis of renal diseases

(76) Inventors: Harald Mischak, Sehnde (DE); Thorsten Kaiser, Hannover (DE); Stefan Wittke, Hannover (DE); Michael Walden, Hannover (DE)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/126,385

(22) Filed: May 10, 2005

Related U.S. Application Data
(60) Provisional application No. 60/569,230, filed on May 10, 2004.

(51) Int. Cl. G01N 33/53

(52) U.S. Cl. 435/7.1; 435/7.92; 436/86

(57) ABSTRACT

Certain embodiments of the invention relate to means and methods for the diagnosis of a renal disease, particularly to differential diagnosis. Renal diseases of particular interest in the context of the invention are IgA-nephropathy, membranous glomerulonephritis (MGN), minimal-change-disease (MCD), focal segemental glomerulosclerosis (FSGS), and diabetic nephropathy. Particularly, the method comprises (a) measuring the presence or the absence of a polypeptide marker in a urine sample, wherein the polypeptide marker is selected from the group of polypeptide markers shown in tables 1 to 22, and (b) comparing the probability of the presence of this marker in a disease patient to the probability of the presence of this marker in a control patient, wherein the individual probabilities are as indicated in the tables, and wherein (c1) if the probability of the presence of this marker in a disease patient is higher than the probability of the presence of this marker in a control patient, the presence of this marker is indicative for a higher probability of having the disease, or (c2) if the probability of the presence of this marker in a disease patient is lower than the probability of the presence of this marker in a control patient, the absence of the marker is indicative for a higher probability of having the disease.

### RELATED APPLICATION

The present application claims the benefit of U.S. Provisional Application No. 60/569,230 filed May 10, 2004, which is hereby incorporated herein in its entirety by reference.

### TECHNICAL FIELD OF THE INVENTION

The present invention relates to the diagnosis, particularly differential diagnosis, of renal diseases.

### BACKGROUND

The number of patients presenting with renal diseases has been increasing in the recent years. Thus, renal diseases present an increasing problem to the health system. Many renal diseases are irreversible, therefore an early diagnosis and/or a differential diagnosis of renal diseases is important. Early diagnosis and a therapy precisely tailored to each particular disease could reduce the number of patients requiring dialysis and could also reduce the high cardiovascular risk of the patients.

### SUMMARY AND DETAILED DESCRIPTION OF THE INVENTION

Currently, precise diagnosis and/or differential diagnosis relies mostly on kidney biopsies. Although biopsies serve as the current “gold standard” in renal diagnostics, biopsies have the disadvantage of being invasive and therefore being conducted only on selected patients.

Urine analysis is a different approach to diagnose renal diseases. However, currently only few parameters of urine are routinely measured, for example creatinin, urea, albumin, blood cells (such as leukocytes and erythrocytes), bacteria, sugar, urobilinogen, bilirubin and pH value. The diagnostic value of these analyses is limited, as they lack sufficient sensitivity and/or selectivity, particularly for differential diagnosis.

Several attempts have been made to analyze the proteins contained in urine.

V. Thongboonkerd et al. have used two-dimensional polyacrylamide gel electrophoresis (2D-PAGE) in combination with matrix-assisted laser desorption ionization-time-of-flight (MALDI-TOF) mass spectrometry followed by mass fingerprinting to investigate normal human urinary proteins. A total of 67 protein forms of 47 unique proteins was identified (V. Thongboonkerd et al. (2001). Proteomic analysis of normal human urinary proteins isolated by acetone precipitation or ultracentrifugation. Kidney International, vol. 62, p. 1461-1469).

C. S. Spahr et al. have digested the proteins contained in urine samples with trypsin and identified 751 peptides from 124 proteins by means of liquid chromatography-tandem mass spectrometry (C. S. Spahr et al. 2001). Towards defining the urinary proteome using liquid chromatography-tandem mass spectrometry. I. Profiling an unfractionated tryptic digest. Proteomics vol. 1, p. 93-107).

These studies relate only to healthy individuals. The studies have not addressed the question whether alterations in presence of urinary polypeptides can be used for diagnosis or differential diagnosis of renal diseases.

It has been proposed to use the presence or absence of polypeptides in urine for the diagnosis of membranous glomerulonephritis (MGN) (von Neuhoff et al. (2004). Mass Spectrometry for the Detection of Differentially Expressed Proteins: A Comparison of Surface-Enhanced Laser Desorption/Ionization and Capillary Electrophoresis Mass Spectrometry. Rapid Communications in Mass Spectrometry, vol. 18: 149-156). However, samples of only 8 patients were used in the study, which was mainly concerned with the comparison of different analysis methods. The actual diagnostic value of the markers has remained unclear.

Consequently, there is need for a fast and simple methods and means for diagnosis, particularly differential diagnosis, of renal diseases.

Accordingly, an object of certain embodiments of the invention is to provide methods and means for the diagnosis of renal diseases, particularly for differential diagnosis of renal diseases. It is a particular object of certain embodiments of the invention to provide methods and means for the diagnosis and/or differential diagnosis of IgA-nephropathy, which is the most common glomerulopathy.

According to a first aspect of the present invention, the problem is solved by the use of the presence of at least one polypeptide marker in a urine sample for the diagnosis, preferably the differential diagnosis, of a renal disease, wherein the polypeptide marker is selected from the group of polypeptide markers as shown in table 1 to 22.

In the context of the present invention, it has been found that with the help of the polypeptide markers as shown in table 1 to 22 it is possible to reliably diagnose or differentially diagnose, respectively, different renal diseases.

The present invention has numerous advantages compared to the state of the art. First, the presence of the polypeptide markers according to the invention can be determined in urine samples. Therefore, there is no need to take biopsies. Thus, the present invention allows a simplified and fast diagnosis of renal diseases, allowing to screen patients regularly for the presence of renal diseases and to diagnose renal diseases at early stages. Furthermore, the polypeptide markers according to the invention can be used for differential diagnosis between different renal diseases. The high number of markers identified according to the present invention allows to increase both specificity and sensitivity of diagnosis as compared to the use of only a single or a small number of markers. Also, the present invention provides methods which allow to measure said polypeptide markers without the use of specific ligands such as antibodies or aptamers.

The polypeptide markers as shown in the tables have been identified by a method named capillary electrophoresis-mass spectrometry (CE-MS), which will be described further below. Furthermore, the method has been described in detail in von Neuhoff et al. (2004) (Mass Spectrometry for the Detection of Differentially Expressed Proteins: A Comparision of Surface-Enhanced Laser Desorption/Ionization and Capillary Electrophoresis/Mass Spectrometry. Rapid Communications in Mass Spectrometry, vol. 18: 149-156). Starting from the parameters defining the polypeptide markers, it is possible by methods known in the art to identify the sequence of the corresponding polypeptides and then to synthesize or produce the corresponding polypeptides, e.g. with the help of protein synthesis or expression of the corresponding gene in appropriate cells.

The markers are defined by there mass and their migration time in capillary electrophoresis (CE), particularly mass and their migration time obtained according to Example I. It is known that CE migration times can vary, typically in the range of 5 min, more typically in the range of 3 minutes. However, the sequence of markers being eluted is typically the same or very similar for each CE system applied. The system can be calibrated by use of polypeptides which are present in almost any urine sample, e.g. by the polypeptides given in tables 23 or 24. Furthermore, the polypeptides given in SEQ ID NO: 1 to SEQ ID NO: 5 can serve for calibration.

Variation of the masses between measurements or between different mass spectrometers is relatively small, typically it is in the range of plus or minus 0.05%.

In table I, polypeptide markers are listed which are preferred for the discrimination between healthy individuals and individuals suffering from a renal disease, particularly from a glomerulonephritis or glomerulopathy.

In table 2, polypeptide markers are listed, which are preferred for a discrimination between FSGS and the healthy condition.

In table 3, polypeptide markers are listed, which can be used for differential diagnosis between FSGS and MCD.

In table 4, polypeptide markers are listed, which are preferred for a differential diagnosis of FSGS and MGN.

In table 5, polypeptide markers are listed, which are preferred for a differential diagnosis between FSGS on the one hand, and MCD or MGN on the other hand.

In table 6, polypeptide markers are listed, which are preferred for diagnosis of MCD as compared to the healthy condition.

In table 7, polypeptide markers are listed, which are preferred for differential diagnosis between MCD and MGN.

In table 8, polypeptide markers are listed, which are preferred for differential diagnosis between MCD on the one hand, and FSGS or MGN on the other hand.

In table 9, polypeptide markers are listed, which are preferred for diagnosis of MGN as compared to the healthy condition.

In table 10, polypeptide markers are listed, which are preferred for differential diagnosis between MGN on the one hand, and FSGS or MCD on the other hand.

In table 11, polypeptide markers are listed, which are preferred for diagnosis of IgA-nephropathy or MGN on the one hand as compared to the healthy condition.

In table 12, polypeptide markers are listed, which are preferred for diagnosis of IgA-nephropathy as compared to the healthy condition.

In table 13, polypeptide markers are listed, which are preferred for differential diagnosis between IgA-nephropathy and MGN.

In table 14, polypeptides are listed with their respective frequency in healthy, FSGS, MCD, and MGN patients.

In table 15, polypeptides are listed which have been used for differential diagnosis between healthy individuals and renal patients using support vector machines according to Example 1.

In table 16, polypeptides are listed which have been used for differential diagnosis between healthy, FSGS, MCD, and MGN patients using random forest analysis according to Example 1.

In table 17, polypeptides are listed which have been used for differential diagnosis between MCD and MGN patients using support vector machines according to Example 1.

In table 18, polypeptides are listed which have been used for differential diagnosis between MCD and FSGS patients using support vector machines according to Example 1.

In table 19, polypeptides are listed which have been used for differential diagnosis between MGN and FSGS patients using support vector machines according to Example 1.

In table 20 and 21, polypeptides are listed which have been identified in von Neuhoff et al. (2004), which has been cited above.

In table 22, polypeptides are listed which can be used for diagnosis of diabetes and/or diabetic nephropathy.

In table 23, polypeptides are listed, which are preferred as internal standards to standardize the CE-time.

In table 24, polypeptides are listed, which are preferred as internal standards to standardize the CE-time if the pressure method (0.3 to 1 psi) according to Example 1 is used. These standards are e.g. preferred as internal standards in diagnosis of IgA-nephropathy.

In table 25, clinical data of renal patients are listed whose samples were used for identification of polypeptide markers according to Example 1. Abbreviations: CsA, Cyclosporin A; PS, prednisolone; +, frequent relapse; −, currently no immunosuppression; *, clinically unclear whether MCD or FSGS.

The polypeptide markers used according to the present invention can be identified and their presence can be measured in urine samples. Urine samples can be taken as known in the state of the art. Preferably, midstream urine is used in the context of the present invention.

The polypeptide markers used according to the present invention can be gene expression products such as proteins, peptides, and fragments or other degradation products of proteins or peptides. They can be modified by posttranslational modifications, e.g. by glycosylation, phoshorylation, alkylation or disulfide bond. It is known that fragments and degradation products can have a different diagnostic value and/or physiological role than the protein or peptide they have been derived from. For example, in different diseases, different proteolytic degradation products or fragments can be found. It is also considered to be within the scope of the present invention if the urine sample is pretreated to chemically modify the polypeptide markers contained in the urine and to measure these chemically modified polypeptide markers. The polypeptide markers according to the present invention have a molecular mass between 400 and 20000 Da, particularly between 700 and 14000 Da, more particularly between 800 and 11000 Da.

Preferred polypeptide markers according to the present invention are listed in tables 1 to 22, particularly in tables 1 to 21, more particularly in tables 1 to 13.

Preferred polypeptides for use as internal standards are listed in tables 23 to 24.

Preferred are also polypeptide markers which are listed in table 1, but not in table 14 and/or 15 and/or 16 and/or 17 and/or 18 and/or 19 and/or 20 and/or 21 and/or 22.

Preferred are also polypeptide markers which are listed in table 2, but not in table 14 and/or 15 and/or 16 and/or 18.

Preferred are also polypeptide markers which are listed in table 3, but not in table 14 and/or 16 and/or 18.

Preferred are also polypeptide markers which are listed in table 4, but not in table 14 and/or 16 and/or 19.

Preferred are also polypeptide markers which are listed in table 5, but not in table 14 and/or 16 and/or 18 and/or 19.

Preferred are also polypeptide markers which are listed in table 6, but not in table 14 and/or 16.

Preferred are also polypeptide markers which are listed in table 7, but not in table 14 and/or 16 and/or 17.

Preferred are also polypeptide markers which are listed in table 8, but not in table 14 and/or 16.

Preferred are also polypeptide markers which are listed in table 9, but not in table 14 and/or 16 and/or 20 and/or 21.

Preferred are also polypeptide markers which are listed in table 10, but not in table 14 and/or 16.

Preferred are also polypeptide markers which are listed in table 11, but not in table 14 and/or 16.

Renal disease according to the present invention relates to any kind of renal disease or kidney dysfunction known to the person skilled in the art, for example IgA-nephropathy, MGN (membranous glomerulonephritis), MCD (minimal-change disease), FSGS (focal-segmental glomerulosclerosis), or diabetic nephropathy. Particularly, renal disease relates to a glomerulopathy such as IgA-nepluopathy, MGN, MCD, or FSGS. Even more particularly renal disease relates to IgA-nephropathy, MCD, or FSGS. Most particularly, renal disease relates to IgA-nephropathy

The glomerulopathies are a subgroup of renal diseases. Glomerulopathies comprise a several diseases of different etiology. Glomerolopathies are characterized by pathomorphological changes in malpighian corpuscles, glomerulus, and Bovvman's capsule. As a consequence of these changes, further pathomorphological changes may appear in other parts of the nephron and interstice.

IgA-nephropathy is also known as Berger-Nephritis. IgA-nephropathy is the most common glomerulopathy. It may be a specific, kidney-limited, form of purpura Schoenlein-Henoch (also known as anaphylactoid purpura) with increased plasma concentration of IgA. The histopathology includes all forms of glomerular lesions and deposits of IgA in the mesangium. Clinically, IgA nephropathy presents as micro- and macro-hematouria. Therapy may be attempted with ACE inhibitors and omega-3 fatty acids. Progression of the disease occurs over the course of several years and includes transition into progressive renal insufficiency.

MGN is characterized by thickening of the basal membrane and granular subepithelial IgG deposits. MGN becomes frequently manifest in the between the age of 40 and 50. It is frequently caused by medicaments, e.g. gold, D-penicillamine, or ACE inhibitors. Therapy of MGN may be attempted with glucocorticoids or cyclophosphamide. MGN is a nephrotic syndrome, a transition into progressive renal insufficiency may take several years.

MCD is also known as lipoid nephrosis. MCD is the most common cause of a nephrotic syndrome in children. The etiology of the disease is unknown. Histologically, no or only very discrete changes can be found. Therapy of MCD may include treatment with glucocorticoids, cyclosporin A, or cyclophosphamide. In children, the disease spontaneously heals in 90% of the cases, in adults in 50% of the cases. A transition into FSGS is possible.

FSGS is also known as IgM-nephropathy. FSGS is typically characterized by deposits of IgM and C3 in the mesangium. Clinically, it becomes manifest as a nephrotic syndrome. Therapy of FSGS may include treatment with glucocorticoids, cyclosporin A, or cyclophosphamide. Prognosis is poor and includes transition into progressive renal insufficiency.

Diabetic nephropathy is also known as diabetic glomerulosclerosis. Diabetic nephropathy is the most common cause for requirement of dialysis treatment.

In summary, it is evident that renal diseases include a variety of diseases which may show quite similar histology. However, etiology, treatment, and prognosis can be quite different for each disease. For example, IgA-nephropathy requires different treatment from any other glomerulopathy described above: In IgA-nephropathy, treatment with ACE inhibitors may be attempted, which would not be recommendable in the case of MGN. Therefore, fast and reliable diagnosis is of great importance for treatment.

In the context of the present invention, diagnosing or diagnosis means that, for an individual patient, the probability of having the respective disease is determined.

Diagnosis may also include confirming a preliminary diagnosis, particularly a preliminary diagnosis established by a different method.

Furthermore, in a preferred embodiment, diagnosis according to the present invention particularly relates to “differential diagnosis”. The term “differential diagnosis” relates to distinguishing between two different diseases, i.e. to determining for an individual patient the probability of having a certain first disease as compared to having a certain second disease. More particularly, differential diagnosis according to the present invention relates to distinguishing between at least two renal diseases chosen from the group consisting of IgA-nephropathy, MGN, MCD, FSGS, and diabetic nephropathy.

In another embodiment, the present invention relates to a method for the differential diagnosis of a renal disease, the method comprising:
a) measuring the presence or the absence of a polypeptide marker in a urine sample, wherein the polypeptide marker is selected from the group of polypeptide markers shown in table 1 to 22, and
b) comparing the probability of the presence of this marker in a disease patient to the probability of the presence of this marker in a control patient, wherein
c1) if the probability of the presence of this marker in a disease patient is higher than the probability of the presence of this marker in a control patient, the presence of this marker is indicative for a higher probability of having the disease rather than the control condition, or
c2) if the probability of the presence of this marker in a disease patient is lower than the probability of the presence of this marker in a control patient, the absence of the marker is indicative for a higher probability of having the disease rather than the control condition.

Preferably, the individual probabilities according to step b) are as indicated in the tables.

The term “measuring” according to the present invention relates to determining the presence of a polypeptide or other substance of interest.

The decision whether a polypeptide marker is present or absent may depend on definition of a suitable threshold value. The threshold value can either be defined through the sensitivity of the method of measurement, or it can be defined at will. The threshold in the context of the present invention is 25 fmol/μl in a sample which has been injected into a mass spectrometer according to Example 1. However, this threshold may be the same when other methods are used. This threshold coincides with the detection threshold of a typical mass spectrometer. This threshold corresponds approximately to a concentration of the polypeptide marker in the urine sample of 50-5000 pmol/l. If different thresholds are to be used (e.g. when using another detection method), the corresponding probabilities may differ, but can easily be established by the person skilled in the art.

The “disease patient” according to the present invention is suffering from a renal disease. Particularly, the disease is at least one from the group consisting of IgA-nephropathy, MGN, MCD, FSGS, and diabetic nephropathy.

The “control patient” can either be healthy or suffering from a disease different from the one the disease patient is suffering from, i.e. the control patient can either represent the healthy condition or a disease or group of diseases. Particularly, the represented disease is at least one from the group consisting of IgA-nephropathy, MGN, MCD, FSGS, and diabetic nepmopathy.

Tables 1 to 14, 16, 20, 21, and 22 list the probability (also designated as “frequency”) of a given polypeptide marker being present in a urine sample of a healthy control patient or a control patient suffering from a certain disease. The discrimination factor indicates the difference between the probability of presence in the disease as compared to a given control condition. The discrimination factor can easily be calculated from the respective probabilities. The higher the discrimination factor, the better is the potential of the given marker to distinguish between the disease and the control condition. An absolute value of the discrimination factor of 0.40 or higher is preferred.

The person skilled in the art is able to establish similar tables for the polypeptide markers by himself and/or to refine the data contained in the tables, e.g. based on further patient data and/or according to different thresholds for the presence of the polypeptide marker.

For diagnosis, the probability of the presence of the polypeptide marker in a disease patient is compared to the probability of the presence of this marker in a control patient, wherein the individual probabilities are as indicated in the tables. If the probability of the presence of this marker in a disease patient is higher than the probability of the presence of this marker in a control patient, then the presence of this marker in the sample is indicative that the patient from whom the sample originates has a higher probability of having the disease rather than the control condition. If the probability of the presence of this marker in a disease patient is lower than the probability of the presence of this marker in a control patient, then the absence of this marker in the sample is indicative that the patient from whom the sample originates has a higher probability of having the disease rather than the control condition.

For example, a given marker may have a probability of 73% of being present in a control representing IgA-nephropathy but a probability of 0% of being present in a control representing the healthy condition. If this marker is present in the sample, then the individual is diagnosed as having a 73% probability of suffering from IgA-nephropathy as compared to being healthy. If this marker is not present in the sample, then the individual is diagnosed as having a 73% probability of being healthy instead of suffering from IgA-nephropathy.

Thus, diagnosis can be established according to statistical methods familiar to the person skilled in the art.

The invention can be carried out using only one of the polypeptide markers or using a plurality of the polypeptide markers. Preferably, presence of a plurality of polypeptide markers is measured. Preferably at least 3 of the markers, more preferably at least 10 of the markers, even more preferably at least 20, most preferred at least 50 of the markers according to the present invention are measured.

An advantage of the present invention is that it provides a multitude of suitable markers. Measuring a plurality of markers can increase both sensitivity and selectivity of diagnosis. Therefore, also markers which show low discrimination factors between the disease and control can be used for diagnosis if they are combined with other markers.

If a plurality of polypeptide markers is used, a “pattern” is be generated which contains the information about the presence for each marker measured. This pattern can then be compared to the pattern of probabilities of presence of the polypeptide markers in a disease or control patient. Each table represents a pattern of probabilities of finding given polypeptide markers in certain disease and control patients.

Therefore, in a preferred embodiment, the present invention relates to a method for the differential diagnosis of a renal disease, the method comprising:
a) establishing a pattern of presence or absence for a plurality of polypeptide markers in a urine sample, wherein at least one polypeptide marker is selected from the group of polypeptide markers shown in table 1 to 22, and
b) comparing the probability of finding this pattern in a disease patient to the probability of finding this pattern in a control patient, wherein
c1) if the probability of finding the pattern in a disease patient is higher than the probability of the finding the pattern in a control patient, finding this pattern is indicative for a higher probability of having the disease rather than the control condition, or
c2) if the probability of finding the pattern in a disease patient is lower than the probability of the finding the pattern in a control patient, finding this pattern is indicative for a lower probability of having the disease rather than the control condition, or

Preferably, the individual probability for the at least one polypeptide marker according to step b) is as indicated in the tables.

Comparison of the found pattern with the probability of finding the pattern in a disease or control patient can be performed according to statistical methods known in the art. Preferably, automated methods are employed, e.g. CART-analysis, random forest analysis, and support vector machines (SVM, see e.g, Xiong. M., et al. (2001). Biomarker identification by feature wrappers. Genome Research vol. 11, p. 1878-1887). Comparison can also be performed simultaneously for several different patterns and the probability of finding them.

Thus, the measured pattern is typically compared to the probability of finding the pattern in at least two different conditions. An example for diagnosis and differential diagnosis of renal diseases according to this method is shown in FIG. 3.

If necessary, the urine samples may be pre-treated before measurement of the polypeptide marker. Particularly, lipids, nucleic acids or polypeptides may be purified from the sample according to methods known in the art, including filtration, centrifugation, or extraction methods such as chloroform/phenol extraction.

Measuring the presence of a polypeptide marker can be done by any method known in the art.

Preferred methods include gas phase ion spectrometry, such as laser desorption/ionization mass spectrometry, surface enhanced laser desorption/ionization time-of flight mass spectrometry (SELDI-TOF MS) and CE-MS. These spectrometry methods allow to measure the polypeptide markers without the need for ligands such as antibodies or aptamers.

Urine sample generally are highly complex, i.e. they contain numerous polypeptides. In case of high complexity, a spectrometric analysis becomes difficult. To reduce the complexity of the sample, the polypeptides contained in the sample may be separated by any suitable means, e.g. by electrophoretic separation, affinity-based separation, or separation based on ion exchange chromatography. Particular examples include gel electrophoresis, two-dimensional polyacrylamide gel electrophoresis (2D-PAGE), capillary electrophoresis, metal-affinity chromatography, immobilized metal-affinity chromatography (IMAC), affinity chromatography based on lectins, liquid chromatography, high pressure liquid chromatography (HPLC), and reversed-phase HPLC, cation exchange chromatography, and selectively binding surfaces (such as the surfaces used in SELDI-TOF, see below).

2D-PAGE is commonly used for polypeptide separation and can be combined with mass spectrometry (MS) yielding identification of individual polypeptides. Over 1000 protein spots can be discerned with 2D-PAGE. However, each single spot must be analyzed separately by MS/MS for identification.

SELDI (surface enhanced laser desorption/ionization) time-of-flight mass spectrometry is currently applied in many fields of biomedical sciences.

In the SELDI system, the ProteinChip Arrays are the most important component. They are narrow metal strips carrying 8 or 16 spots in a row on the surface. Samples to be analyzed are directly applied to the spots, either as a standing drop or in volumes up to 500 μl, by using sample holders called “bioprocessors” as supporting units. They are placed onto the arrays during incubation and washing steps and removed again afterwards. The different types of arrays belong to two main series: chromatographic arrays, presenting hydrophobic, hydrophilic, cation-exchanging, anion-exchanging or immobilized metal ion affinity-surfaces, and preactivated arrays with chemical groups to allow the covalent coupling of proteins. Preferably, a chip with cation-exchange surfaces is used. As the ProteinChip Arrays do not only support the sample but specifically interact with the biomolecules, the composition of the analyte depends on the array type used and the washing conditions applied. This explains why the SELDI-process can be defined as a further development of the traditional MALDI (matrix assisted laser desorption/ionization)-technique. In the SELDI-process, only on those polypeptides are measured that actually bind to the chip surface.

After binding of sample proteins, the energy absorbing matrix is applied to each spot. The matrix rapidly crystallizes and the analysis can start immediately.

The ProteinChip Arrays are placed into the ProteinChip Reader for analysis. The reader is a TOF (time-of-flight) mass spectrometer in which the proteins are desorbed and ionized with the help of a laser beam. As the crystallized proteins are equally distributed on the spot surface, the ionizing laser beam always hits a representative average of the molecules in the analyte, allowing quantitative calculations. After ionization, the proteins are accelerated by an electric field to fly down the flight tube, before reaching the detector. The flight time between the laser striking the array surface and the molecules reaching the detector at the end of the flight tube enables the system to accurately determine the mass of the protein species present in the sample (for more detailed information on the method see the following review; Merchant M and Weinberger S R (2000). Recent advancements in surface-enhanced laser desorption/ionization—time of flight mass spectrometry. Electrophoresis vol. 212, p. 1164-1177).

However, the most preferred method is CE-MS, in which capillary electrophoresis (CE) is coupled to mass spectrometry (MS). CE-MS has been described in detail elsewhere (see e.g. German patent application DE 100 21 737, and Kaiser, T., et. al., Capillary Electrophoresis coupled mass spectrometry to establish polypeptide patterns in dialysis fluids. J Chromatogr A, vol. 1013, p. 157-171(2003)).

CE is known to the person skilled in the art. In brief, the sample is loaded onto an electrophoresis capillary and a voltage of up to 50 kV, typically up to 30 kV, is applied. Typical capillaries are fused silica capillaries, i.e. glass capillaries comprising an outer sheath as mechanical support and to improve mechanical flexibility, e.g. a sheath made of thermoplastic material. Typically, the capillary is untreated, i.e. it shows hydroxy-groups on its inside. However, the capillary may also be coated on the inside. E.g., hydrophobic coating can be used to improve discriminatory power. In addition to the voltage, also pressure may be applied, which is typically in the range of 0 to 1 psi. The pressure can also be applied or increased during the run.

To improve discriminatory power, also a stacking protocol can be applied when loading the sample: Before loading of the sample, a base is loaded, then the sample is loaded, then an acid. The principle is to capture the analyte ions between a base and an acid. If voltage is applied, the positively charges analyte ions move towards the base. There, they get negatively charged and move into the opposite direction towards the acid, where they get positively charged. This stacking repeats itself until acid and base are neutralized. Then, the separation starts from a well concentrated sample.

The sample is contained in an appropriate buffer in which polypeptides are soluble, e.g. phosphate buffer. For CE-MS coupling, it is preferred to use volatile solvents and to work under mostly salt-free conditions to avoid contamination of the MS. Examples comprise acetonitrile, isopropanol, methanol, and the like. The solvents can also be combined with water and a weak acid (e.g. 0.1% formic acid), the latter to protonate the analyte. The polypeptides in the sample are separated according to size and charge, which determine the run-time in the capillary. CE is characterized by high separating power and short time of analysis.

For subsequent MS analysis, either fractions collected from the CE can be analyzed as separate batches or, preferably, the CE system can be coupled via a suitable interface to the mass spectrometer to allow continuous flow analysis. Alternatively, the flow from the CE may be used to generate continuous “separation tracks”, which can be analyzed separately.

In the mass spectrometer, ions generated from the sample are analyzed according to the mass/charge (m/z) quotient. Using mass spectrometry, it is possible to routinely analyze 10 fmol (i.e. 0.1. ng of a 10 kDa polypeptide) with a precision of ±0.01%. Experimentally, is possible to analyze even less than 0.1 fmol.

Any type of mass spectrometer can be used. In mass spectrometers, an ion-generating device is coupled with an suitable analyzer. For example, the electrospray ionization (ESI) interfaces are most commonly used to produce ions from liquid samples, whereas MALDI is most commonly used to produce ions from individually processed samples. Different kinds of analyzers are available, e.g. ion trap analyzers or time-of-flight (TOF) analyzers. Both ESI and MALDI can be combined with essentially all types of mass spectrometers, although ESI has usually been combined with ion traps, whereas MALDI has usually been combined with TOF.

A preferred CE-MS method according to the present invention includes capillary electrophoresis coupled online via ESI to a TOF analyzer.

The CE-MS technique permits to measure the presence of several hundred polypeptide markers simultaneously in a short time in a small volume with high sensitivity. Once the presence of the polypeptide markers has been measured, a pattern of the measured polypeptide markers is generated and can be compared to a disease pattern by any of the methods described further above. However, in many cases it will be sufficient for diagnosis to measure only one or a limited number of the markers.

The polypeptide sequences can be determined according to methods well-known to the person skilled in the art (see e.g. C. S. Spahr et al. (2001). Towards defining the urinary proteome using liquid chromatography-tandem mass spectrometry. I. Profiling an unfractionated tryptic digest, Proteomics vol. 1, p. 93-107).

Depending on the type of polypeptide marker, it is possible to measure its presence or absence by further means. For example, if the polypeptide is biologically active, its presence may be determined by cellular or enzymatic assays.

Presence of a polypeptide can also be determined by use of ligands binding to the polypeptide of interest. Binding according to the present invention includes both covalent and non-covalent binding.

A ligand according to the present invention can be any peptide, polypeptide, nucleic acid, or other substance binding to the polypeptide of interest. It is well known that polypeptides, if obtained or purified from the human or animal body, can be modified, e.g. by glycosylation. A suitable ligand according to the present invention may bind the polypeptide also via such sites.

Preferred ligands include antibodies, nucleic acids, peptides or polypeptides, and aptamers, e.g. nucleic acid or peptide aptamers. For many polypeptides, suitable ligands are commercially available. Furthermore, methods to generate suitable ligands are well-known in the art. For example, identification and production of suitable antibodies or aptamers is also offered by commercial suppliers.

The term “antibody” as used herein includes both polyclonal and monoclonal antibodies, as well as fragments thereof, such as Fv, Fab and F(ab)₂ fragments that are capable of binding antigen or hapten.

Preferably, the ligand should bind specifically to the polypeptide to be measured. “Specific binding” according to the present invention means that the ligand should not bind substantially to (“cross-react” with) another polypeptide or substance present in the sample investigated. Preferably, the specifically bound protein or isoform should be bound with at least 3 times higher, more preferably at least 10 times higher and even more preferably at least 50 times higher affinity than any other relevant polypeptide.

Non-specific binding may be tolerable, particularly if the investigated peptide or polypeptide can still be distinguished and measured unequivocally, e.g. according to its size on a Western Blot, or by its relatively higher abundance in the sample.

A method for measuring the presence of a polypeptide of interest may comprise the steps of (a) contacting a polypeptide with a specifically binding ligand, (b) (optionally) removing non-bound ligand, (c) measuring the presence or amount of bound ligand.

Binding of the ligand can be measured by any method known in the art. First, binding of a ligand may be measured directly, e.g. by NMR or surface plasmon resonance. Second, the ligand also serves as a substrate of an enzymatic activity of the peptide or polypeptide of interest, an enzymatic reaction product may be measured (e.g. the presence of a protease can be measured by measuring the amount of cleaved substrate, e.g. by Western Blot). Third, the ligand may be coupled covalently or non-covalently to a label allowing detection and measurement of the ligand.

Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.).

The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus.

Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels.

Enzymatically active labels include e.g. horseradish peroxidase, alkaline phosphatase, beta-Galactosidase, Luciferase, and derivatives thereof. Suitable substrates for detection include di-amino-benzidine (DAB), 3,3″-5,5″-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art.

Typical fluorescent labels include fluorescent proteins (such as GFP and its derivatives), Cy3, Cy5, Texas Red, Fluorescein, the Alexa dyes (e.g. Alexa 568), and quantum dots.

Typical radioactive labels include ³⁵S, ¹²⁵I, ³²P, ³³P, and the like.

Thus, suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting), can be used alone or in combination with labeling or other detection methods as described above.

The ligand may also be present on an array. Said array contains at least one additional ligand, which may be directed against a peptide, polypeptide or a nucleic acid of interest. Said additional ligand may also be directed against a peptide, polypeptide or a nucleic acid of no particular interest in the context of the present invention. Preferably, ligands for at least five, more preferably at least 10, even more preferably at least 20 polypeptide markers according to the present invention are contained on the array.

According to the present invention, the term “array” refers to a solid-phase or gel-like carrier upon which at least two compounds are attached or bound in one-, two- or three-dimensional arrangement. Such arrays (including “gene chips”, “protein chips”, antibody arrays and the like) are generally known to the person skilled in the art and typically generated on glass microscope slides, specially coated glass slides such as polycation-, nitrocellulose- or biotin-coated slides, cover slips, and membranes such as, for example, membranes based on nitrocellulose or nylon.

The array may include a bound ligand or at least two cells expressing each at least one ligand.

It is also contemplated to use “suspension arrays” as arrays according to the present invention (Nolan I P, Sklar L A. (2002). Suspension array technology: evolution of the flat-array paradigm. Trends Biotechnol. vol. 20(1), p. 9-12). In such suspension arrays, the carrier, e.g. a microbead or microsphere, is present in suspension. The array consists of different microbeads or microspheres, possibly labeled, carrying different ligands.

The invention further relates to a method of producing arrays as defined above, wherein at least one ligand is bound to the carrier material in addition to other ligands.

Methods of producing such arrays, for example based on solid-phase chemistry and photolabile protective groups, are generally known (U.S. Pat. No. 5,744,305). Such arrays can also be brought into contact with substances or substance libraries and tested for interaction, for example for binding or change of conformation. Therefore, arrays comprising a polypeptide marker according to the present invention may be used for identifying ligands binding specifically to said peptides or polypeptides.

To determine the sequence of a polypeptide, it should be purified to the highest level achievable. However, the polypeptide does not need to be completely isolated. For example, it is enough to have the polypeptide detectable as a coomassie-stained band in a polyacrylamide gel. The corresponding gel piece can then be cut out and used for the next identification steps. After purification of the polypeptide, it can be enzymatically digested with trypsin and the molecular weights of the resulting fragments determined using any suitable method, for example mass spectrometry. Using mass spectrometry, each polypeptide displays a characteristic “fingerprint” of fragments allowing its identification by database searches. In case that the polypeptide to be identified is not present in the database or if the researcher wants to have a closer characterization for any reasons, the polypeptide fragments can also be sequenced according to methods known in the art.

CE-MS allows particularly easy determination of the polypeptide sequences. The capillary electrophoresis elution time for each marker is listed in the tables. Thus, it is possible to collect the fraction containing the polypeptide at relatively high purity. If a single fraction contains insufficient material, fractions of more than one experiment may be pooled.

Sequences of some of the polypeptide markers are listed as SEQ ID NO: 1 to 5. Their masses as measured by CE-MS and their respective sequences are as follows:

| SEQ | mass | | |
|---|---|---|---|
| ID: | [Da] | sequence | Description |
| 1 | 8765,9 | FTFHADICTLSEKERQIKKQTALVEL | fragment of human |
| | | VKHKPKATKEQLKAVMDDFAAFVEKC | albumin, C-terminus |
| | | CKADDKETCFAEEGKKLVAASQAALG | amino acids 531-609. |
| | | L | |
| 2 | 10046,3 | TYVPKEFNAETFTFHADICTLSEKER | fragment of human |
| | | QIKKQTALVELVKHKPKATKEQLKAV | albumin, C-terminus, |
| | | MDDFAAFVEKCCKADDKETCFAEEGK | amino acids 520 to |
| | | KLVAASQAALGL | 609. |
| 3 | 950.0 | GGRPSRPPQ | fragment of Salivary |
| | | | proline-rich protein |
| 4 | 1292.5 | GFRHRHPDEAA | fragment of alpha |
| | | | fibrinogen |
| 5 | 1448.8 | GLITLIGINPSLHT | fragment of olfactory |
| | | | receptor 8B4 |

### FIGURE LEGENDS

FIG. 1 Depiction of the information from a crude CE-MS analysis (A) as a three dimensional contour plot (left side). Here a contour plot of urine from a healthy volunteer is shown, mass per charge on the Y-axis against the retention time in min (X-axis), signal intensity color coded. Next, the signal to noise is calculated and the noise removed, thus leaving only actual signals (B). The software calculates the actual mass (C) based on both isotopic distribution and conjugated masses. This results in a table of up to 1500 polypeptides defined via their mass and retention time. As an example, bottom right shows 17 polypeptides found in the sample. CE-t, CE-time (migration time); int., intensity; m.p.c., mass per charge, cal. m., calculated mass.

FIG. 2 Contour plots of polypeptides (actual masses) for healthy subjects (NC) and for patients with focal-segmental glomerulosclerosis (FSGS), minimal-change disease (MCD) and membranous glomerulonephritis (MGN) are shown. The upper mass limit for each plot (i.e. the maximum value along the X-axis) is indicated on the top left of each plot. As evident, the contour plots differ significantly between the healthy subjects and the renal disease groups.

FIG. 3 Flow sheet for diagnosis and differential diagnosis of renal diseases (example). Samp., sample; MS-dat., MS-data; Disea., disease; Y, yes; N, no; n.d., no disease; d.n., diabetic nephropathy, FSGS, FSGS; MGN, MGN; MCD, MCD; IgA, IgA-nephropathy, diff., different diagnosis.

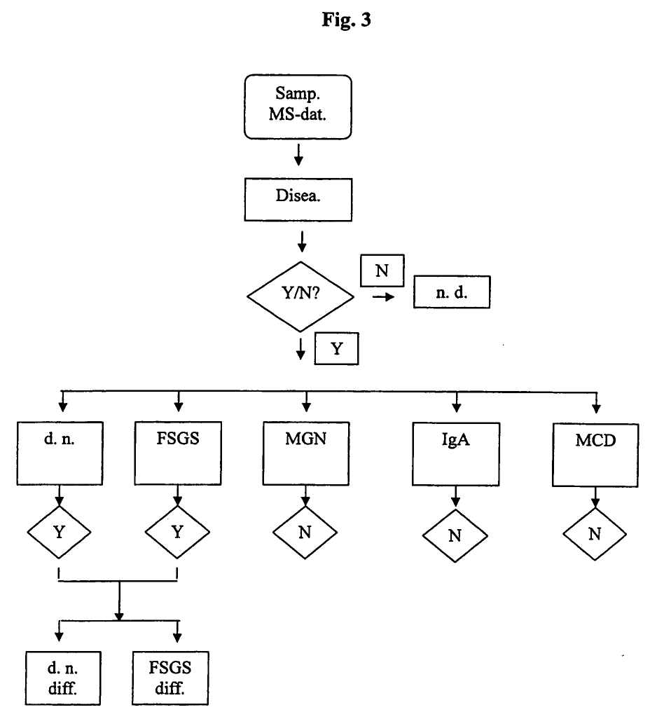

The invention is further illustrated by the following examples:

### EXAMPLE 1

Participants:

After local Ethics Committee approval, informed consent was obtained from all participants. We examined a group of 57 healthy individuals with normal renal function in order to establish normal urinary protein patterns with CE-MS. In addition, we studied 44 patients with biopsy-proven minimal-change disease (n=16; MCD), membranous glomerulonephritis (n=18; MGN), and focal-segmental glomerulosclerosis (n=10; FSGS) (Table 1).

CE-MS Analysis:

Spot urine samples were collected from all participants in the morning after voiding the first urine. Samples were prepared as described in detail elsewhere (Wittke S, Fliser D, Haubitz M, et al: Determination of peptides and proteins in human urine with CE-MS—suitable tool for the establislunent of new diagnostic markers. J Chromatogr A 1013:173-181, 2003). The CE-MS analysis was established as described previously (Kaiser T, Hermann A, Kielstein J T, et al: Capillary Electrophoresis coupled mass spectrometry to establish polypeptide patterns in dialysis fluids. J Chromatogr A 1013: 157-171, 2003), using a Beckman Coulter PAC/E system coupled to a Mariner TOF mass spectrometer (ABI). CE capillaries were from Beckman, ID/OD 75/360 μm and 90 cm in length. The mobile phase used contained 30% methanol and 0.5% formic acid in water. The same liquid was used for the sheath flow, which was applied at 2 μl/min. Sample injection was performed with pressure: 1 psi for 20 sec. Under these conditions about 100 nl of sample could be injected. For sample stacking, the following protocol was applied: injection of 1M NH₃ for 7 sec., injection of sample, injection of 2M formic acid for 5 sec. The subsequent CE-MS run was performed at +30 kV with the sequence of the following pressures: 40 min at 0 psi, 2 min at 0.1 psi, 2 min at 0.2 psi, 2 min at 0.3 psi, 2 min at 0.4 psi, 80 min at 0.5 psi. For diagnosis of IgA-nephropathy, the following pressure sequence was used: 40 min at 0.3 psi, 2 min at 0.4 psi, 2 min at 0.6 psi, 2 min at 0.8 psi, 80 min at 1 psi. After each run, the CE capillary was rinsed for 5 min with 0.1 M NaOH, followed by 5 min with water and 5 min with running buffer.

Statistical Analysis:

For discrimination between healthy subjects and different groups of patients with renal diseases we used the method of Random Forests and the corresponding S-Plus program version 6/2002 Breiman L: Random Forests. (http://oz.berkeley.edu/users/breiman/randomforest2001.pdf). In this procedure, a series of PP subsets of fixed size is selected randomly from all candidate PP. For each subset, a classification tree as described in the Classification and Regression Tree (CART) analysis is generated (Steinberg D, Colla P; CART—Classification and Regression trees. San Diego, Calif., Salford Systems 1997), resulting in a classification rule. The forest prediction is the unweight plurality of class votes of the series of classification rules. Over-fitting is not generated due to large numbers of subset selections. The estimated generalisation error is unbiased due to the method of “out of bag” (oob) estimation: each tree is grown on a bootstrap sample of cases of the learning sample and the validation is estimated on the basis of those cases not selected in the bootstrap sample.

Further, discrimination between groups was also performed using support vector machines. This tool has the advantage of discriminating data in high dimensional parameter space. Its fast and stable algorithms showed good performance in the evaluation of clinical markers (Dieterle F, Muller-Hagedorn S, Liebich H M, Gauglitz G; Urinary nucleosides as potential tumor markers evaluated by learning vector quantization. Artif Intell Med 28:265-279, 2003) and different areas of biological analyses like DNA arrays (Brown M P, Grundy W N, Lin D, et al: Knowledge-based analysis of microarray gene expression data by using support vector machines. Proc Natl Acad Sci USA 97:262-267, 2000).

Normal Urinary Polypeptide Pattern Analysed with CE-MS:

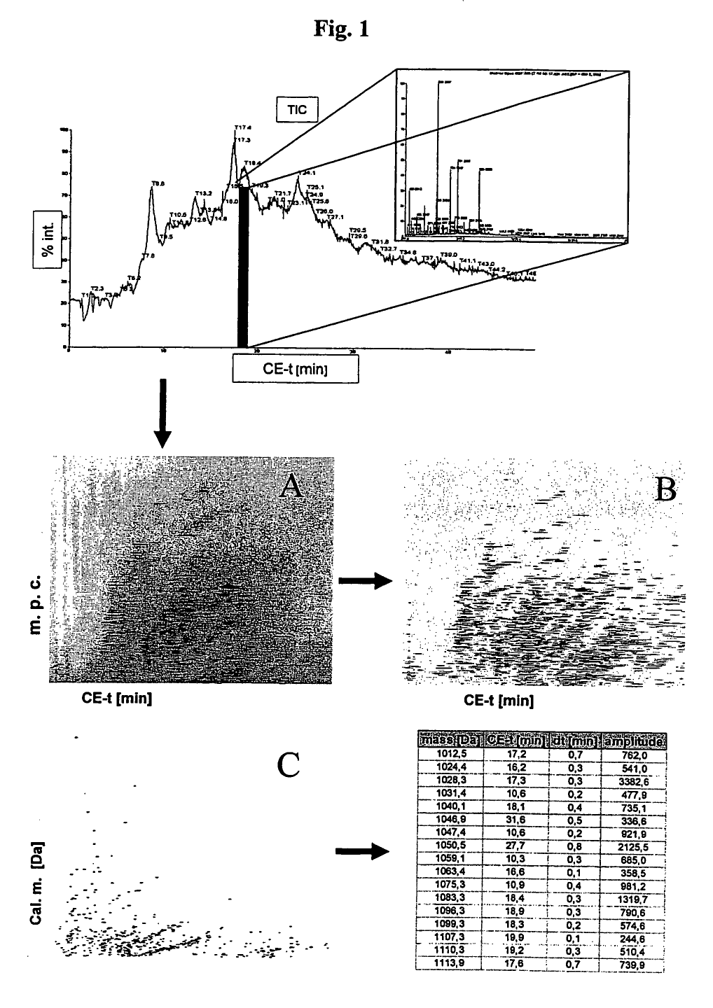

A graphical depiction (contour plot) of a typical sample is presented in FIG. 1 (raw data). In one individual sample, between 900 and 2500 PP with molecular weights from 800 up to 30.000 Dalton were detected. Under the conditions used for CE polypeptides with higher molecular weights tend to precipitate. Thus larger proteins in general cannot be detected, although some (e.g. albumin) can be visualized. A list of polypeptides present with high probability that were chosen as internal standards to assure sample comparability is shown in table 23. For analysis of protein-rich samples, such as samples from suspected IgA-nephropathy patients, higher pressure was applied and the polypeptides according to table 24 were preferred as internal standards. Repeated analyses of identical samples did not reveal any significant differences under identical conditions of the CE-MS run for an individual sample.

The subsequent electronic data manipulation for one example is summarized in FIG. 1. Each run results in the crude spectrum depicted in the upper part of FIG. 1 and is composed of single spectra (blow up FIG. 1) generated every 3 seconds. CE-MS peaks were identified in the first data analysis run (FIG. 1A). Next, the charge of each peak was ascertained utilizing both isotopic distribution and conjugated peaks (FIG. 1B). As a result, conjugated peaks were summarized in one single peak and the real mass was calculated, as shown in FIG. 1C. Initially, the samples were spiked with external standards of known mass. This allowed subsequent definition of internal standards of PP present with high probability in the urine samples. Thus the CE-time could be normalized to the internal standards. By applying this technique on an average urine sample, roughly 1000 PP can be detected and described/identified by the two parameters mass and CE-migration time.

The examination of urine obtained from healthy subjects led to the establishment of peaks defined by actual mass and CE-time of the PP detected, so-called peak lists, and contour plots for each individual. The individual peak lists were deposited in an MS-Access database and the probability of each of the PP to appear in a single sample was calculated. One-hundred seventy-three PP were present in over 90% of the control samples examined. In addition, 156 PP were present in more than 75% of the samples, while additional 361 PP were found in over 50% of samples from the healthy individuals. These 690 PP were found in more than 50% of all samples obtained from healthy subjects and were used to establish a “normal PP pattern”.

Urine from Patients with Renal Diseases Analyzed with CE-MS:

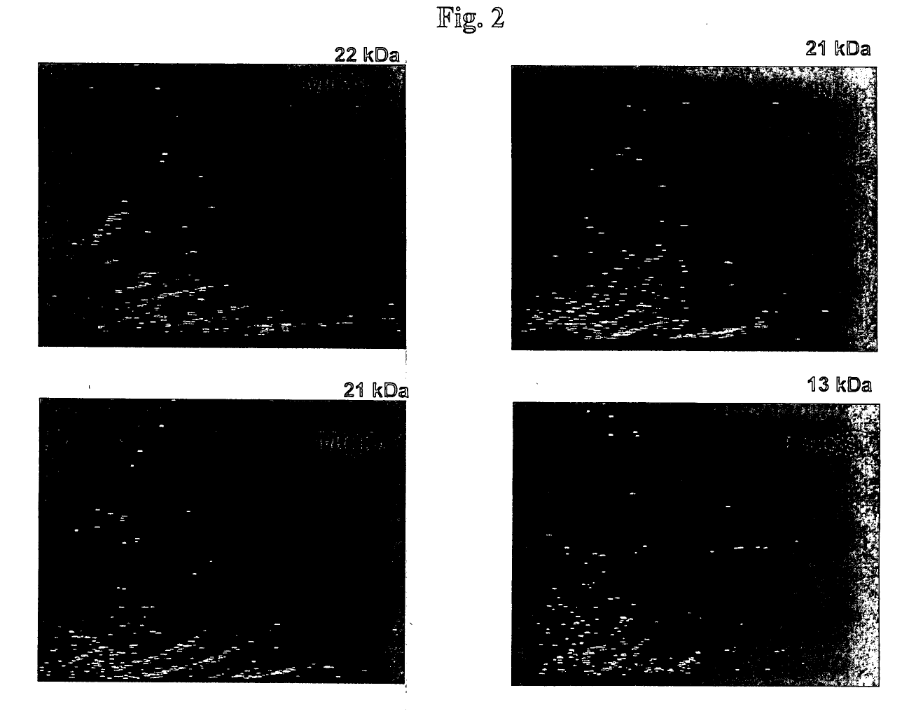

Data from the individual runs of 44 patients were sub-grouped in the three disease groups and analyzed. The values from these databases, representing typical PP patterns, were subsequently compared. Significant homology of the protein patterns present in urine samples from each patient group was found within the groups. Typical examples of urinary PP patterns from patients with MCD, FSGS, and MGN are shown in FIG. 2. Each disease presents a typical protein contour plot, revealing more than 500 PP. Subsequently, the data from the three groups were compared with those obtained in healthy subjects. Table 16 shows 124 PP found in the urine of more than 95% of healthy subjects and reveals the differences to patients with MCD, FSGS, and MGN.

Statistical analysis for discrimination of healthy individuals and patients with renal disease using CE-MS data was applied. A list of 800 PP, present with more than 50% probability in either disease group was chosen for Random Forest analysis. The correct classification rate for the discrimination between healthy subjects and renal patients was 96.5%, as shown in the following list:

| | Healthy subjects | Renal patients | Classification error |
|---|---|---|---|
| Class | (n = 57) | (n = 44) | [%] |
| classification as | 56 | 2 | 3.5 |
| healthy | | | |
| classification as | 1 | 42 | 2.3 |
| patients | | | |

After cross-validation a sensitivity of 81.3% and a specificity of 94.3% could be obtained. Discrimination of the disease groups was achieved in the learning sample. However, most likely due to the small number of FSGS patients, these could not be discriminated from MCD when applying cross-validation. Hence, FSGS and MCD were combined into one group. For the discrimination between healthy subjects, MCD/FSGS and MGN, four PP were selected by CART from the list to build a classification tree with five terminal nodes (table 15). The correct classification rate in the learning sample is 94.1%. After cross-validation it reduces to 84.3% (93.8% for healthy controls, 71.4% for MCD/FSGS and 92.9% for MGN).

Alternatively, statistical analysis was performed using support vector machines on the same data; table 16 shows PP that were employed in this analysis. Using these PP, the correct classification was 98.0% after complete cross-validation. Table 17 depicts PP that were used to discriminate between MCD and MGN. Here the correct classification was 94.1% after complete cross-validation. Further, it was possible to separate patients with MCD and FSGS and patients with MGN and FSGS with (cross-validated) classification rates of 92.3% and 89.3%, respectively (tables 18 and 19). These results can be valued as a first approach using support vector machines to classify a limited number of patients. With increasing patients data the classification will further improve and become more stable. The results also indicate that for stable classification the number of applicable variables (polypeptides) depends on the number of cases (patients), hence an increase in patients will allow to use even more PP for classification.

TABLE 1

| molecular | migration | discrimination | frequency [%] | |
|---|---|---|---|---|
| weight [Da] | time [min] | factor | glomerulonephritis | healthy |
| 830.5 | 25.3 | 0.42 | 55 | 12 |
| 836.5 | 35 | 0.64 | 80 | 16 |
| 862.4 | 48.7 | 0.45 | 57 | 12 |
| 866.4 | 37.9 | 0.62 | 77 | 16 |
| 870.4 | 33.9 | 0.45 | 52 | 7 |
| 874.5 | 29.7 | 0.46 | 75 | 29 |
| 876.4 | 48.9 | −0.41 | 57 | 98 |
| 882.6 | 36.5 | −0.4 | 55 | 95 |
| 903.4 | 46.8 | −0.42 | 14 | 55 |
| 909.4 | 40.3 | 0.67 | 70 | 3 |
| 925.4 | 50.7 | 0.63 | 77 | 14 |
| 926.5 | 36.1 | −0.44 | 32 | 76 |
| 943.5 | 30 | 0.52 | 80 | 28 |
| 946.5 | 46.8 | −0.44 | 41 | 84 |
| 950.5 | 36 | 0.61 | 66 | 5 |
| 952.5 | 32 | 0.52 | 64 | 12 |
| 956.4 | 49.5 | −0.54 | 7 | 60 |
| 978.5 | 35 | 0.53 | 55 | 2 |
| 980.6 | 34.6 | −0.45 | 43 | 88 |
| 982.5 | 31.9 | 0.53 | 64 | 10 |
| 983.5 | 35.1 | 0.41 | 55 | 14 |
| 988.6 | 49.9 | −0.48 | 9 | 57 |
| 990.6 | 32 | 0.42 | 61 | 19 |
| 991.4 | 37.7 | 0.41 | 55 | 14 |
| 994.5 | 33.3 | 0.51 | 75 | 24 |
| 995.6 | 36.4 | 0.46 | 68 | 22 |
| 1000.5 | 34 | −0.58 | 36 | 95 |
| 1006.4 | 35.7 | −0.48 | 30 | 78 |
| 1008.5 | 34.4 | −0.5 | 43 | 93 |
| 1010.6 | 30.6 | −0.58 | 25 | 83 |
| 1013.4 | 39.3 | −0.43 | 9 | 52 |
| 1015.6 | 38.2 | 0.47 | 52 | 5 |
| 1028.6 | 37.8 | −0.55 | 43 | 98 |
| 1033.6 | 39.1 | 0.62 | 70 | 9 |
| 1038.6 | 34.4 | 0.43 | 57 | 14 |
| 1046.6 | 38.6 | −0.76 | 20 | 97 |
| 1047.6 | 30.4 | −0.44 | 41 | 84 |
| 1073.6 | 34.7 | 0.59 | 75 | 16 |
| 1075.6 | 29 | −0.43 | 20 | 64 |
| 1102.6 | 32.9 | 0.4 | 52 | 12 |
| 1108.6 | 29.8 | 0.47 | 86 | 40 |
| 1110.4 | 46.9 | −0.5 | 20 | 71 |
| 1121.6 | 42.3 | −0.46 | 16 | 62 |
| 1122.5 | 50.2 | −0.41 | 25 | 66 |
| 1135.6 | 42.7 | −0.61 | 5 | 66 |
| 1138.6 | 39.3 | 0.55 | 73 | 17 |
| 1139.6 | 32.2 | 0.52 | 59 | 7 |
| 1141.6 | 38 | −0.6 | 7 | 67 |
| 1157.6 | 28.5 | 0.5 | 80 | 29 |
| 1159.6 | 39 | −0.65 | 16 | 81 |
| 1163.7 | 38.1 | 0.47 | 50 | 3 |
| 1171.6 | 32.8 | 0.64 | 66 | 2 |
| 1182.6 | 47.2 | −0.45 | 20 | 66 |
| 1191.6 | 50.5 | −0.41 | 50 | 91 |
| 1191.8 | 18.3 | 0.56 | 66 | 10 |
| 1198.8 | 29.2 | 0.44 | 75 | 31 |
| 1203.7 | 24.7 | −0.52 | 5 | 57 |
| 1209.6 | 50.5 | −0.42 | 48 | 90 |
| 1211.6 | 31.3 | 0.5 | 66 | 16 |
| 1212.7 | 30.6 | 0.57 | 66 | 9 |
| 1219.6 | 37.3 | 0.57 | 77 | 21 |
| 1220.6 | 30.2 | 0.42 | 75 | 33 |
| 1223.5 | 51.6 | −0.6 | 30 | 90 |
| 1224.7 | 33.6 | −0.41 | 59 | 100 |
| 1225.7 | 41.3 | 0.45 | 50 | 5 |
| 1235.6 | 41.4 | −0.41 | 59 | 100 |
| 1237.7 | 41.6 | −0.49 | 18 | 67 |
| 1246.7 | 30.5 | −0.47 | 18 | 66 |
| 1256.6 | 53.4 | 0.41 | 55 | 14 |
| 1264.7 | 26.7 | 0.44 | 52 | 9 |
| 1268.6 | 53.7 | −0.47 | 5 | 52 |
| 1269.7 | 39.8 | 0.45 | 64 | 19 |
| 1270.5 | 52.5 | −0.51 | 5 | 55 |
| 1279.7 | 38.3 | 0.48 | 64 | 16 |
| 1280.6 | 51.9 | −0.53 | 9 | 62 |
| 1286 | 30.7 | 0.41 | 84 | 43 |
| 1292.5 | 53 | −0.59 | 27 | 86 |
| 1297.6 | 38.7 | 0.45 | 86 | 41 |
| 1302.7 | 31.8 | 0.54 | 86 | 33 |
| 1303.6 | 40.7 | −0.44 | 11 | 55 |
| 1311.8 | 31.5 | 0.58 | 77 | 19 |
| 1319.9 | 34.8 | 0.4 | 59 | 19 |
| 1324.2 | 40.5 | 0.5 | 59 | 9 |
| 1325.5 | 35.2 | 0.54 | 80 | 26 |
| 1333.8 | 38.8 | 0.65 | 82 | 17 |
| 1335.7 | 39.2 | 0.57 | 80 | 22 |
| 1338.7 | 29.6 | 0.46 | 57 | 10 |
| 1338.7 | 47.2 | 0.8 | 82 | 2 |
| 1350.7 | 50.3 | −0.48 | 2 | 50 |
| 1353.7 | 39.3 | −0.44 | 45 | 90 |
| 1354.8 | 45.6 | 0.55 | 93 | 38 |
| 1371.7 | 39.9 | 0.6 | 64 | 3 |
| 1371.8 | 19.3 | 0.63 | 89 | 26 |
| 1389.8 | 19.5 | 0.5 | 84 | 34 |
| 1390.7 | 41.1 | 0.45 | 64 | 19 |
| 1398.9 | 30.5 | 0.59 | 73 | 14 |
| 1401.8 | 46.2 | −0.53 | 9 | 62 |
| 1405.9 | 17.3 | 0.49 | 59 | 10 |
| 1408.9 | 26.8 | 0.42 | 52 | 10 |
| 1414.6 | 38.1 | 0.62 | 86 | 24 |
| 1415.7 | 33.3 | 0.45 | 50 | 5 |
| 1419.8 | 39.7 | 0.48 | 77 | 29 |
| 1424.9 | 35.4 | −0.46 | 25 | 71 |
| 1442.8 | 33.3 | 0.63 | 84 | 21 |
| 1444.6 | 37.8 | 0.53 | 82 | 29 |
| 1448.8 | 30.3 | 0.42 | 75 | 33 |
| 1465.9 | 28.8 | 0.59 | 66 | 7 |
| 1472.1 | 31.2 | 0.57 | 70 | 14 |
| 1474.9 | 16.9 | 0.65 | 77 | 12 |
| 1482 | 30.4 | 0.57 | 84 | 28 |
| 1484 | 30.4 | 0.58 | 89 | 31 |
| 1486.5 | 30.6 | 0.45 | 66 | 21 |
| 1498.7 | 34.9 | 0.52 | 66 | 14 |
| 1499.9 | 30.6 | 0.56 | 91 | 34 |
| 1502.8 | 28.8 | 0.44 | 75 | 31 |
| 1502.9 | 16.8 | 0.66 | 68 | 2 |
| 1508.9 | 16.8 | 0.48 | 57 | 9 |
| 1511.7 | 38.4 | 0.55 | 80 | 24 |
| 1518 | 26.8 | 0.45 | 93 | 48 |
| 1520.7 | 27.9 | 0.45 | 64 | 19 |
| 1527.9 | 34.7 | 0.43 | 73 | 29 |
| 1529.7 | 54.1 | −0.48 | 36 | 84 |
| 1535 | 28.3 | 0.61 | 73 | 12 |
| 1537.9 | 31.5 | 0.43 | 70 | 28 |
| 1540.7 | 29.8 | 0.5 | 66 | 16 |
| 1542.5 | 27.2 | 0.4 | 52 | 12 |
| 1548.3 | 31.1 | 0.46 | 89 | 43 |
| 1556.8 | 33.7 | 0.59 | 89 | 29 |
| 1567 | 31.9 | 0.45 | 86 | 41 |
| 1567.6 | 53.9 | −0.53 | 2 | 55 |
| 1568.6 | 34.3 | 0.41 | 70 | 29 |
| 1573.8 | 40.4 | 0.44 | 89 | 45 |
| 1574.8 | 33.9 | 0.41 | 61 | 21 |
| 1582.9 | 27.8 | 0.51 | 61 | 10 |
| 1588.4 | 47.9 | −0.61 | 11 | 72 |
| 1596.9 | 34 | 0.64 | 86 | 22 |
| 1604.3 | 21.6 | 0.5 | 64 | 14 |
| 1604.7 | 38.1 | 0.42 | 73 | 31 |
| 1605.7 | 53.3 | −0.4 | 34 | 74 |
| 1611.7 | 53.2 | −0.48 | 36 | 84 |
| 1612.8 | 36.8 | 0.58 | 91 | 33 |
| 1622 | 19.2 | 0.51 | 91 | 40 |
| 1633.8 | 24.6 | 0.42 | 75 | 33 |
| 1644 | 18.8 | 0.46 | 55 | 9 |
| 1652.3 | 28.6 | 0.44 | 84 | 40 |
| 1669.9 | 33.4 | 0.54 | 75 | 21 |
| 1676 | 25.3 | 0.52 | 64 | 12 |
| 1681.6 | 40 | 0.51 | 82 | 31 |
| 1686.8 | 38.2 | 0.67 | 91 | 24 |
| 1690.8 | 25.5 | 0.44 | 75 | 31 |
| 1692.5 | 44.2 | −0.41 | 39 | 79 |
| 1699.1 | 41.9 | 0.62 | 86 | 24 |
| 1711 | 43.3 | −0.45 | 20 | 66 |
| 1718.5 | 22.6 | 0.57 | 66 | 9 |
| 1726 | 36.3 | 0.62 | 70 | 9 |
| 1729.2 | 26 | 0.57 | 70 | 14 |
| 1732 | 51.6 | −0.41 | 36 | 78 |
| 1739.8 | 35.7 | 0.45 | 86 | 41 |
| 1746.2 | 46.2 | −0.59 | 25 | 84 |
| 1747.7 | 50.8 | −0.52 | 5 | 57 |
| 1752.9 | 39.9 | 0.46 | 68 | 22 |
| 1763 | 24.4 | 0.57 | 80 | 22 |
| 1770.4 | 45.4 | 0.4 | 89 | 48 |
| 1777.6 | 28.6 | 0.53 | 70 | 17 |
| 1793.6 | 28.3 | 0.49 | 55 | 5 |
| 1804.7 | 34 | 0.45 | 100 | 55 |
| 1808.1 | 45.6 | 0.49 | 55 | 5 |
| 1810.1 | 31.8 | 0.45 | 64 | 19 |
| 1811.3 | 31.3 | 0.55 | 93 | 38 |
| 1813.4 | 54.7 | −0.47 | 7 | 53 |
| 1815.2 | 27.7 | 0.5 | 66 | 16 |
| 1819.9 | 24.1 | 0.5 | 64 | 14 |
| 1820.1 | 31.8 | 0.48 | 91 | 43 |
| 1821.2 | 18.2 | 0.52 | 57 | 5 |
| 1822.9 | 40.7 | −0.6 | 23 | 83 |
| 1824.3 | 37 | −0.52 | 27 | 79 |
| 1826.1 | 21.8 | 0.45 | 59 | 14 |
| 1831.9 | 41.5 | 0.5 | 59 | 9 |
| 1847.8 | 57 | −0.66 | 27 | 93 |
| 1851.2 | 31.6 | 0.48 | 86 | 38 |
| 1853 | 31.2 | 0.59 | 82 | 22 |
| 1853.6 | 46.7 | 0.47 | 50 | 3 |
| 1854.2 | 28.8 | 0.45 | 52 | 7 |
| 1856.8 | 56.3 | −0.51 | 18 | 69 |
| 1857.1 | 39 | 0.46 | 75 | 29 |
| 1864.6 | 28.6 | 0.65 | 82 | 17 |
| 1867 | 31.8 | 0.6 | 91 | 31 |
| 1883 | 29.1 | −0.49 | 32 | 81 |
| 1885.7 | 57.5 | −0.4 | 55 | 95 |
| 1889.2 | 30.1 | 0.41 | 55 | 14 |
| 1889.8 | 46.4 | −0.58 | 39 | 97 |
| 1891.6 | 32.3 | 0.55 | 77 | 22 |
| 1894.9 | 22 | 0.67 | 86 | 19 |
| 1896.8 | 53.3 | −0.44 | 11 | 55 |
| 1898.7 | 26.5 | 0.45 | 59 | 14 |
| 1904 | 27.5 | 0.48 | 50 | 2 |
| 1913.4 | 30.1 | 0.44 | 55 | 10 |
| 1916.8 | 44.7 | −0.52 | 14 | 66 |
| 1920.7 | 30.6 | 0.46 | 91 | 45 |
| 1933.9 | 32.8 | −0.55 | 36 | 91 |
| 1934.2 | 16.1 | 0.71 | 73 | 2 |
| 1936.5 | 46.6 | −0.42 | 25 | 67 |
| 1936.7 | 32.8 | 0.5 | 80 | 29 |
| 1944.2 | 47 | −0.68 | 18 | 86 |
| 1951.1 | 53 | −0.48 | 16 | 64 |
| 1966.3 | 25.1 | 0.71 | 82 | 10 |
| 1973.7 | 57.1 | −0.5 | 9 | 59 |
| 1977.4 | 42.9 | −0.47 | 41 | 88 |
| 1982.9 | 32.2 | 0.58 | 82 | 24 |
| 1989.3 | 43.7 | 0.69 | 84 | 16 |
| 1990.8 | 47.3 | −0.71 | 11 | 83 |
| 2011.5 | 42 | 0.41 | 70 | 29 |
| 2022.6 | 34.6 | 0.46 | 68 | 22 |
| 2025 | 24.2 | 0.41 | 50 | 9 |
| 2028.4 | 29.9 | 0.55 | 80 | 24 |
| 2030.4 | 31.7 | −0.49 | 32 | 81 |
| 2030.8 | 46.5 | −0.61 | 25 | 86 |
| 2033.5 | 27.5 | 0.46 | 55 | 9 |
| 2042 | 26.4 | 0.47 | 95 | 48 |
| 2042.5 | 40.7 | 0.5 | 70 | 21 |
| 2045.9 | 25.3 | 0.43 | 84 | 41 |
| 2047 | 45.4 | −0.46 | 52 | 98 |
| 2050.8 | 38.2 | 0.47 | 82 | 34 |
| 2065.3 | 20.9 | 0.49 | 52 | 3 |
| 2092 | 26.7 | 0.52 | 66 | 14 |
| 2092.5 | 41.3 | 0.59 | 75 | 16 |
| 2099.2 | 36.9 | 0.58 | 84 | 26 |
| 2103.6 | 26.7 | 0.46 | 89 | 43 |
| 2105.4 | 32.5 | 0.52 | 66 | 14 |
| 2109.3 | 27.9 | 0.47 | 68 | 21 |
| 2117.1 | 57.1 | −0.57 | 20 | 78 |
| 2127.2 | 39.6 | 0.46 | 75 | 29 |
| 2129.5 | 35.1 | −0.58 | 39 | 97 |
| 2140.1 | 26.8 | 0.52 | 64 | 12 |
| 2144.3 | 22 | 0.47 | 75 | 28 |
| 2146.3 | 25.8 | 0.74 | 77 | 3 |
| 2147.2 | 38.4 | 0.43 | 64 | 21 |
| 2152.7 | 29.5 | 0.51 | 70 | 19 |
| 2157.2 | 24.4 | 0.41 | 50 | 9 |
| 2174.4 | 24.6 | 0.46 | 68 | 22 |
| 2178.5 | 21.4 | 0.48 | 57 | 9 |
| 2182.5 | 27.6 | 0.55 | 80 | 24 |
| 2207.2 | 41.9 | 0.41 | 61 | 21 |
| 2210.7 | 25.7 | 0.64 | 86 | 22 |
| 2217.7 | 41.9 | 0.5 | 84 | 34 |
| 2221.1 | 40.7 | −0.5 | 14 | 64 |
| 2223.5 | 22.6 | 0.6 | 68 | 9 |
| 2228.1 | 25.9 | 0.51 | 91 | 40 |
| 2233 | 31.1 | −0.4 | 55 | 95 |
| 2241.1 | 22.7 | 0.49 | 80 | 31 |
| 2290.7 | 36.2 | 0.47 | 52 | 5 |
| 2291.1 | 21.9 | 0.45 | 50 | 5 |
| 2308.9 | 26.2 | 0.41 | 61 | 21 |
| 2312.5 | 22.9 | 0.45 | 57 | 12 |
| 2322.5 | 47.1 | 0.47 | 52 | 5 |
| 2356.3 | 24 | 0.41 | 57 | 16 |
| 2364.4 | 38.9 | 0.49 | 73 | 24 |
| 2370.7 | 27.3 | 0.4 | 52 | 12 |
| 2391.2 | 24.3 | 0.58 | 70 | 12 |
| 2406.4 | 31.8 | 0.43 | 84 | 41 |
| 2409.1 | 41.9 | 0.43 | 84 | 41 |
| 2421 | 28.7 | 0.41 | 70 | 29 |
| 2423.1 | 27.4 | 0.41 | 68 | 28 |
| 2426.5 | 38.5 | 0.58 | 89 | 31 |
| 2427.4 | 24 | 0.53 | 84 | 31 |
| 2432.2 | 38.3 | 0.66 | 80 | 14 |
| 2464 | 47.2 | −0.55 | 7 | 62 |
| 2465 | 22.8 | 0.7 | 75 | 5 |
| 2473.4 | 41.9 | 0.44 | 52 | 9 |
| 2490.7 | 26.7 | 0.43 | 70 | 28 |
| 2493.6 | 24.6 | 0.63 | 77 | 14 |
| 2522.9 | 24.4 | 0.47 | 68 | 21 |
| 2529.2 | 41.4 | −0.47 | 14 | 60 |
| 2535 | 37.7 | 0.42 | 82 | 40 |
| 2540.5 | 25.5 | 0.65 | 75 | 10 |
| 2548.2 | 35.1 | −0.46 | 36 | 83 |
| 2566.4 | 22.2 | 0.5 | 57 | 7 |
| 2568.9 | 26.9 | 0.41 | 70 | 29 |
| 2573.7 | 16.3 | 0.57 | 66 | 9 |
| 2584 | 43.8 | −0.56 | 41 | 97 |
| 2593.4 | 25 | 0.41 | 57 | 16 |
| 2614.1 | 22.5 | 0.42 | 59 | 17 |
| 2619.7 | 22.9 | 0.47 | 50 | 3 |
| 2621.4 | 25.8 | 0.56 | 68 | 12 |
| 2644.1 | 32.5 | −0.48 | 45 | 93 |
| 2660.8 | 27.1 | 0.4 | 59 | 19 |
| 2665.3 | 39.4 | 0.46 | 57 | 10 |
| 2677.6 | 23.6 | 0.58 | 68 | 10 |
| 2698.4 | 32.1 | −0.47 | 32 | 79 |
| 2713.2 | 41.3 | −0.51 | 11 | 62 |
| 2719.9 | 20.2 | 0.49 | 55 | 5 |
| 2752.8 | 25.3 | 0.56 | 82 | 26 |
| 2780.4 | 28.3 | 0.52 | 66 | 14 |
| 2790.3 | 26.8 | 0.46 | 61 | 16 |
| 2793.7 | 36.3 | 0.64 | 80 | 16 |
| 2809.1 | 37.2 | −0.48 | 30 | 78 |
| 2812.5 | 32.8 | 0.46 | 61 | 16 |
| 2830.9 | 33.2 | 0.49 | 68 | 19 |
| 2937.1 | 26.6 | 0.46 | 55 | 9 |
| 2973.7 | 34.9 | −0.58 | 30 | 88 |
| 2978 | 26.3 | −0.49 | 34 | 83 |
| 2990.4 | 33.6 | −0.47 | 20 | 67 |
| 3007.5 | 30.5 | −0.45 | 23 | 67 |
| 3017.7 | 46.8 | −0.42 | 18 | 60 |
| 3057.1 | 56.4 | −0.41 | 43 | 84 |
| 3058.8 | 35.5 | −0.41 | 45 | 86 |
| 3121.4 | 42.5 | −0.43 | 57 | 100 |
| 3137 | 37 | −0.42 | 41 | 83 |
| 3139.4 | 43.7 | −0.53 | 25 | 78 |
| 3152.6 | 38.2 | −0.45 | 55 | 100 |
| 3177.4 | 22.3 | −0.45 | 27 | 72 |
| 3187.7 | 48.6 | −0.4 | 41 | 81 |
| 3209.2 | 34.3 | −0.47 | 50 | 97 |
| 3219.5 | 20.2 | 0.48 | 61 | 14 |
| 3255.8 | 42.9 | −0.48 | 36 | 84 |
| 3262 | 31.5 | −0.52 | 34 | 86 |
| 3281 | 36.8 | −0.66 | 32 | 98 |
| 3282 | 49.4 | −0.4 | 55 | 95 |
| 3290.9 | 36.9 | −0.57 | 36 | 93 |
| 3295.8 | 38.4 | −0.55 | 43 | 98 |
| 3303.2 | 38.6 | −0.57 | 27 | 84 |
| 3308.6 | 21.3 | 0.53 | 57 | 3 |
| 3309.7 | 43.6 | −0.41 | 32 | 72 |
| 3319.3 | 46.2 | −0.45 | 50 | 95 |
| 3333.4 | 23.3 | −0.56 | 32 | 88 |
| 3334.6 | 41.7 | −0.54 | 32 | 86 |
| 3337.4 | 36.2 | −0.45 | 43 | 88 |
| 3343.8 | 43.8 | −0.46 | 45 | 91 |
| 3405.7 | 37.8 | −0.6 | 39 | 98 |
| 3422.5 | 38.7 | −0.58 | 32 | 90 |
| 3436 | 26.4 | −0.5 | 20 | 71 |
| 3479.3 | 48.5 | −0.5 | 50 | 100 |
| 3503.3 | 23.2 | −0.43 | 16 | 59 |
| 3530.9 | 36.8 | −0.54 | 27 | 81 |
| 3583.3 | 25.2 | −0.67 | 23 | 90 |
| 3589.5 | 39.1 | −0.65 | 25 | 90 |
| 3617.4 | 44.8 | −0.4 | 18 | 59 |
| 3631.2 | 33.1 | −0.55 | 16 | 71 |
| 3634.9 | 42.6 | −0.41 | 39 | 79 |
| 3682.4 | 42.8 | −0.47 | 27 | 74 |
| 3686.1 | 32.6 | −0.71 | 11 | 83 |
| 3697.4 | 38.8 | −0.42 | 11 | 53 |
| 3701.8 | 43.4 | −0.63 | 9 | 72 |
| 3707 | 31.9 | −0.69 | 7 | 76 |
| 3719.6 | 44.7 | −0.42 | 41 | 83 |
| 3723.3 | 32.5 | −0.65 | 32 | 97 |
| 3735.8 | 43.9 | −0.5 | 30 | 79 |
| 3760.8 | 25.9 | −0.51 | 18 | 69 |
| 3802.7 | 46.2 | −0.43 | 14 | 57 |
| 3816.7 | 32.2 | −0.52 | 14 | 66 |
| 3852.2 | 36.9 | −0.45 | 36 | 81 |
| 3871.7 | 42.9 | −0.41 | 23 | 64 |
| 3946.9 | 33.1 | −0.54 | 39 | 93 |
| 3969.6 | 31.3 | −0.52 | 34 | 86 |
| 3987 | 30.5 | −0.42 | 55 | 97 |
| 4026.2 | 30.5 | −0.42 | 11 | 53 |
| 4044.7 | 31.2 | −0.57 | 30 | 86 |
| 4055.2 | 24.1 | 0.41 | 57 | 16 |
| 4154.2 | 23.7 | 0.63 | 77 | 14 |
| 4170.6 | 46.1 | −0.45 | 7 | 52 |
| 4183.7 | 26.6 | 0.42 | 52 | 10 |
| 4241.2 | 24.4 | 0.75 | 89 | 14 |
| 4283.1 | 24.3 | 0.55 | 64 | 9 |
| 4290.8 | 41.1 | −0.45 | 43 | 88 |
| 4654.8 | 38.8 | −0.4 | 11 | 52 |
| 4713.7 | 26.9 | 0.63 | 68 | 5 |
| 4748.5 | 25.4 | −0.56 | 39 | 95 |
| 4772.1 | 28.9 | −0.43 | 9 | 52 |
| 4801.2 | 37.5 | −0.49 | 39 | 88 |
| 4827.1 | 27.3 | 0.51 | 52 | 2 |
| 4863.7 | 39.2 | −0.53 | 18 | 71 |
| 5213.8 | 36.8 | −0.43 | 7 | 50 |
| 5229.1 | 39.9 | −0.43 | 9 | 52 |
| 5575.8 | 35.7 | −0.48 | 14 | 62 |
| 6171.5 | 39.6 | −0.5 | 43 | 93 |
| 6212.4 | 30.6 | −0.5 | 9 | 59 |
| 6400.9 | 23.4 | 0.51 | 52 | 2 |
| 7409.9 | 26.2 | 0.49 | 61 | 12 |
| 7556.6 | 26.2 | 0.59 | 75 | 16 |
| 7572.8 | 25.7 | 0.42 | 55 | 12 |
| 8054.8 | 16.7 | 0.63 | 82 | 19 |
| 8341.2 | 16.6 | 0.59 | 66 | 7 |
| 8653.1 | 17.2 | 0.4 | 52 | 12 |
| 8765.9 | 17.6 | 0.56 | 89 | 33 |
| 9060.7 | 23 | 0.46 | 57 | 10 |
| 9076 | 23 | 0.58 | 68 | 10 |
| 9182 | 17.1 | 0.55 | 64 | 9 |
| 9223.1 | 22.8 | 0.64 | 70 | 7 |
| 9335.5 | 17.5 | 0.47 | 50 | 3 |
| 9868.8 | 29.5 | −0.57 | 20 | 78 |
| 9933.5 | 18.4 | 0.47 | 50 | 3 |
| 10046.3 | 18.1 | 0.7 | 89 | 19 |
| 10390.1 | 20.2 | 0.58 | 70 | 12 |
| 10518.8 | 20.9 | 0.56 | 75 | 19 |

TABLE 2

| | | | |
|---|---|---|---|
| molecular | migration | discrimination | frequency [%] |

| weight [Da] | time [min] | factor | FSGS | healthy |
|---|---|---|---|---|
| 803.4 | 35.2 | 0.44 | 60 | 16 |
| 807.3 | 35.4 | 0.41 | 50 | 9 |
| 809.3 | 25.2 | 0.5 | 50 | 0 |
| 817.2 | 25.6 | 0.47 | 50 | 3 |
| 830.5 | 25.3 | 0.68 | 80 | 12 |
| 836.5 | 35 | 0.54 | 70 | 16 |
| 866.4 | 37.9 | 0.64 | 80 | 16 |
| 870.4 | 33.9 | 0.43 | 50 | 7 |
| 874.4 | 49.7 | 0.57 | 60 | 3 |
| 907.4 | 27.5 | 0.57 | 60 | 3 |
| 909.4 | 40.3 | 0.57 | 60 | 3 |
| 915.4 | 35 | 0.48 | 50 | 2 |
| 925.4 | 50.7 | 0.56 | 70 | 14 |
| 926.5 | 36.1 | −0.46 | 30 | 76 |
| 939.6 | 33.1 | 0.61 | 70 | 9 |
| 943.5 | 30 | 0.42 | 70 | 28 |
| 946.5 | 46.8 | −0.54 | 30 | 84 |
| 950.5 | 36 | 0.75 | 80 | 5 |
| 952.5 | 32 | 0.58 | 70 | 12 |
| 953.6 | 34.5 | −0.52 | 20 | 72 |
| 956.4 | 49.5 | −0.5 | 10 | 60 |
| 958.5 | 32.5 | 0.48 | 70 | 22 |
| 974.5 | 27.6 | 0.41 | 60 | 19 |
| 978.5 | 35 | 0.48 | 50 | 2 |
| 980.6 | 34.6 | −0.58 | 30 | 88 |
| 981.6 | 37.4 | −0.47 | 50 | 97 |
| 982.5 | 31.9 | 0.7 | 80 | 10 |
| 988.6 | 49.9 | −0.47 | 10 | 57 |
| 990.6 | 32 | 0.41 | 60 | 19 |
| 994.5 | 33.3 | 0.46 | 70 | 24 |
| 995.6 | 36.4 | 0.48 | 70 | 22 |
| 1000.5 | 34 | −0.55 | 40 | 95 |
| 1002.6 | 38.5 | 0.53 | 60 | 7 |
| 1005.5 | 35 | 0.54 | 70 | 16 |
| 1006.4 | 35.7 | −0.58 | 20 | 78 |
| 1008.5 | 34.4 | −0.63 | 30 | 93 |
| 1010.6 | 30.6 | −0.53 | 30 | 83 |
| 1010.6 | 50.8 | −0.64 | 0 | 64 |
| 1013.4 | 39.3 | −0.52 | 0 | 52 |
| 1015.6 | 38.2 | 0.55 | 60 | 5 |
| 1021.5 | 32.1 | 0.45 | 50 | 5 |
| 1028.6 | 37.8 | −0.58 | 40 | 98 |
| 1033.6 | 39.1 | 0.71 | 80 | 9 |
| 1041.5 | 51.6 | −0.4 | 20 | 60 |
| 1046.6 | 38.6 | −0.77 | 20 | 97 |
| 1049.5 | 39.9 | 0.45 | 50 | 5 |
| 1055.6 | 36.4 | 0.51 | 60 | 9 |
| 1071.5 | 38.7 | 0.48 | 50 | 2 |
| 1073.6 | 34.7 | 0.64 | 80 | 16 |
| 1075.6 | 29 | −0.44 | 20 | 64 |
| 1090.5 | 36.2 | 0.42 | 70 | 28 |
| 1106.6 | 19.6 | 0.41 | 50 | 9 |
| 1108.6 | 29.8 | 0.4 | 80 | 40 |
| 1110.4 | 46.9 | −0.41 | 30 | 71 |
| 1121.6 | 42.3 | −0.62 | 0 | 62 |
| 1126.5 | 42.7 | 0.51 | 60 | 9 |
| 1135.6 | 42.7 | −0.66 | 0 | 66 |
| 1138.6 | 39.3 | 0.63 | 80 | 17 |
| 1139.6 | 32.2 | 0.53 | 60 | 7 |
| 1141.6 | 38 | −0.57 | 10 | 67 |
| 1157.6 | 28.5 | 0.51 | 80 | 29 |
| 1157.6 | 16.9 | 0.5 | 50 | 0 |
| 1159.6 | 39 | −0.61 | 20 | 81 |
| 1164.6 | 38.7 | 0.46 | 60 | 14 |
| 1171.6 | 32.8 | 0.48 | 50 | 2 |
| 1182.6 | 47.2 | −0.66 | 0 | 66 |
| 1186.6 | 32 | 0.47 | 80 | 33 |
| 1191.6 | 50.5 | −0.71 | 20 | 91 |
| 1191.8 | 18.3 | 0.5 | 60 | 10 |
| 1192.6 | 40.9 | 0.46 | 70 | 24 |
| 1203.7 | 24.7 | −0.47 | 10 | 57 |
| 1209.6 | 50.5 | −0.5 | 40 | 90 |
| 1211.6 | 31.3 | 0.64 | 80 | 16 |
| 1212.7 | 30.6 | 0.61 | 70 | 9 |
| 1219.6 | 37.3 | 0.59 | 80 | 21 |
| 1220.6 | 30.2 | 0.57 | 90 | 33 |
| 1224.7 | 33.6 | −0.5 | 50 | 100 |
| 1225.7 | 41.3 | 0.55 | 60 | 5 |
| 1235.6 | 41.4 | −0.4 | 60 | 100 |
| 1236.7 | 34.8 | 0.44 | 70 | 26 |
| 1237.7 | 41.6 | −0.47 | 20 | 67 |
| 1246.7 | 30.5 | −0.46 | 20 | 66 |
| 1258.7 | 20.9 | 0.5 | 60 | 10 |
| 1263.7 | 38.6 | 0.65 | 70 | 5 |
| 1268.6 | 53.7 | −0.52 | 0 | 52 |
| 1269.7 | 39.8 | 0.51 | 70 | 19 |
| 1270.5 | 52.5 | −0.55 | 0 | 55 |
| 1270.6 | 25.7 | 0.55 | 60 | 5 |
| 1274.6 | 38 | 0.56 | 70 | 14 |
| 1279.7 | 38.3 | 0.44 | 60 | 16 |
| 1280.6 | 51.9 | −0.52 | 10 | 62 |
| 1288.6 | 46.1 | −0.44 | 40 | 84 |
| 1292.5 | 53 | −0.66 | 20 | 86 |
| 1294.6 | 54.4 | 0.56 | 70 | 14 |
| 1302.7 | 31.8 | 0.57 | 90 | 33 |
| 1303.6 | 40.7 | −0.45 | 10 | 55 |
| 1304.8 | 24.6 | 0.5 | 50 | 0 |
| 1305.9 | 33.4 | 0.6 | 70 | 10 |
| 1308.6 | 53.6 | −0.54 | 30 | 84 |
| 1309.8 | 38.9 | 0.41 | 50 | 9 |
| 1310.7 | 52.5 | 0.5 | 50 | 0 |
| 1311.8 | 31.5 | 0.51 | 70 | 19 |
| 1325.5 | 35.2 | 0.44 | 70 | 26 |
| 1333.8 | 38.8 | 0.63 | 80 | 17 |
| 1335.7 | 39.2 | 0.68 | 90 | 22 |
| 1338.7 | 47.2 | 0.78 | 80 | 2 |
| 1338.7 | 29.6 | 0.7 | 80 | 10 |
| 1350.7 | 50.3 | −0.5 | 0 | 50 |
| 1353.7 | 39.3 | −0.5 | 40 | 90 |
| 1354.8 | 45.6 | 0.52 | 90 | 38 |
| 1367.7 | 26.2 | 0.41 | 50 | 9 |
| 1371.7 | 39.9 | 0.67 | 70 | 3 |
| 1371.8 | 19.3 | 0.64 | 90 | 26 |
| 1377.7 | 25.4 | 0.67 | 70 | 3 |
| 1383.7 | 52.7 | −0.53 | 0 | 53 |
| 1386 | 24.4 | 0.41 | 50 | 9 |
| 1389.8 | 19.5 | 0.56 | 90 | 34 |
| 1390.7 | 41.1 | 0.71 | 90 | 19 |
| 1394.3 | 48.1 | 0.48 | 60 | 12 |
| 1398.9 | 30.5 | 0.56 | 70 | 14 |
| 1401.8 | 46.2 | −0.62 | 0 | 62 |
| 1403.8 | 34.4 | 0.57 | 60 | 3 |
| 1405.9 | 17.3 | 0.6 | 70 | 10 |
| 1408.9 | 26.8 | 0.5 | 60 | 10 |
| 1411.2 | 45.4 | 0.41 | 70 | 29 |
| 1414.6 | 38.1 | 0.76 | 100 | 24 |
| 1414.7 | 26.6 | 0.48 | 50 | 2 |
| 1419.8 | 39.7 | 0.41 | 70 | 29 |
| 1424.9 | 35.4 | −0.61 | 10 | 71 |
| 1426.8 | 38.7 | 0.41 | 50 | 9 |
| 1434.8 | 41.2 | 0.48 | 50 | 2 |
| 1442.8 | 33.3 | 0.69 | 90 | 21 |
| 1444.6 | 37.8 | 0.61 | 90 | 29 |
| 1465.9 | 28.8 | 0.53 | 60 | 7 |
| 1472.1 | 31.2 | 0.46 | 60 | 14 |
| 1474.9 | 16.9 | 0.58 | 70 | 12 |
| 1482 | 30.4 | 0.42 | 70 | 28 |
| 1484 | 30.4 | 0.49 | 80 | 31 |
| 1487.7 | 41.4 | −0.41 | 50 | 91 |
| 1490.9 | 53.4 | −0.41 | 30 | 71 |
| 1493.7 | 33.7 | 0.41 | 70 | 29 |
| 1498.7 | 34.9 | 0.46 | 60 | 14 |
| 1499.9 | 30.6 | 0.56 | 90 | 34 |
| 1501.1 | 29.5 | 0.45 | 50 | 5 |
| 1502.9 | 16.8 | 0.68 | 70 | 2 |
| 1508.9 | 16.8 | 0.51 | 60 | 9 |
| 1511.7 | 38.4 | 0.56 | 80 | 24 |
| 1517.6 | 54.8 | 0.48 | 60 | 12 |
| 1518 | 26.8 | 0.52 | 100 | 48 |
| 1518.9 | 42.5 | 0.48 | 60 | 12 |
| 1520.7 | 27.9 | 0.41 | 60 | 19 |
| 1527.9 | 34.7 | 0.41 | 70 | 29 |
| 1529.7 | 54.1 | −0.44 | 40 | 84 |
| 1535 | 28.3 | 0.68 | 80 | 12 |
| 1536.6 | 35 | 0.46 | 60 | 14 |
| 1537.9 | 31.5 | 0.52 | 80 | 28 |
| 1540.7 | 29.8 | 0.44 | 60 | 16 |
| 1547 | 38.3 | 0.5 | 90 | 40 |
| 1548.3 | 31.1 | 0.47 | 90 | 43 |
| 1553.2 | 54.4 | 0.48 | 60 | 12 |
| 1556.8 | 33.7 | 0.51 | 80 | 29 |
| 1567 | 31.9 | 0.49 | 90 | 41 |
| 1567.6 | 53.9 | −0.55 | 0 | 55 |
| 1588.4 | 47.9 | −0.62 | 10 | 72 |
| 1589.7 | 54.3 | −0.44 | 40 | 84 |
| 1591.6 | 32.6 | 0.53 | 60 | 7 |
| 1596.9 | 34 | 0.58 | 80 | 22 |
| 1598.7 | 28.5 | 0.48 | 60 | 12 |
| 1604.3 | 21.6 | 0.56 | 70 | 14 |
| 1604.7 | 38.1 | 0.49 | 80 | 31 |
| 1605.7 | 53.3 | −0.44 | 30 | 74 |
| 1607.7 | 41 | 0.48 | 60 | 12 |
| 1611.7 | 53.2 | −0.54 | 30 | 84 |
| 1612.8 | 36.8 | 0.47 | 80 | 33 |
| 1619.7 | 53.9 | −0.52 | 10 | 62 |
| 1622 | 19.2 | 0.6 | 100 | 40 |
| 1644 | 18.8 | 0.41 | 50 | 9 |
| 1669.9 | 33.4 | 0.59 | 80 | 21 |
| 1671.3 | 25.4 | 0.42 | 80 | 38 |
| 1681.6 | 40 | 0.59 | 90 | 31 |
| 1686.8 | 38.2 | 0.66 | 90 | 24 |
| 1692.5 | 44.2 | −0.59 | 20 | 79 |
| 1695.1 | 23.6 | 0.48 | 60 | 12 |
| 1699.1 | 41.9 | 0.46 | 70 | 24 |
| 1711 | 43.3 | −0.46 | 20 | 66 |
| 1713.4 | 24.6 | 0.41 | 60 | 19 |
| 1718.5 | 22.6 | 0.41 | 50 | 9 |
| 1723.3 | 26.5 | 0.43 | 60 | 17 |
| 1726 | 36.3 | 0.61 | 70 | 9 |
| 1729.1 | 37.7 | 0.41 | 50 | 9 |
| 1729.2 | 26 | 0.66 | 80 | 14 |
| 1732 | 51.6 | −0.48 | 30 | 78 |
| 1739.8 | 35.7 | 0.49 | 90 | 41 |
| 1746.2 | 46.2 | −0.54 | 30 | 84 |
| 1747.7 | 50.8 | −0.47 | 10 | 57 |
| 1751.4 | 40.8 | 0.42 | 80 | 38 |
| 1752.9 | 39.9 | 0.48 | 70 | 22 |
| 1763 | 24.4 | 0.68 | 90 | 22 |
| 1770.4 | 45.4 | 0.52 | 100 | 48 |
| 1772.6 | 28.5 | 0.44 | 60 | 16 |
| 1776.1 | 43.6 | −0.43 | 40 | 83 |
| 1777.6 | 28.6 | 0.73 | 90 | 17 |
| 1783.4 | 29.2 | 0.51 | 70 | 19 |
| 1784.9 | 31.4 | 0.47 | 50 | 3 |
| 1786.9 | 35.9 | 0.53 | 70 | 17 |
| 1788.6 | 31 | 0.49 | 90 | 41 |
| 1792.4 | 42.3 | 0.59 | 80 | 21 |
| 1794.9 | 40.4 | 0.4 | 80 | 40 |
| 1804.7 | 34 | 0.45 | 100 | 55 |
| 1808.1 | 45.6 | 0.55 | 60 | 5 |
| 1810.1 | 31.8 | 0.41 | 60 | 19 |
| 1811.3 | 31.3 | 0.52 | 90 | 38 |
| 1813.4 | 54.7 | −0.43 | 10 | 53 |
| 1815.1 | 39.3 | 0.41 | 60 | 19 |
| 1815.2 | 27.7 | 0.44 | 60 | 16 |
| 1820.1 | 31.8 | 0.57 | 100 | 43 |
| 1821.2 | 18.2 | 0.45 | 50 | 5 |
| 1821.5 | 42.1 | 0.46 | 70 | 24 |
| 1822.9 | 40.7 | −0.73 | 10 | 83 |
| 1824.3 | 37 | −0.59 | 20 | 79 |
| 1831.7 | 25.2 | 0.43 | 50 | 7 |
| 1831.9 | 41.5 | 0.61 | 70 | 9 |
| 1844.2 | 34.6 | 0.62 | 90 | 28 |
| 1847.8 | 57 | −0.73 | 20 | 93 |
| 1849.6 | 37.2 | −0.41 | 40 | 81 |
| 1851.2 | 31.6 | 0.52 | 90 | 38 |
| 1853 | 31.2 | 0.58 | 80 | 22 |
| 1853.4 | 18.3 | 0.5 | 50 | 0 |
| 1853.6 | 46.7 | 0.57 | 60 | 3 |
| 1854.2 | 28.8 | 0.43 | 50 | 7 |
| 1856.8 | 56.3 | −0.59 | 10 | 69 |
| 1857.1 | 39 | 0.51 | 80 | 29 |
| 1864.6 | 28.6 | 0.43 | 60 | 17 |
| 1867 | 31.8 | 0.49 | 80 | 31 |
| 1870.5 | 16.1 | 0.45 | 50 | 5 |
| 1871.7 | 43.2 | 0.65 | 70 | 5 |
| 1881.4 | 34.3 | 0.47 | 80 | 33 |
| 1889.2 | 30.1 | 0.56 | 70 | 14 |
| 1889.8 | 46.4 | −0.67 | 30 | 97 |
| 1891.6 | 32.3 | 0.58 | 80 | 22 |
| 1894.9 | 22 | 0.71 | 90 | 19 |
| 1894.9 | 56 | −0.46 | 20 | 66 |
| 1898.7 | 26.5 | 0.46 | 60 | 14 |
| 1900.7 | 30.4 | −0.63 | 20 | 83 |
| 1902.8 | 33.1 | 0.53 | 70 | 17 |
| 1904 | 27.5 | 0.48 | 50 | 2 |
| 1904.3 | 43.2 | 0.41 | 50 | 9 |
| 1916.8 | 44.7 | −0.46 | 20 | 66 |
| 1920.5 | 46.1 | 0.44 | 60 | 16 |
| 1920.7 | 30.6 | 0.45 | 90 | 45 |
| 1925.3 | 52.5 | 0.57 | 90 | 33 |
| 1928.4 | 33.2 | 0.48 | 70 | 22 |
| 1933.9 | 32.8 | −0.41 | 50 | 91 |
| 1934.2 | 16.1 | 0.78 | 80 | 2 |
| 1936.5 | 46.6 | −0.47 | 20 | 67 |
| 1936.7 | 32.8 | 0.51 | 80 | 29 |
| 1944.2 | 47 | −0.66 | 20 | 86 |
| 1950.9 | 34.5 | 0.59 | 90 | 31 |
| 1951.1 | 53 | −0.54 | 10 | 64 |
| 1966.3 | 25.1 | 0.6 | 70 | 10 |
| 1973.7 | 57.1 | −0.49 | 10 | 59 |
| 1977.4 | 42.9 | −0.48 | 40 | 88 |
| 1982.9 | 32.2 | 0.46 | 70 | 24 |
| 1989.3 | 43.7 | 0.54 | 70 | 16 |
| 1990.8 | 47.3 | −0.83 | 0 | 83 |
| 1999.4 | 35.6 | 0.4 | 80 | 40 |
| 2005.3 | 39.6 | 0.49 | 80 | 31 |
| 2013.8 | 45.3 | −0.47 | 20 | 67 |
| 2022.6 | 34.6 | 0.48 | 70 | 22 |
| 2028.4 | 29.9 | 0.66 | 90 | 24 |
| 2030.8 | 46.5 | −0.56 | 30 | 86 |
| 2033.5 | 27.5 | 0.51 | 60 | 9 |
| 2042 | 26.4 | 0.52 | 100 | 48 |
| 2042.5 | 40.7 | 0.49 | 70 | 21 |
| 2047 | 45.4 | −0.48 | 50 | 98 |
| 2048.2 | 33.1 | −0.6 | 40 | 100 |
| 2057.2 | 36.3 | 0.43 | 100 | 57 |
| 2063 | 24.3 | 0.6 | 60 | 0 |
| 2065.3 | 20.9 | 0.57 | 60 | 3 |
| 2077.3 | 35.8 | −0.42 | 10 | 52 |
| 2092.5 | 41.3 | 0.44 | 60 | 16 |
| 2099.2 | 36.9 | 0.54 | 80 | 26 |
| 2105.4 | 32.5 | 0.56 | 70 | 14 |
| 2114.9 | 42 | 0.48 | 70 | 22 |
| 2117.1 | 57.1 | −0.68 | 10 | 78 |
| 2121 | 26.9 | 0.61 | 80 | 19 |
| 2127.2 | 39.6 | 0.61 | 90 | 29 |
| 2140.1 | 26.8 | 0.48 | 60 | 12 |
| 2144.3 | 22 | 0.42 | 70 | 28 |
| 2146.3 | 25.8 | 0.57 | 60 | 3 |
| 2147.2 | 38.4 | 0.69 | 90 | 21 |
| 2152.7 | 29.5 | 0.51 | 70 | 19 |
| 2163.4 | 27.6 | 0.6 | 70 | 10 |
| 2174.4 | 24.6 | 0.58 | 80 | 22 |
| 2178.5 | 21.4 | 0.41 | 50 | 9 |
| 2178.7 | 47.5 | −0.4 | 20 | 60 |
| 2182.5 | 27.6 | 0.46 | 70 | 24 |
| 2200.3 | 47 | 0.5 | 50 | 0 |
| 2210.7 | 25.7 | 0.58 | 80 | 22 |
| 2212.9 | 46.3 | −0.44 | 40 | 84 |
| 2221.1 | 40.7 | −0.44 | 20 | 64 |
| 2223.5 | 22.6 | 0.41 | 50 | 9 |
| 2228.1 | 25.9 | 0.5 | 90 | 40 |
| 2233 | 31.1 | −0.65 | 30 | 95 |
| 2257.2 | 46.6 | −0.4 | 60 | 100 |
| 2258.9 | 33.6 | 0.62 | 100 | 38 |
| 2290.7 | 36.2 | 0.45 | 50 | 5 |
| 2291.1 | 21.9 | 0.45 | 50 | 5 |
| 2322.5 | 47.1 | 0.55 | 60 | 5 |
| 2334.2 | 41.2 | 0.45 | 50 | 5 |
| 2338.2 | 40.4 | 0.53 | 60 | 7 |
| 2367.7 | 43.2 | −0.43 | 40 | 83 |
| 2375.2 | 36.5 | 0.42 | 80 | 38 |
| 2391.2 | 24.3 | 0.48 | 60 | 12 |
| 2409.1 | 41.9 | 0.49 | 90 | 41 |
| 2423.1 | 27.4 | 0.42 | 70 | 28 |
| 2426.5 | 38.5 | 0.49 | 80 | 31 |
| 2427.4 | 24 | 0.59 | 90 | 31 |
| 2429.9 | 39.3 | −0.46 | 30 | 76 |
| 2432.2 | 38.3 | 0.76 | 90 | 14 |
| 2435 | 21.6 | 0.48 | 50 | 2 |
| 2438.3 | 52.6 | 0.46 | 60 | 14 |
| 2443.4 | 31.9 | −0.44 | 40 | 84 |
| 2464 | 47.2 | −0.52 | 10 | 62 |
| 2465 | 22.8 | 0.65 | 70 | 5 |
| 2473.4 | 41.9 | 0.51 | 60 | 9 |
| 2490.5 | 43 | 0.43 | 60 | 17 |
| 2490.7 | 26.7 | 0.42 | 70 | 28 |
| 2493.6 | 24.6 | 0.66 | 80 | 14 |
| 2529.2 | 41.4 | −0.5 | 10 | 60 |
| 2536.6 | 24.8 | 0.51 | 60 | 9 |
| 2540.5 | 25.5 | 0.5 | 60 | 10 |
| 2542.6 | 42.1 | 0.41 | 50 | 9 |
| 2568.9 | 26.9 | 0.41 | 70 | 29 |
| 2570.5 | 57.1 | −0.58 | 20 | 78 |
| 2573.7 | 16.3 | 0.51 | 60 | 9 |
| 2584 | 43.8 | −0.67 | 30 | 97 |
| 2591.5 | 37.7 | −0.4 | 10 | 50 |
| 2592.5 | 56.6 | −0.42 | 20 | 62 |
| 2593.4 | 25 | 0.44 | 60 | 16 |
| 2608.6 | 37.6 | 0.43 | 90 | 47 |
| 2614.1 | 22.5 | 0.53 | 70 | 17 |
| 2619.7 | 22.9 | 0.47 | 50 | 3 |
| 2621.4 | 25.8 | 0.48 | 60 | 12 |
| 2627.4 | 44.8 | −0.41 | 50 | 91 |
| 2630.6 | 41.7 | 0.41 | 60 | 19 |
| 2646.7 | 21.9 | 0.53 | 60 | 7 |
| 2660.8 | 27.1 | 0.51 | 70 | 19 |
| 2677.6 | 23.6 | 0.5 | 60 | 10 |
| 2679.5 | 35 | −0.5 | 50 | 100 |
| 2687.4 | 41.9 | −0.4 | 60 | 100 |
| 2690.3 | 24.8 | 0.45 | 50 | 5 |
| 2713.2 | 41.3 | −0.52 | 10 | 62 |
| 2720.6 | 39.5 | 0.47 | 50 | 3 |
| 2752.8 | 25.3 | 0.44 | 70 | 26 |
| 2767.4 | 31.4 | −0.55 | 40 | 95 |
| 2780.4 | 28.3 | 0.46 | 60 | 14 |
| 2793.7 | 36.3 | 0.64 | 80 | 16 |
| 2825.4 | 36.5 | −0.48 | 50 | 98 |
| 2830.9 | 33.2 | 0.51 | 70 | 19 |
| 2841.6 | 37.1 | −0.42 | 30 | 72 |
| 2854.4 | 43.8 | −0.47 | 50 | 97 |
| 2883.6 | 28.9 | 0.63 | 80 | 17 |
| 2892.2 | 32.1 | 0.51 | 80 | 29 |
| 2898.5 | 42.3 | −0.44 | 40 | 84 |
| 2902.9 | 42.1 | 0.48 | 60 | 12 |
| 2911.7 | 36.8 | −0.54 | 30 | 84 |
| 2918 | 42.2 | −0.54 | 20 | 74 |
| 2937.1 | 26.6 | 0.51 | 60 | 9 |
| 2945.1 | 22.6 | 0.41 | 50 | 9 |
| 2973.7 | 34.9 | −0.58 | 30 | 88 |
| 2978 | 26.3 | −0.53 | 30 | 83 |
| 2986.9 | 47.3 | −0.41 | 40 | 81 |
| 2990.4 | 33.6 | −0.57 | 10 | 67 |
| 3012.1 | 39.4 | −0.4 | 60 | 100 |
| 3017.7 | 46.8 | −0.5 | 10 | 60 |
| 3022.8 | 33.8 | −0.57 | 40 | 97 |
| 3038.1 | 33.7 | −0.51 | 30 | 81 |
| 3047.7 | 35.9 | −0.49 | 30 | 79 |
| 3057.1 | 56.4 | −0.54 | 30 | 84 |
| 3058.8 | 35.5 | −0.46 | 40 | 86 |
| 3080.2 | 31.7 | −0.4 | 20 | 60 |
| 3098.8 | 42.6 | −0.4 | 60 | 100 |
| 3108.8 | 44.7 | −0.45 | 50 | 95 |
| 3121.4 | 42.5 | −0.5 | 50 | 100 |
| 3137 | 37 | −0.53 | 30 | 83 |
| 3139.4 | 43.7 | −0.58 | 20 | 78 |
| 3152.6 | 38.2 | −0.4 | 60 | 100 |
| 3158.8 | 43.3 | −0.51 | 40 | 91 |
| 3166.2 | 41.2 | −0.43 | 50 | 93 |
| 3177.4 | 22.3 | −0.52 | 20 | 72 |
| 3187.7 | 48.6 | −0.41 | 40 | 81 |
| 3209.2 | 34.3 | −0.67 | 30 | 97 |
| 3255.8 | 42.9 | −0.64 | 20 | 84 |
| 3262 | 31.5 | −0.46 | 40 | 86 |
| 3281 | 36.8 | −0.78 | 20 | 98 |
| 3290.9 | 36.9 | −0.53 | 40 | 93 |
| 3293.2 | 54.2 | −0.47 | 50 | 97 |
| 3295.8 | 38.4 | −0.48 | 50 | 98 |
| 3303.2 | 38.6 | −0.54 | 30 | 84 |
| 3308.6 | 21.3 | 0.57 | 60 | 3 |
| 3315.1 | 54.1 | −0.61 | 10 | 71 |
| 3319.3 | 46.2 | −0.45 | 50 | 95 |
| 3322.8 | 27.3 | −0.48 | 30 | 78 |
| 3333.4 | 23.3 | −0.68 | 20 | 88 |
| 3334.6 | 41.7 | −0.56 | 30 | 86 |
| 3337.4 | 36.2 | −0.58 | 30 | 88 |
| 3343.8 | 43.8 | −0.61 | 30 | 91 |
| 3360.1 | 44.3 | −0.4 | 60 | 100 |
| 3376.2 | 45.2 | −0.47 | 50 | 97 |
| 3402.4 | 33.8 | −0.48 | 50 | 98 |
| 3405.7 | 37.8 | −0.58 | 40 | 98 |
| 3421.8 | 21.1 | 0.47 | 50 | 3 |
| 3422.5 | 38.7 | −0.6 | 30 | 90 |
| 3436 | 26.4 | −0.61 | 10 | 71 |
| 3503.3 | 23.2 | −0.49 | 10 | 59 |
| 3530.9 | 36.8 | −0.51 | 30 | 81 |
| 3547.3 | 38.5 | −0.4 | 10 | 50 |
| 3583.3 | 25.2 | −0.9 | 0 | 90 |
| 3589.5 | 39.1 | −0.8 | 10 | 90 |
| 3631.2 | 33.1 | −0.51 | 20 | 71 |
| 3634.9 | 42.6 | −0.49 | 30 | 79 |
| 3682.4 | 42.8 | −0.44 | 30 | 74 |
| 3686.1 | 32.6 | −0.73 | 10 | 83 |
| 3697.4 | 38.8 | −0.43 | 10 | 53 |
| 3701.8 | 43.4 | −0.62 | 10 | 72 |
| 3707 | 31.9 | −0.76 | 0 | 76 |
| 3719.6 | 44.7 | −0.43 | 40 | 83 |
| 3723.3 | 32.5 | −0.77 | 20 | 97 |
| 3760.8 | 25.9 | −0.49 | 20 | 69 |
| 3802.7 | 46.2 | −0.47 | 10 | 57 |
| 3816.7 | 32.2 | −0.56 | 10 | 66 |
| 3852.2 | 36.9 | −0.41 | 40 | 81 |
| 3871.7 | 42.9 | −0.54 | 10 | 64 |
| 3946.9 | 33.1 | −0.63 | 30 | 93 |
| 3955.9 | 23.6 | 0.41 | 50 | 9 |
| 3969.6 | 31.3 | −0.46 | 40 | 86 |
| 3987 | 30.5 | −0.47 | 50 | 97 |
| 4026.2 | 30.5 | −0.53 | 0 | 53 |
| 4044.7 | 31.2 | −0.56 | 30 | 86 |
| 4055.2 | 24.1 | 0.54 | 70 | 16 |
| 4154.2 | 23.7 | 0.66 | 80 | 14 |
| 4170.6 | 46.1 | −0.42 | 10 | 52 |
| 4241.2 | 24.4 | 0.66 | 80 | 14 |
| 4283.1 | 24.3 | 0.41 | 50 | 9 |
| 4306.5 | 41.4 | −0.4 | 20 | 60 |
| 4335.8 | 27.1 | 0.43 | 50 | 7 |
| 4527.7 | 26 | 0.48 | 50 | 2 |
| 4594.6 | 20.6 | 0.45 | 50 | 5 |
| 4654.8 | 38.8 | −0.42 | 10 | 52 |
| 4713.7 | 26.9 | 0.65 | 70 | 5 |
| 4748.5 | 25.4 | −0.45 | 50 | 95 |
| 4772.1 | 28.9 | −0.42 | 10 | 52 |
| 4863.7 | 39.2 | −0.51 | 20 | 71 |
| 5213.8 | 36.8 | −0.4 | 10 | 50 |
| 5229.1 | 39.9 | −0.52 | 0 | 52 |
| 5428.4 | 33.5 | −0.44 | 30 | 74 |
| 5575.8 | 35.7 | −0.52 | 10 | 62 |
| 5845.8 | 21.8 | 0.5 | 50 | 0 |
| 6171.5 | 39.6 | −0.63 | 30 | 93 |
| 6212.4 | 30.6 | −0.49 | 10 | 59 |
| 6238.6 | 30.9 | −0.56 | 20 | 76 |
| 7556.6 | 26.2 | 0.44 | 60 | 16 |
| 7885.4 | 20.9 | 0.45 | 50 | 5 |
| 8054.8 | 16.7 | 0.61 | 80 | 19 |
| 8341.2 | 16.6 | 0.53 | 60 | 7 |
| 8765.9 | 17.6 | 0.47 | 80 | 33 |
| 9076 | 23 | 0.5 | 60 | 10 |
| 9223.1 | 22.8 | 0.53 | 60 | 7 |
| 9465.1 | 23.3 | 0.5 | 50 | 0 |
| 9868.8 | 29.5 | −0.68 | 10 | 78 |
| 9933.5 | 18.4 | 0.47 | 50 | 3 |
| 10046.3 | 18.1 | 0.61 | 80 | 19 |
| 10518.8 | 20.9 | 0.51 | 70 | 19 |

TABLE 3

| | | | frequency | frequency |
|---|---|---|---|---|
| molecular | migration | discrimination | FSGS | MCD |
| 830.5 | 25.3 | 0.49 | 80 | 31 |
| 865.4 | 35.5 | 0.43 | 80 | 38 |
| 907.4 | 27.5 | 0.41 | 60 | 19 |
| 1005.5 | 35 | 0.45 | 70 | 25 |
| 1008.5 | 34.4 | −0.45 | 30 | 75 |
| 1015.6 | 38.2 | 0.47 | 60 | 13 |
| 1026.5 | 33.2 | −0.4 | 10 | 50 |
| 1041.5 | 51.6 | −0.42 | 20 | 63 |
| 1055.6 | 36.4 | 0.41 | 60 | 19 |
| 1085.6 | 50.8 | −0.42 | 20 | 63 |
| 1088.6 | 37.4 | 0.49 | 80 | 31 |
| 1107.5 | 40.2 | −0.45 | 30 | 75 |
| 1128.5 | 44.3 | 0.41 | 60 | 19 |
| 1138.6 | 22.9 | −0.4 | 10 | 50 |
| 1160.6 | 48.8 | −0.44 | 50 | 94 |
| 1191.6 | 50.5 | −0.49 | 20 | 69 |
| 1199.6 | 31 | −0.63 | 0 | 63 |
| 1207.7 | 36.6 | 0.41 | 60 | 19 |
| 1208.6 | 38.6 | 0.41 | 60 | 19 |
| 1211.6 | 31.3 | 0.43 | 80 | 38 |
| 1224.7 | 33.6 | −0.44 | 50 | 94 |
| 1270.6 | 25.7 | 0.41 | 60 | 19 |
| 1274.6 | 50.7 | −0.44 | 50 | 94 |
| 1282.7 | 38.4 | 0.43 | 80 | 38 |
| 1294.6 | 54.4 | 0.45 | 70 | 25 |
| 1304.8 | 24.6 | 0.44 | 50 | 6 |
| 1305.9 | 33.4 | 0.51 | 70 | 19 |
| 1308.6 | 53.6 | −0.45 | 30 | 75 |
| 1377.7 | 25.4 | 0.45 | 70 | 25 |
| 1390.7 | 41.1 | 0.4 | 90 | 50 |
| 1404.9 | 29.4 | 0.43 | 80 | 38 |
| 1493.7 | 33.7 | 0.51 | 70 | 19 |
| 1518.9 | 42.5 | 0.41 | 60 | 19 |
| 1581 | 37.8 | −0.44 | 50 | 94 |
| 1594.8 | 54.8 | −0.4 | 60 | 100 |
| 1607.7 | 41 | 0.41 | 60 | 19 |
| 1650.7 | 25.4 | −0.46 | 10 | 56 |
| 1695.7 | 54.7 | −0.4 | 10 | 50 |
| 1766.6 | 44.9 | −0.41 | 40 | 81 |
| 1826.9 | 50.8 | −0.59 | 10 | 69 |
| 1880.3 | 57.4 | −0.42 | 20 | 63 |
| 1887.8 | 33.8 | 0.4 | 90 | 50 |
| 1900.7 | 30.4 | −0.61 | 20 | 81 |
| 1925.3 | 52.5 | 0.59 | 90 | 31 |
| 1950.9 | 34.5 | 0.59 | 90 | 31 |
| 1992.9 | 48.5 | 0.44 | 50 | 6 |
| 2005.3 | 39.6 | 0.43 | 80 | 38 |
| 2011.5 | 42 | −0.64 | 30 | 94 |
| 2048.2 | 33.1 | −0.41 | 40 | 81 |
| 2063 | 24.3 | 0.41 | 60 | 19 |
| 2077.3 | 35.8 | −0.59 | 10 | 69 |
| 2121 | 26.9 | 0.43 | 80 | 38 |
| 2163.4 | 27.6 | 0.51 | 70 | 19 |
| 2174.4 | 24.6 | 0.43 | 80 | 38 |
| 2258.9 | 33.6 | 0.75 | 100 | 25 |
| 2412.3 | 42.7 | −0.42 | 20 | 63 |
| 2453.2 | 49.7 | −0.42 | 20 | 63 |
| 2487.9 | 38 | 0.41 | 60 | 19 |
| 2570.5 | 57.1 | −0.42 | 20 | 63 |
| 2679.5 | 35 | −0.44 | 50 | 94 |
| 2690.3 | 24.8 | 0.5 | 50 | 0 |
| 2819.4 | 32.2 | 0.44 | 50 | 6 |
| 2864.7 | 29.1 | −0.49 | 20 | 69 |
| 2883.6 | 28.9 | 0.55 | 80 | 25 |
| 2889.2 | 20.2 | 0.41 | 60 | 19 |
| 2918 | 42.2 | −0.68 | 20 | 88 |
| 2986.9 | 47.3 | −0.41 | 40 | 81 |
| 3209.2 | 34.3 | −0.51 | 30 | 81 |
| 3255.8 | 42.9 | −0.42 | 20 | 63 |
| 3315.1 | 54.1 | −0.4 | 10 | 50 |
| 3402.4 | 33.8 | −0.44 | 50 | 94 |
| 3583.3 | 25.2 | −0.5 | 0 | 50 |
| 4335.8 | 27.1 | 0.44 | 50 | 6 |
| 9182 | 17.1 | −0.42 | 20 | 63 |

TABLE 4

| | | | |
|---|---|---|---|
| molecular | migration | discrimination | frequency [%] |

| weight [Da] | time [min] | factor | FSGS | MGN |
|---|---|---|---|---|
| 819.5 | 35.7 | −0.47 | 20 | 67 |
| 909.4 | 40.3 | −0.4 | 60 | 100 |
| 939.6 | 33.1 | 0.59 | 70 | 11 |
| 978.5 | 23.9 | −0.4 | 10 | 50 |
| 1017.4 | 36.6 | −0.43 | 40 | 83 |
| 1081.7 | 29.6 | 0.49 | 60 | 11 |
| 1201.5 | 51.6 | 0.43 | 60 | 17 |
| 1282.7 | 38.4 | 0.47 | 80 | 33 |
| 1284.8 | 55.1 | −0.4 | 10 | 50 |
| 1305.9 | 33.4 | 0.42 | 70 | 28 |
| 1338.7 | 29.6 | 0.41 | 80 | 39 |
| 1341.8 | 33.1 | −0.43 | 40 | 83 |
| 1359.5 | 47.4 | 0.53 | 70 | 17 |
| 1394.3 | 48.1 | 0.43 | 60 | 17 |
| 1403.8 | 34.4 | 0.43 | 60 | 17 |
| 1423.6 | 22.3 | −0.47 | 20 | 67 |
| 1490.7 | 33.7 | −0.47 | 20 | 67 |
| 1493.5 | 57 | −0.46 | 10 | 56 |
| 1497.9 | 26.7 | −0.48 | 30 | 78 |
| 1504.4 | 30.5 | −0.42 | 30 | 72 |
| 1517.6 | 54.8 | 0.43 | 60 | 17 |
| 1526.4 | 39.4 | −0.42 | 30 | 72 |
| 1529.5 | 32.4 | 0.43 | 60 | 17 |
| 1576.4 | 42.5 | 0.48 | 70 | 22 |
| 1591.7 | 51.2 | 0.44 | 100 | 56 |
| 1595.4 | 31 | −0.48 | 30 | 78 |
| 1692.4 | 30.4 | −0.41 | 20 | 61 |
| 1734.6 | 27.2 | −0.48 | 30 | 78 |
| 1768.9 | 44.7 | 0.63 | 80 | 17 |
| 1790.8 | 38.8 | 0.4 | 90 | 50 |
| 1802.5 | 25.6 | −0.42 | 30 | 72 |
| 1844.2 | 34.6 | 0.4 | 90 | 50 |
| 1883 | 29.1 | 0.44 | 50 | 6 |
| 1885.7 | 57.5 | 0.42 | 70 | 28 |
| 1900.7 | 30.4 | −0.47 | 20 | 67 |
| 1920.5 | 46.1 | 0.49 | 60 | 11 |
| 1925.3 | 52.5 | 0.57 | 90 | 33 |
| 1933.9 | 32.8 | 0.44 | 50 | 6 |
| 1971.5 | 18.9 | −0.47 | 20 | 67 |
| 1986.6 | 35.8 | −0.42 | 30 | 72 |
| 2011.5 | 42 | −0.42 | 30 | 72 |
| 2015.1 | 49.6 | −0.42 | 30 | 72 |
| 2079.7 | 21.8 | −0.51 | 10 | 61 |
| 2121 | 26.9 | 0.47 | 80 | 33 |
| 2129.5 | 35.1 | 0.43 | 60 | 17 |
| 2146.3 | 25.8 | −0.4 | 60 | 100 |
| 2274 | 37 | −0.4 | 10 | 50 |
| 2292.4 | 35.3 | −0.48 | 30 | 78 |
| 2312.5 | 22.9 | −0.63 | 20 | 83 |
| 2338.2 | 40.4 | 0.43 | 60 | 17 |
| 2338.6 | 26 | −0.49 | 40 | 89 |
| 2356.3 | 24 | −0.43 | 40 | 83 |
| 2421 | 28.7 | −0.44 | 50 | 94 |
| 2449.3 | 28.3 | −0.53 | 30 | 83 |
| 2451.7 | 35.5 | −0.43 | 40 | 83 |
| 2453.6 | 32 | −0.53 | 30 | 83 |
| 2469.3 | 32.5 | −0.51 | 10 | 61 |
| 2471.7 | 23.8 | −0.42 | 30 | 72 |
| 2525.5 | 35.6 | 0.68 | 90 | 22 |
| 2566.4 | 22.2 | −0.42 | 30 | 72 |
| 2591.5 | 37.7 | −0.4 | 10 | 50 |
| 2607 | 47.6 | 0.48 | 70 | 22 |
| 2639.6 | 45.2 | −0.46 | 10 | 56 |
| 2665.3 | 39.4 | −0.49 | 40 | 89 |
| 2712.9 | 22.6 | −0.42 | 30 | 72 |
| 2758.5 | 40.9 | 0.42 | 70 | 28 |
| 2912.9 | 57.5 | −0.4 | 10 | 50 |
| 3041.2 | 45 | 0.41 | 80 | 39 |
| 3107.2 | 26.4 | −0.4 | 10 | 50 |
| 3182.9 | 34.3 | 0.44 | 50 | 6 |
| 3313.8 | 31.6 | −0.48 | 30 | 78 |
| 3479.3 | 48.5 | 0.53 | 70 | 17 |
| 4827.1 | 27.3 | −0.43 | 40 | 83 |
| 5829.7 | 20.8 | −0.41 | 20 | 61 |
| 8216.9 | 16.8 | −0.4 | 10 | 50 |
| 8371.2 | 15.8 | −0.41 | 20 | 61 |
| 8466.3 | 18 | −0.51 | 10 | 61 |
| 8518.7 | 15.7 | −0.48 | 30 | 78 |
| 8578.4 | 17 | −0.47 | 20 | 67 |
| 9182 | 17.1 | −0.69 | 20 | 89 |

TABLE 5

| molecular | migration | discrimination | frequency [%] | |
|---|---|---|---|---|
| weight [Da] | time [min] | factor | FSGS | MCD + MGN |
| 939.6 | 33.1 | 0.49 | 70 | 21 |
| 1282.7 | 38.4 | 0.45 | 80 | 35 |
| 1305.9 | 33.4 | 0.46 | 70 | 24 |
| 1359.5 | 47.4 | 0.44 | 70 | 26 |
| 1493.7 | 33.7 | 0.41 | 70 | 29 |
| 1650.7 | 25.4 | −0.4 | 10 | 50 |
| 1734.6 | 27.2 | −0.41 | 30 | 71 |
| 1900.7 | 30.4 | −0.54 | 20 | 74 |
| 1925.3 | 52.5 | 0.58 | 90 | 32 |
| 2011.5 | 42 | −0.52 | 30 | 82 |
| 2121 | 26.9 | 0.45 | 80 | 35 |
| 2258.9 | 33.6 | 0.41 | 100 | 59 |
| 2312.5 | 22.9 | −0.48 | 20 | 68 |
| 2449.3 | 28.3 | −0.41 | 30 | 71 |
| 2525.5 | 35.6 | 0.49 | 90 | 41 |
| 2607 | 47.6 | 0.41 | 70 | 29 |
| 2690.3 | 24.8 | 0.41 | 50 | 9 |
| 2918 | 42.2 | −0.51 | 20 | 71 |
| 9182 | 17.1 | −0.56 | 20 | 76 |

TABLE 6

| molecular | Migration time | discrimination | frequency [%] | |
|---|---|---|---|---|
| weight [Da] | [min] | factor | MCD | control |
| 836.5 | 35 | 0.59 | 75 | 16 |
| 862.4 | 48.7 | 0.44 | 56 | 12 |
| 866.4 | 37.9 | 0.41 | 56 | 16 |
| 866.5 | 23.1 | 0.55 | 56 | 2 |
| 870.4 | 33.9 | 0.43 | 50 | 7 |
| 876.4 | 48.9 | −0.61 | 38 | 98 |
| 881.5 | 25.7 | 0.46 | 75 | 29 |
| 882.6 | 36.5 | −0.64 | 31 | 95 |
| 888.6 | 29.9 | −0.47 | 6 | 53 |
| 903.4 | 46.8 | −0.49 | 6 | 55 |
| 914.5 | 34.3 | 0.47 | 50 | 3 |
| 925.4 | 50.7 | 0.61 | 75 | 14 |
| 943.5 | 30 | 0.47 | 75 | 28 |
| 950.5 | 36 | 0.51 | 56 | 5 |
| 956.4 | 49.5 | −0.48 | 13 | 60 |
| 958.5 | 32.5 | 0.46 | 69 | 22 |
| 974.5 | 37.9 | 0.41 | 50 | 9 |
| 988.5 | 33.9 | 0.43 | 50 | 7 |
| 990.6 | 32 | 0.44 | 63 | 19 |
| 991.4 | 37.7 | 0.42 | 56 | 14 |
| 1000.5 | 34 | −0.51 | 44 | 95 |
| 1006.4 | 35.7 | −0.4 | 38 | 78 |
| 1010.6 | 50.8 | −0.45 | 19 | 64 |
| 1010.6 | 30.6 | −0.52 | 31 | 83 |
| 1033.6 | 39.1 | 0.41 | 50 | 9 |
| 1034.5 | 31.4 | 0.47 | 50 | 3 |
| 1046.6 | 38.6 | −0.59 | 38 | 97 |
| 1047.6 | 30.4 | −0.47 | 38 | 84 |
| 1085.6 | 50.8 | 0.49 | 63 | 14 |
| 1102.6 | 32.9 | 0.44 | 56 | 12 |
| 1104.6 | 43.3 | −0.45 | 6 | 52 |
| 1108.6 | 29.8 | 0.42 | 81 | 40 |
| 1110.4 | 46.9 | −0.46 | 25 | 71 |
| 1122.5 | 50.2 | −0.41 | 25 | 66 |
| 1135.6 | 42.7 | −0.53 | 13 | 66 |
| 1138.6 | 39.3 | 0.7 | 88 | 17 |
| 1138.6 | 22.9 | 0.48 | 50 | 2 |
| 1139.6 | 32.2 | 0.49 | 56 | 7 |
| 1141.6 | 38 | −0.61 | 6 | 67 |
| 1159.6 | 39 | −0.56 | 25 | 81 |
| 1171.6 | 32.8 | 0.8 | 81 | 2 |
| 1182.6 | 47.2 | −0.41 | 25 | 66 |
| 1191.8 | 18.3 | 0.46 | 56 | 10 |
| 1199.6 | 31 | 0.61 | 63 | 2 |
| 1203.7 | 24.7 | −0.51 | 6 | 57 |
| 1219.6 | 37.3 | 0.48 | 69 | 21 |
| 1223.5 | 51.6 | −0.65 | 25 | 90 |
| 1233.7 | 49.6 | 0.45 | 50 | 5 |
| 1237.7 | 41.6 | −0.42 | 25 | 67 |
| 1246.7 | 30.5 | −0.41 | 25 | 66 |
| 1256.6 | 53.4 | 0.49 | 63 | 14 |
| 1264.7 | 26.7 | 0.66 | 75 | 9 |
| 1268.6 | 53.7 | −0.45 | 6 | 52 |
| 1269.7 | 39.8 | 0.44 | 63 | 19 |
| 1270.5 | 52.5 | −0.49 | 6 | 55 |
| 1274.6 | 50.7 | 0.49 | 94 | 45 |
| 1280.6 | 51.9 | −0.5 | 13 | 62 |
| 1292.5 | 53 | −0.49 | 38 | 86 |
| 1296.6 | 53.8 | 0.53 | 56 | 3 |
| 1302.7 | 31.8 | 0.55 | 88 | 33 |
| 1310.7 | 36.8 | 0.41 | 56 | 16 |
| 1311.8 | 31.5 | 0.44 | 63 | 19 |
| 1324.2 | 40.5 | 0.6 | 69 | 9 |
| 1324.5 | 54.3 | 0.45 | 63 | 17 |
| 1325.5 | 35.2 | 0.55 | 81 | 26 |
| 1333.8 | 38.8 | 0.52 | 69 | 17 |
| 1338.7 | 47.2 | 0.86 | 88 | 2 |
| 1338.7 | 29.6 | 0.52 | 63 | 10 |
| 1350.7 | 50.3 | −0.44 | 6 | 50 |
| 1354.8 | 45.6 | 0.62 | 100 | 38 |
| 1365 | 22.3 | 0.49 | 63 | 14 |
| 1371.8 | 19.3 | 0.49 | 75 | 26 |
| 1389.8 | 19.5 | 0.41 | 75 | 34 |
| 1401.8 | 46.2 | −0.5 | 13 | 62 |
| 1414.6 | 38.1 | 0.57 | 81 | 24 |
| 1415.7 | 33.3 | 0.51 | 56 | 5 |
| 1424.9 | 35.4 | −0.52 | 19 | 71 |
| 1442.8 | 33.3 | 0.61 | 81 | 21 |
| 1444.6 | 37.8 | 0.46 | 75 | 29 |
| 1448.8 | 30.3 | 0.42 | 75 | 33 |
| 1472.1 | 31.2 | 0.42 | 56 | 14 |
| 1474.9 | 16.9 | 0.63 | 75 | 12 |
| 1482 | 30.4 | 0.47 | 75 | 28 |
| 1484 | 30.4 | 0.5 | 81 | 31 |
| 1486.5 | 30.6 | 0.61 | 81 | 21 |
| 1499.9 | 30.6 | 0.53 | 88 | 34 |
| 1502.8 | 28.8 | 0.5 | 81 | 31 |
| 1502.9 | 16.8 | 0.55 | 56 | 2 |
| 1508.9 | 16.8 | 0.54 | 63 | 9 |
| 1511.7 | 38.4 | 0.7 | 94 | 24 |
| 1535 | 28.3 | 0.57 | 69 | 12 |
| 1548.3 | 31.1 | 0.44 | 88 | 43 |
| 1556.8 | 33.7 | 0.52 | 81 | 29 |
| 1561.9 | 28.1 | 0.46 | 88 | 41 |
| 1567.6 | 53.9 | −0.55 | 0 | 55 |
| 1573.8 | 40.4 | 0.43 | 88 | 45 |
| 1574.3 | 53.4 | −0.41 | 13 | 53 |
| 1588.4 | 47.9 | −0.6 | 13 | 72 |
| 1591.6 | 32.6 | 0.49 | 56 | 7 |
| 1596.9 | 34 | 0.53 | 75 | 22 |
| 1604.3 | 21.6 | 0.55 | 69 | 14 |
| 1611.7 | 53.2 | −0.41 | 44 | 84 |
| 1612.8 | 36.8 | 0.55 | 88 | 33 |
| 1622 | 19.2 | 0.42 | 81 | 40 |
| 1629.6 | 49.6 | 0.47 | 50 | 3 |
| 1635.2 | 27.8 | 0.41 | 75 | 34 |
| 1644 | 18.8 | 0.41 | 50 | 9 |
| 1658.4 | 39 | 0.44 | 88 | 43 |
| 1669.9 | 33.4 | 0.48 | 69 | 21 |
| 1671.3 | 42.6 | 0.42 | 56 | 14 |
| 1676 | 25.3 | 0.44 | 56 | 12 |
| 1681.6 | 40 | 0.56 | 88 | 31 |
| 1686.8 | 38.2 | 0.63 | 88 | 24 |
| 1692.4 | 30.4 | 0.41 | 56 | 16 |
| 1699.1 | 41.9 | 0.63 | 88 | 24 |
| 1718.5 | 22.6 | 0.48 | 56 | 9 |
| 1746.2 | 46.2 | −0.53 | 31 | 84 |
| 1747.7 | 50.8 | −0.51 | 6 | 57 |
| 1751.4 | 40.8 | 0.43 | 81 | 38 |
| 1752.9 | 39.9 | 0.46 | 69 | 22 |
| 1766.6 | 44.9 | 0.52 | 81 | 29 |
| 1776.1 | 43.6 | −0.45 | 38 | 83 |
| 1777.6 | 28.6 | 0.58 | 75 | 17 |
| 1804.7 | 34 | 0.45 | 100 | 55 |
| 1811.3 | 31.3 | 0.5 | 88 | 38 |
| 1813.4 | 54.7 | −0.53 | 0 | 53 |
| 1815.2 | 27.7 | 0.41 | 56 | 16 |
| 1820.1 | 31.8 | 0.44 | 88 | 43 |
| 1821.2 | 18.2 | 0.45 | 50 | 5 |
| 1822.9 | 40.7 | −0.52 | 31 | 83 |
| 1824.3 | 37 | −0.42 | 38 | 79 |
| 1831.9 | 41.5 | 0.54 | 63 | 9 |
| 1847.8 | 57 | −0.62 | 31 | 93 |
| 1851.2 | 31.6 | 0.43 | 81 | 38 |
| 1853 | 31.2 | 0.4 | 63 | 22 |
| 1854.9 | 53.6 | −0.44 | 6 | 50 |
| 1856.8 | 56.3 | −0.44 | 25 | 69 |
| 1864.6 | 28.6 | 0.64 | 81 | 17 |
| 1867 | 31.8 | 0.56 | 88 | 31 |
| 1889.8 | 46.4 | −0.53 | 44 | 97 |
| 1894.9 | 22 | 0.56 | 75 | 19 |
| 1896.8 | 53.3 | −0.43 | 13 | 55 |
| 1909.7 | 47.9 | 0.49 | 63 | 14 |
| 1913.4 | 30.1 | 0.46 | 56 | 10 |
| 1916.8 | 44.7 | −0.59 | 6 | 66 |
| 1934.2 | 16.1 | 0.48 | 50 | 2 |
| 1944.2 | 47 | −0.61 | 25 | 86 |
| 1951.1 | 53 | −0.45 | 19 | 64 |
| 1955.3 | 48.4 | 0.44 | 63 | 19 |
| 1966.3 | 25.1 | 0.65 | 75 | 10 |
| 1973.7 | 57.1 | −0.46 | 13 | 59 |
| 1982.9 | 32.2 | 0.57 | 81 | 24 |
| 1989.3 | 43.7 | 0.66 | 81 | 16 |
| 1990.8 | 47.3 | −0.7 | 13 | 83 |
| 2011.5 | 42 | 0.64 | 94 | 29 |
| 2017.6 | 33.2 | 0.45 | 81 | 36 |
| 2030.4 | 31.7 | −0.44 | 38 | 81 |
| 2030.8 | 46.5 | −0.61 | 25 | 86 |
| 2047 | 45.4 | −0.42 | 56 | 98 |
| 2050.8 | 38.2 | 0.47 | 81 | 34 |
| 2092.5 | 41.3 | 0.66 | 81 | 16 |
| 2098.3 | 52 | 0.5 | 69 | 19 |
| 2099.2 | 36.9 | 0.49 | 75 | 26 |
| 2103.6 | 26.7 | 0.44 | 88 | 43 |
| 2106.1 | 46.1 | 0.41 | 56 | 16 |
| 2117.1 | 57.1 | −0.4 | 38 | 78 |
| 2129.5 | 35.1 | −0.47 | 50 | 97 |
| 2130.3 | 18.4 | 0.42 | 56 | 14 |
| 2139.3 | 36.9 | 0.41 | 56 | 16 |
| 2146.3 | 25.8 | 0.59 | 63 | 3 |
| 2151.6 | 42.6 | 0.44 | 56 | 12 |
| 2157.2 | 24.4 | 0.54 | 63 | 9 |
| 2182.5 | 27.6 | 0.51 | 75 | 24 |
| 2189.1 | 40.9 | −0.48 | 19 | 67 |
| 2207.2 | 41.9 | 0.48 | 69 | 21 |
| 2210.7 | 25.7 | 0.59 | 81 | 22 |
| 2217.7 | 41.9 | 0.53 | 88 | 34 |
| 2223.5 | 22.6 | 0.54 | 63 | 9 |
| 2228.1 | 25.9 | 0.48 | 88 | 40 |
| 2238.4 | 46.3 | −0.41 | 44 | 84 |
| 2281.7 | 45.6 | −0.5 | 31 | 81 |
| 2426.5 | 38.5 | 0.56 | 88 | 31 |
| 2432.2 | 38.3 | 0.55 | 69 | 14 |
| 2464 | 47.2 | −0.5 | 13 | 62 |
| 2465 | 22.8 | 0.51 | 56 | 5 |
| 2522.9 | 24.4 | 0.42 | 63 | 21 |
| 2529.2 | 41.4 | −0.42 | 19 | 60 |
| 2535 | 37.7 | 0.42 | 81 | 40 |
| 2540.5 | 25.5 | 0.58 | 69 | 10 |
| 2548.2 | 35.1 | −0.45 | 38 | 83 |
| 2566.4 | 22.2 | 0.49 | 56 | 7 |
| 2593.4 | 25 | 0.41 | 56 | 16 |
| 2621.4 | 25.8 | 0.5 | 63 | 12 |
| 2644.1 | 32.5 | −0.43 | 50 | 93 |
| 2698.4 | 32.1 | −0.48 | 31 | 79 |
| 2713.2 | 41.3 | −0.43 | 19 | 62 |
| 2752.8 | 25.3 | 0.49 | 75 | 26 |
| 2790.3 | 26.8 | 0.41 | 56 | 16 |
| 2793.7 | 36.3 | 0.66 | 81 | 16 |
| 2809.1 | 37.2 | −0.46 | 31 | 78 |
| 2921.4 | 30.4 | 0.43 | 69 | 26 |
| 2933.8 | 39.4 | −0.47 | 6 | 53 |
| 2973.7 | 34.9 | −0.5 | 38 | 88 |
| 3007.5 | 30.5 | −0.48 | 19 | 67 |
| 3017.7 | 46.8 | −0.42 | 19 | 60 |
| 3139.4 | 43.7 | −0.4 | 38 | 78 |
| 3179.2 | 44.3 | 0.41 | 75 | 34 |
| 3262 | 31.5 | −0.49 | 38 | 86 |
| 3281 | 36.8 | −0.48 | 50 | 98 |
| 3282 | 49.4 | −0.45 | 50 | 95 |
| 3290.9 | 36.9 | −0.56 | 38 | 93 |
| 3295.8 | 38.4 | −0.48 | 50 | 98 |
| 3333.4 | 23.3 | −0.5 | 38 | 88 |
| 3334.6 | 41.7 | −0.42 | 44 | 86 |
| 3343.8 | 43.8 | −0.41 | 50 | 91 |
| 3433.3 | 44.5 | −0.42 | 56 | 98 |
| 3530.9 | 36.8 | −0.5 | 31 | 81 |
| 3589.5 | 39.1 | −0.58 | 31 | 90 |
| 3631.2 | 33.1 | −0.52 | 19 | 71 |
| 3686.1 | 32.6 | −0.7 | 13 | 83 |
| 3697.4 | 38.8 | −0.41 | 13 | 53 |
| 3701.8 | 43.4 | −0.54 | 19 | 72 |
| 3707 | 31.9 | −0.63 | 13 | 76 |
| 3723.3 | 32.5 | −0.47 | 50 | 97 |
| 3760.8 | 25.9 | −0.5 | 19 | 69 |
| 3816.7 | 32.2 | −0.47 | 19 | 66 |
| 4154.2 | 23.7 | 0.42 | 56 | 14 |
| 4170.6 | 46.1 | −0.45 | 6 | 52 |
| 4241.2 | 24.4 | 0.67 | 81 | 14 |
| 4283.1 | 24.3 | 0.54 | 63 | 9 |
| 4707.5 | 20.5 | 0.42 | 56 | 14 |
| 4713.7 | 26.9 | 0.45 | 50 | 5 |
| 4748.5 | 25.4 | −0.57 | 38 | 95 |
| 4772.1 | 28.9 | −0.45 | 6 | 52 |
| 5213.8 | 36.8 | −0.44 | 6 | 50 |
| 7409.9 | 26.2 | 0.44 | 56 | 12 |
| 7556.6 | 26.2 | 0.53 | 69 | 16 |
| 8054.8 | 16.7 | 0.5 | 69 | 19 |
| 8765.9 | 17.6 | 0.48 | 81 | 33 |
| 9076 | 23 | 0.58 | 69 | 10 |
| 9182 | 17.1 | 0.54 | 63 | 9 |
| 9223.1 | 22.8 | 0.56 | 63 | 7 |
| 9868.8 | 29.5 | −0.53 | 25 | 78 |
| 10046.3 | 18.1 | 0.62 | 81 | 19 |
| 10390.1 | 20.2 | 0.5 | 63 | 12 |
| 10518.8 | 20.9 | 0.62 | 81 | 19 |

TABLE 7

| | | | |
|---|---|---|---|
| molecular | migration | discrimination | frequency [%] |

| weight [Da] | time [min] | factor | MCD | MGN |
|---|---|---|---|---|
| 814.5 | 28.8 | −0.46 | 38 | 83 |
| 819.5 | 35.7 | −0.48 | 19 | 67 |
| 856.5 | 28.9 | 0.42 | 75 | 33 |
| 863.4 | 28.8 | −0.54 | 13 | 67 |
| 864.5 | 37.3 | −0.48 | 19 | 67 |
| 879.6 | 26.9 | 0.72 | 94 | 22 |
| 882.6 | 36.5 | −0.41 | 31 | 72 |
| 909.4 | 40.3 | −0.56 | 44 | 100 |
| 928.4 | 49.4 | 0.53 | 81 | 28 |
| 934.5 | 33.9 | −0.41 | 31 | 72 |
| 935.6 | 36.6 | 0.46 | 63 | 17 |
| 946.5 | 46.8 | 0.47 | 69 | 22 |
| 952.5 | 32 | −0.46 | 38 | 83 |
| 1005.5 | 35 | −0.42 | 25 | 67 |
| 1008.5 | 34.4 | 0.53 | 75 | 22 |
| 1015.6 | 38.2 | −0.71 | 13 | 83 |
| 1017.4 | 36.6 | −0.52 | 31 | 83 |
| 1022.5 | 39.1 | −0.52 | 31 | 83 |
| 1028.6 | 37.8 | 0.47 | 69 | 22 |
| 1073.6 | 34.7 | −0.56 | 44 | 100 |
| 1085.6 | 50.8 | 0.51 | 63 | 11 |
| 1108.5 | 50.1 | 0.45 | 56 | 11 |
| 1113.6 | 33.7 | −0.48 | 19 | 67 |
| 1138.6 | 22.9 | 0.44 | 50 | 6 |
| 1147.6 | 49.7 | 0.46 | 63 | 17 |
| 1152.5 | 40.7 | −0.65 | 19 | 83 |
| 1208.6 | 38.6 | −0.42 | 19 | 61 |
| 1211.6 | 31.3 | −0.46 | 38 | 83 |
| 1213.6 | 50 | −0.43 | 13 | 56 |
| 1224.7 | 33.6 | 0.6 | 94 | 33 |
| 1225.7 | 41.3 | −0.42 | 25 | 67 |
| 1270.6 | 25.7 | −0.48 | 19 | 67 |
| 1277.6 | 50 | 0.46 | 63 | 17 |
| 1279.7 | 38.3 | −0.63 | 31 | 94 |
| 1283.9 | 28.9 | −0.48 | 19 | 67 |
| 1301.7 | 34 | −0.43 | 13 | 56 |
| 1319.9 | 34.8 | −0.46 | 38 | 83 |
| 1329.8 | 37.5 | −0.47 | 25 | 72 |
| 1337.6 | 52 | 0.47 | 69 | 22 |
| 1341.8 | 33.1 | −0.58 | 25 | 83 |
| 1350.8 | 26.8 | −0.42 | 19 | 61 |
| 1365 | 22.3 | 0.4 | 63 | 22 |
| 1381.1 | 32.3 | −0.63 | 31 | 94 |
| 1398.9 | 30.5 | −0.44 | 50 | 94 |
| 1404.9 | 29.4 | −0.63 | 38 | 100 |
| 1423.6 | 22.3 | −0.48 | 19 | 67 |
| 1426.8 | 38.7 | −0.42 | 25 | 67 |
| 1433 | 33.7 | −0.53 | 19 | 72 |
| 1465.9 | 28.8 | −0.45 | 44 | 89 |
| 1482.8 | 36.3 | −0.46 | 38 | 83 |
| 1487.7 | 41.4 | 0.54 | 88 | 33 |
| 1490.7 | 33.7 | −0.48 | 19 | 67 |
| 1512.8 | 35.9 | 0.41 | 69 | 28 |
| 1527.9 | 34.7 | −0.44 | 50 | 94 |
| 1543.8 | 34.9 | −0.52 | 31 | 83 |
| 1558.1 | 23.4 | −0.42 | 19 | 61 |
| 1560.5 | 39.5 | −0.46 | 38 | 83 |
| 1569.8 | 48.3 | 0.5 | 50 | 0 |
| 1574.8 | 33.9 | −0.58 | 31 | 89 |
| 1593.8 | 36.7 | 0.41 | 69 | 28 |
| 1595.4 | 31 | −0.4 | 38 | 78 |
| 1602.8 | 58.1 | 0.51 | 63 | 11 |
| 1605.9 | 23.7 | −0.65 | 13 | 78 |
| 1607.7 | 41 | −0.42 | 19 | 61 |
| 1612.8 | 26.3 | −0.43 | 13 | 56 |
| 1623.3 | 41.4 | −0.47 | 31 | 78 |
| 1671.3 | 42.6 | 0.45 | 56 | 11 |
| 1726 | 36.3 | −0.51 | 44 | 94 |
| 1729.2 | 26 | −0.45 | 44 | 89 |
| 1744.1 | 34.3 | −0.63 | 38 | 100 |
| 1768.9 | 44.7 | 0.52 | 69 | 17 |
| 1774.6 | 36.5 | −0.47 | 31 | 78 |
| 1786.9 | 35.9 | −0.41 | 31 | 72 |
| 1799 | 28.8 | −0.47 | 25 | 72 |
| 1802.5 | 25.6 | −0.53 | 19 | 72 |
| 1826.9 | 50.8 | 0.52 | 69 | 17 |
| 1839.1 | 35.5 | −0.44 | 50 | 94 |
| 1857.1 | 39 | −0.44 | 50 | 94 |
| 1859.4 | 22.8 | −0.42 | 25 | 67 |
| 1863.8 | 57.5 | 0.42 | 81 | 39 |
| 1876.2 | 40.1 | −0.51 | 38 | 89 |
| 1878.7 | 49.9 | 0.47 | 75 | 28 |
| 1880.3 | 57.4 | 0.51 | 63 | 11 |
| 1883 | 29.1 | 0.44 | 50 | 6 |
| 1885.7 | 57.5 | 0.47 | 75 | 28 |
| 1887.8 | 33.8 | −0.5 | 50 | 100 |
| 1898.7 | 26.5 | −0.52 | 31 | 83 |
| 1924.2 | 32.9 | −0.64 | 25 | 89 |
| 1933.9 | 32.8 | 0.57 | 63 | 6 |
| 1936.7 | 32.8 | −0.44 | 56 | 100 |
| 1949.1 | 38.5 | 0.43 | 88 | 44 |
| 1950.9 | 34.5 | −0.58 | 31 | 89 |
| 1971.5 | 18.9 | −0.48 | 19 | 67 |
| 1977.4 | 42.9 | 0.4 | 63 | 22 |
| 1988.9 | 28.8 | −0.4 | 38 | 78 |
| 2005.3 | 39.6 | −0.46 | 38 | 83 |
| 2011.3 | 29 | −0.43 | 13 | 56 |
| 2033.5 | 27.5 | −0.53 | 25 | 78 |
| 2035.6 | 30.9 | −0.54 | 13 | 67 |
| 2065.3 | 20.9 | −0.47 | 25 | 72 |
| 2077.3 | 35.8 | 0.41 | 69 | 28 |
| 2109.3 | 27.9 | −0.51 | 44 | 94 |
| 2140.1 | 26.8 | −0.51 | 38 | 89 |
| 2152.7 | 29.5 | −0.51 | 44 | 94 |
| 2160.4 | 27.9 | −0.49 | 6 | 56 |
| 2163.4 | 27.6 | −0.48 | 19 | 67 |
| 2167.3 | 27.8 | −0.41 | 31 | 72 |
| 2174.4 | 24.6 | −0.51 | 38 | 89 |
| 2178.5 | 21.4 | −0.4 | 38 | 78 |
| 2189.1 | 40.9 | −0.48 | 19 | 67 |
| 2258.9 | 33.6 | −0.64 | 25 | 89 |
| 2274 | 37 | −0.44 | 6 | 50 |
| 2288.8 | 41.4 | −0.65 | 13 | 78 |
| 2291.1 | 21.9 | −0.47 | 25 | 72 |
| 2292.4 | 35.3 | −0.4 | 38 | 78 |
| 2308.9 | 26.2 | −0.46 | 38 | 83 |
| 2332.4 | 35.4 | −0.54 | 13 | 67 |
| 2341.2 | 26.3 | −0.49 | 6 | 56 |
| 2356.3 | 24 | −0.46 | 38 | 83 |
| 2367.7 | 43.2 | 0.58 | 75 | 17 |
| 2380 | 39.6 | −0.51 | 44 | 94 |
| 2391.2 | 24.3 | −0.44 | 50 | 94 |
| 2423.1 | 27.4 | −0.45 | 44 | 89 |
| 2434.4 | 34.7 | −0.44 | 6 | 50 |
| 2446.2 | 24.7 | −0.42 | 19 | 61 |
| 2451.7 | 35.5 | −0.46 | 38 | 83 |
| 2453.6 | 32 | −0.52 | 31 | 83 |
| 2453.8 | 20.4 | −0.49 | 6 | 56 |
| 2455.6 | 27.7 | −0.41 | 31 | 72 |
| 2461.1 | 40.5 | −0.47 | 25 | 72 |
| 2469.3 | 32.5 | −0.42 | 19 | 61 |
| 2471.7 | 23.8 | −0.41 | 31 | 72 |
| 2475.5 | 22.3 | −0.42 | 19 | 61 |
| 2480.2 | 47.2 | 0.4 | 63 | 22 |
| 2483.8 | 19.6 | −0.47 | 25 | 72 |
| 2493.6 | 24.6 | −0.5 | 50 | 100 |
| 2500.3 | 30.4 | 0.53 | 75 | 22 |
| 2518.7 | 38.9 | −0.46 | 38 | 83 |
| 2521.3 | 48.3 | −0.49 | 13 | 61 |
| 2525.5 | 35.6 | 0.4 | 63 | 22 |
| 2527.3 | 40.8 | −0.53 | 19 | 72 |
| 2553.7 | 24.7 | −0.42 | 19 | 61 |
| 2573.7 | 16.3 | −0.45 | 44 | 89 |
| 2579.5 | 15.2 | −0.48 | 19 | 67 |
| 2608.6 | 57.7 | 0.45 | 56 | 11 |
| 2614.1 | 22.5 | −0.47 | 31 | 78 |
| 2619.6 | 38.3 | −0.42 | 19 | 61 |
| 2642.4 | 40.9 | 0.46 | 63 | 17 |
| 2660.8 | 27.1 | −0.47 | 31 | 78 |
| 2665.3 | 39.4 | −0.58 | 31 | 89 |
| 2666 | 23 | −0.43 | 13 | 56 |
| 2677.6 | 23.6 | −0.51 | 44 | 94 |
| 2701 | 34.8 | 0.4 | 63 | 22 |
| 2784.3 | 45.2 | −0.59 | 19 | 78 |
| 2825.4 | 36.5 | 0.49 | 88 | 39 |
| 2830.9 | 33.2 | −0.57 | 38 | 94 |
| 2864.7 | 29.1 | 0.52 | 69 | 17 |
| 2889.2 | 20.2 | −0.42 | 19 | 61 |
| 2902.9 | 42.1 | −0.42 | 25 | 67 |
| 2912.9 | 57.5 | −0.44 | 6 | 50 |
| 2921.4 | 30.4 | 0.52 | 69 | 17 |
| 2940.5 | 40.4 | −0.4 | 38 | 78 |
| 3041.2 | 45 | 0.42 | 81 | 39 |
| 3044.8 | 48.6 | 0.4 | 63 | 22 |
| 3082.3 | 43.1 | 0.42 | 75 | 33 |
| 3169 | 37.5 | 0.42 | 75 | 33 |
| 3205.8 | 28.3 | 0.53 | 75 | 22 |
| 3209.2 | 34.3 | 0.48 | 81 | 33 |
| 3255.8 | 42.9 | 0.4 | 63 | 22 |
| 3256.3 | 23.1 | −0.48 | 19 | 67 |
| 3303.2 | 38.6 | 0.44 | 50 | 6 |
| 3308.6 | 21.3 | −0.47 | 31 | 78 |
| 3313.8 | 31.6 | −0.53 | 25 | 78 |
| 3325.5 | 43.5 | 0.44 | 50 | 6 |
| 3336.8 | 53.8 | 0.51 | 56 | 6 |
| 3405.7 | 37.8 | 0.46 | 63 | 17 |
| 3422.5 | 38.7 | 0.45 | 56 | 11 |
| 3479.3 | 48.5 | 0.58 | 75 | 17 |
| 3578.2 | 32.5 | −0.53 | 19 | 72 |
| 3881.9 | 26.2 | −0.42 | 19 | 61 |
| 3969.6 | 31.3 | 0.45 | 56 | 11 |
| 4183.7 | 26.6 | −0.47 | 31 | 78 |
| 4290.8 | 41.1 | 0.4 | 63 | 22 |
| 4527.7 | 26 | −0.53 | 19 | 72 |
| 4565.8 | 25.1 | −0.44 | 6 | 50 |
| 4719.5 | 39.3 | −0.44 | 6 | 50 |
| 4827.1 | 27.3 | −0.58 | 25 | 83 |
| 5112.9 | 33.1 | −0.5 | 0 | 50 |
| 5829.7 | 20.8 | −0.49 | 13 | 61 |
| 6106.5 | 27 | −0.56 | 0 | 56 |
| 7885.4 | 20.9 | −0.49 | 13 | 61 |
| 8341.2 | 16.6 | −0.57 | 38 | 94 |
| 8371.2 | 15.8 | −0.49 | 13 | 61 |
| 8466.3 | 18 | −0.42 | 19 | 61 |
| 8518.7 | 15.7 | −0.53 | 25 | 78 |
| 8578.4 | 17 | −0.48 | 19 | 67 |
| 9335.5 | 17.5 | −0.41 | 31 | 72 |
| 9465.1 | 23.3 | −0.49 | 13 | 61 |
| 9944.2 | 16.7 | −0.48 | 19 | 67 |
| 10949.7 | 26.3 | −0.56 | 0 | 56 |

TABLE 8

| molecular | migration | discrimination | frequency [%] | |
|---|---|---|---|---|
| weight [Da] | time [min] | factor | MCD | FSGS + MGN |
| 863.4 | 28.8 | −0.45 | 13 | 57 |
| 879.6 | 26.9 | 0.58 | 94 | 36 |
| 909.4 | 40.3 | −0.42 | 44 | 86 |
| 928.4 | 49.4 | 0.46 | 81 | 36 |
| 935.6 | 36.6 | 0.41 | 63 | 21 |
| 946.5 | 46.8 | 0.44 | 69 | 25 |
| 952.5 | 32 | −0.41 | 38 | 79 |
| 1005.5 | 35 | −0.43 | 25 | 68 |
| 1008.5 | 34.4 | 0.5 | 75 | 25 |
| 1015.6 | 38.2 | −0.63 | 13 | 75 |
| 1022.5 | 39.1 | −0.44 | 31 | 75 |
| 1028.6 | 37.8 | 0.4 | 69 | 29 |
| 1073.6 | 34.7 | −0.49 | 44 | 93 |
| 1085.6 | 50.8 | 0.48 | 63 | 14 |
| 1138.6 | 22.9 | 0.43 | 50 | 7 |
| 1147.6 | 49.7 | 0.41 | 63 | 21 |
| 1152.5 | 40.7 | −0.53 | 19 | 71 |
| 1199.6 | 31 | 0.41 | 63 | 21 |
| 1208.6 | 38.6 | −0.42 | 19 | 61 |
| 1211.6 | 31.3 | −0.45 | 38 | 82 |
| 1224.7 | 33.6 | 0.54 | 94 | 39 |
| 1270.6 | 25.7 | −0.46 | 19 | 64 |
| 1279.7 | 38.3 | −0.51 | 31 | 82 |
| 1341.8 | 33.1 | −0.43 | 25 | 68 |
| 1381.1 | 32.3 | −0.54 | 31 | 86 |
| 1404.9 | 29.4 | −0.55 | 38 | 93 |
| 1433 | 33.7 | −0.46 | 19 | 64 |
| 1487.7 | 41.4 | 0.48 | 88 | 39 |
| 1543.8 | 34.9 | −0.44 | 31 | 75 |
| 1560.5 | 39.5 | −0.41 | 38 | 79 |
| 1569.8 | 48.3 | 0.43 | 50 | 7 |
| 1574.8 | 33.9 | −0.47 | 31 | 79 |
| 1602.8 | 58.1 | 0.41 | 63 | 21 |
| 1605.9 | 23.7 | −0.52 | 13 | 64 |
| 1607.7 | 41 | −0.42 | 19 | 61 |
| 1623.3 | 41.4 | −0.44 | 31 | 75 |
| 1726 | 36.3 | −0.42 | 44 | 86 |
| 1729.2 | 26 | −0.42 | 44 | 86 |
| 1744.1 | 34.3 | −0.52 | 38 | 89 |
| 1786.9 | 35.9 | −0.4 | 31 | 71 |
| 1826.9 | 50.8 | 0.54 | 69 | 14 |
| 1876.2 | 40.1 | −0.41 | 38 | 79 |
| 1880.3 | 57.4 | 0.48 | 63 | 14 |
| 1887.8 | 33.8 | −0.46 | 50 | 96 |
| 1898.7 | 26.5 | −0.44 | 31 | 75 |
| 1924.2 | 32.9 | −0.54 | 25 | 79 |
| 1933.9 | 32.8 | 0.41 | 63 | 21 |
| 1950.9 | 34.5 | −0.58 | 31 | 89 |
| 2005.3 | 39.6 | −0.45 | 38 | 82 |
| 2033.5 | 27.5 | −0.46 | 25 | 71 |
| 2035.6 | 30.9 | −0.48 | 13 | 61 |
| 2065.3 | 20.9 | −0.43 | 25 | 68 |
| 2077.3 | 35.8 | 0.47 | 69 | 21 |
| 2140.1 | 26.8 | −0.41 | 38 | 79 |
| 2152.7 | 29.5 | −0.42 | 44 | 86 |
| 2163.4 | 27.6 | −0.49 | 19 | 68 |
| 2174.4 | 24.6 | −0.48 | 38 | 86 |
| 2189.1 | 40.9 | −0.42 | 19 | 61 |
| 2258.9 | 33.6 | −0.68 | 25 | 93 |
| 2288.8 | 41.4 | −0.55 | 13 | 68 |
| 2332.4 | 35.4 | −0.45 | 13 | 57 |
| 2367.7 | 43.2 | 0.5 | 75 | 25 |
| 2493.6 | 24.6 | −0.43 | 50 | 93 |
| 2500.3 | 30.4 | 0.43 | 75 | 32 |
| 2527.3 | 40.8 | −0.42 | 19 | 61 |
| 2614.1 | 22.5 | −0.44 | 31 | 75 |
| 2660.8 | 27.1 | −0.44 | 31 | 75 |
| 2665.3 | 39.4 | −0.4 | 31 | 71 |
| 2784.3 | 45.2 | −0.46 | 19 | 64 |
| 2825.4 | 36.5 | 0.45 | 88 | 43 |
| 2830.9 | 33.2 | −0.48 | 38 | 86 |
| 2864.7 | 29.1 | 0.51 | 69 | 18 |
| 2883.6 | 28.9 | −0.43 | 25 | 68 |
| 2889.2 | 20.2 | −0.42 | 19 | 61 |
| 2918 | 42.2 | 0.45 | 88 | 43 |
| 2921.4 | 30.4 | 0.47 | 69 | 21 |
| 3205.8 | 28.3 | 0.43 | 75 | 32 |
| 3209.2 | 34.3 | 0.49 | 81 | 32 |
| 3255.8 | 42.9 | 0.41 | 63 | 21 |
| 3308.6 | 21.3 | −0.4 | 31 | 71 |
| 3402.4 | 33.8 | 0.4 | 94 | 54 |
| 3578.2 | 32.5 | −0.46 | 19 | 64 |
| 3583.3 | 25.2 | 0.43 | 50 | 7 |
| 4527.7 | 26 | −0.46 | 19 | 64 |
| 4827.1 | 27.3 | −0.43 | 25 | 68 |
| 7885.4 | 20.9 | −0.45 | 13 | 57 |
| 8341.2 | 16.6 | −0.45 | 38 | 82 |
| 9465.1 | 23.3 | −0.45 | 13 | 57 |

TABLE 9

| | | | |
|---|---|---|---|
| molecular | Migration | discrimination | frequency [%] |

| weight [Da] | time [min] | factor | MGN | control |
|---|---|---|---|---|
| 803.4 | 35.2 | 0.4 | 56 | 16 |
| 814.5 | 28.8 | 0.59 | 83 | 24 |
| 815.5 | 30.9 | −0.45 | 17 | 62 |
| 819.5 | 35.7 | 0.63 | 67 | 3 |
| 830.5 | 25.3 | 0.49 | 61 | 12 |
| 836.5 | 35 | 0.73 | 89 | 16 |
| 844.5 | 30.9 | 0.61 | 61 | 0 |
| 847.5 | 35.9 | 0.44 | 61 | 17 |
| 862.4 | 48.7 | 0.55 | 67 | 12 |
| 863.4 | 28.8 | 0.67 | 67 | 0 |
| 864.5 | 37.3 | 0.65 | 67 | 2 |
| 866.4 | 37.9 | 0.79 | 94 | 16 |
| 870.4 | 33.9 | 0.49 | 56 | 7 |
| 873.5 | 38.3 | 0.45 | 50 | 5 |
| 874.4 | 49.7 | 0.58 | 61 | 3 |
| 874.5 | 29.7 | 0.6 | 89 | 29 |
| 879.6 | 26.9 | −0.74 | 22 | 97 |
| 881.5 | 25.7 | 0.48 | 78 | 29 |
| 903.4 | 46.8 | −0.44 | 11 | 55 |
| 907.4 | 27.5 | 0.47 | 50 | 3 |
| 909.4 | 40.3 | 0.97 | 100 | 3 |
| 925.4 | 50.7 | 0.7 | 83 | 14 |
| 926.5 | 36.1 | −0.48 | 28 | 76 |
| 928.4 | 49.4 | −0.57 | 28 | 84 |
| 929.5 | 39.9 | −0.46 | 33 | 79 |
| 934.5 | 33.9 | 0.52 | 72 | 21 |
| 937.5 | 41.7 | 0.48 | 100 | 52 |
| 943.5 | 30 | 0.61 | 89 | 28 |
| 946.5 | 46.8 | −0.62 | 22 | 84 |
| 950.5 | 36 | 0.61 | 67 | 5 |
| 952.5 | 32 | 0.71 | 83 | 12 |
| 956.4 | 49.5 | −0.6 | 0 | 60 |
| 968.6 | 30.5 | 0.43 | 56 | 12 |
| 978.5 | 35 | 0.7 | 72 | 2 |
| 978.5 | 23.9 | 0.48 | 50 | 2 |
| 980.6 | 34.6 | −0.43 | 44 | 88 |
| 981.6 | 37.4 | −0.41 | 56 | 97 |
| 982.5 | 31.9 | 0.56 | 67 | 10 |
| 983.5 | 35.1 | 0.58 | 72 | 14 |
| 986.5 | 30.3 | 0.54 | 61 | 7 |
| 987.4 | 45.1 | 0.54 | 56 | 2 |
| 988.5 | 33.9 | 0.49 | 56 | 7 |
| 988.6 | 49.9 | −0.57 | 0 | 57 |
| 990.6 | 32 | 0.42 | 61 | 19 |
| 991.4 | 37.7 | 0.47 | 61 | 14 |
| 994.5 | 33.3 | 0.7 | 94 | 24 |
| 995.6 | 36.4 | 0.61 | 83 | 22 |
| 998.5 | 35.8 | 0.42 | 56 | 14 |
| 1000.5 | 34 | −0.67 | 28 | 95 |
| 1005.5 | 35 | 0.51 | 67 | 16 |
| 1006.4 | 35.7 | −0.5 | 28 | 78 |
| 1008.5 | 34.4 | −0.71 | 22 | 93 |
| 1010.6 | 50.8 | −0.58 | 6 | 64 |
| 1010.6 | 30.6 | −0.66 | 17 | 83 |
| 1013.4 | 39.3 | −0.52 | 0 | 52 |
| 1015.6 | 38.2 | 0.78 | 83 | 5 |
| 1017.4 | 36.6 | 0.63 | 83 | 21 |
| 1022.5 | 39.1 | 0.61 | 83 | 22 |
| 1028.6 | 37.8 | −0.76 | 22 | 98 |
| 1033.6 | 39.1 | 0.75 | 83 | 9 |
| 1038.6 | 34.4 | 0.58 | 72 | 14 |
| 1046.6 | 38.6 | −0.91 | 6 | 97 |
| 1047.6 | 30.4 | −0.46 | 39 | 84 |
| 1049.5 | 39.9 | 0.56 | 61 | 5 |
| 1051.5 | 36.2 | −0.47 | 17 | 64 |
| 1055.6 | 36.4 | 0.41 | 50 | 9 |
| 1058.6 | 21.5 | 0.56 | 56 | 0 |
| 1060.6 | 32 | 0.55 | 94 | 40 |
| 1071.5 | 38.7 | 0.54 | 56 | 2 |
| 1073.6 | 34.7 | 0.84 | 100 | 16 |
| 1075.6 | 29 | −0.47 | 17 | 64 |
| 1081.7 | 29.6 | −0.41 | 11 | 52 |
| 1090.5 | 36.2 | 0.56 | 83 | 28 |
| 1106.5 | 37.2 | 0.44 | 89 | 45 |
| 1108.6 | 29.8 | 0.55 | 94 | 40 |
| 1109.6 | 34.9 | −0.54 | 11 | 66 |
| 1110.4 | 46.9 | −0.6 | 11 | 71 |
| 1113.6 | 33.7 | 0.55 | 67 | 12 |
| 1114.5 | 37.4 | −0.48 | 6 | 53 |
| 1121.6 | 42.3 | −0.51 | 11 | 62 |
| 1122.5 | 50.2 | −0.43 | 22 | 66 |
| 1131.7 | 34.9 | 0.41 | 67 | 26 |
| 1132.6 | 36.7 | −0.45 | 44 | 90 |
| 1134.7 | 16.9 | 0.48 | 50 | 2 |
| 1135.6 | 42.7 | −0.66 | 0 | 66 |
| 1136.6 | 31.6 | 0.43 | 56 | 12 |
| 1139.6 | 32.2 | 0.54 | 61 | 7 |
| 1141.6 | 38 | −0.62 | 6 | 67 |
| 1150.6 | 35.8 | 0.46 | 94 | 48 |
| 1152.5 | 40.7 | 0.7 | 83 | 14 |
| 1157.6 | 28.5 | 0.6 | 89 | 29 |
| 1159.6 | 39 | −0.75 | 6 | 81 |
| 1163.7 | 38.1 | 0.58 | 61 | 3 |
| 1171.6 | 32.8 | 0.59 | 61 | 2 |
| 1181.6 | 37.8 | 0.48 | 89 | 41 |
| 1186.6 | 32 | 0.51 | 83 | 33 |
| 1191.6 | 50.5 | −0.41 | 50 | 91 |
| 1191.8 | 18.3 | 0.67 | 78 | 10 |
| 1198.8 | 29.2 | 0.63 | 94 | 31 |
| 1199.3 | 49.9 | −0.46 | 6 | 52 |
| 1203.7 | 24.7 | −0.57 | 0 | 57 |
| 1211.6 | 31.3 | 0.68 | 83 | 16 |
| 1212.7 | 30.6 | 0.75 | 83 | 9 |
| 1219.6 | 37.3 | 0.63 | 83 | 21 |
| 1220.6 | 30.2 | 0.51 | 83 | 33 |
| 1223.5 | 51.6 | −0.67 | 22 | 90 |
| 1224.7 | 33.6 | −0.67 | 33 | 100 |
| 1225.7 | 41.3 | 0.61 | 67 | 5 |
| 1226.7 | 41.6 | 0.47 | 50 | 3 |
| 1235.6 | 41.4 | −0.5 | 50 | 100 |
| 1236.7 | 34.8 | 0.41 | 67 | 26 |
| 1237.7 | 41.6 | −0.56 | 11 | 67 |
| 1246.7 | 30.5 | −0.54 | 11 | 66 |
| 1254.8 | 52.4 | −0.44 | 33 | 78 |
| 1264.6 | 45.9 | 0.52 | 61 | 9 |
| 1268.6 | 53.7 | −0.46 | 6 | 52 |
| 1269.7 | 39.8 | 0.42 | 61 | 19 |
| 1270.5 | 52.5 | −0.5 | 6 | 55 |
| 1270.6 | 25.7 | 0.61 | 67 | 5 |
| 1273.8 | 24.6 | 0.54 | 61 | 7 |
| 1274.6 | 38 | 0.42 | 56 | 14 |
| 1277.6 | 50 | −0.45 | 17 | 62 |
| 1279.7 | 38.3 | 0.79 | 94 | 16 |
| 1280.6 | 51.9 | −0.57 | 6 | 62 |
| 1283.9 | 28.9 | 0.61 | 67 | 5 |
| 1286 | 30.7 | 0.57 | 100 | 43 |
| 1292.5 | 53 | −0.64 | 22 | 86 |
| 1297.6 | 38.7 | 0.53 | 94 | 41 |
| 1302.7 | 31.8 | 0.51 | 83 | 33 |
| 1303.6 | 40.7 | −0.55 | 0 | 55 |
| 1308.6 | 53.6 | −0.4 | 44 | 84 |
| 1311.8 | 31.5 | 0.75 | 94 | 19 |
| 1319.9 | 34.8 | 0.64 | 83 | 19 |
| 1321.9 | 41.1 | −0.5 | 50 | 100 |
| 1324.2 | 40.5 | 0.52 | 61 | 9 |
| 1325.5 | 35.2 | 0.57 | 83 | 26 |
| 1331.7 | 35.4 | −0.48 | 11 | 59 |
| 1333.8 | 38.8 | 0.77 | 94 | 17 |
| 1335.4 | 53.4 | −0.52 | 0 | 52 |
| 1335.7 | 39.2 | 0.72 | 94 | 22 |
| 1338.7 | 47.2 | 0.76 | 78 | 2 |
| 1341.8 | 33.1 | 0.68 | 83 | 16 |
| 1350.7 | 50.3 | −0.5 | 0 | 50 |
| 1350.8 | 26.8 | 0.46 | 61 | 16 |
| 1353.7 | 39.3 | −0.51 | 39 | 90 |
| 1354.8 | 45.6 | 0.51 | 89 | 38 |
| 1355.7 | 36.3 | 0.4 | 56 | 16 |
| 1367.7 | 26.2 | 0.41 | 50 | 9 |
| 1370.8 | 33 | 0.41 | 50 | 9 |
| 1371.7 | 39.9 | 0.74 | 78 | 3 |
| 1371.8 | 19.3 | 0.74 | 100 | 26 |
| 1374.8 | 42.1 | 0.43 | 56 | 12 |
| 1377.7 | 25.4 | 0.52 | 56 | 3 |
| 1378.5 | 45.4 | −0.41 | 56 | 97 |
| 1381.1 | 32.3 | 0.58 | 94 | 36 |
| 1386 | 24.4 | 0.47 | 56 | 9 |
| 1389.8 | 19.5 | 0.54 | 89 | 34 |
| 1390.7 | 41.1 | 0.42 | 61 | 19 |
| 1395.5 | 25.4 | 0.43 | 50 | 7 |
| 1397.8 | 36.1 | 0.45 | 56 | 10 |
| 1398.9 | 30.5 | 0.81 | 94 | 14 |
| 1401.8 | 46.2 | −0.51 | 11 | 62 |
| 1404.9 | 29.4 | 0.53 | 100 | 47 |
| 1405.9 | 17.3 | 0.62 | 72 | 10 |
| 1408.9 | 26.8 | 0.56 | 67 | 10 |
| 1414.6 | 38.1 | 0.59 | 83 | 24 |
| 1415.7 | 33.3 | 0.45 | 50 | 5 |
| 1419.8 | 39.7 | 0.6 | 89 | 29 |
| 1423.6 | 22.3 | 0.61 | 67 | 5 |
| 1424.9 | 18.5 | 0.55 | 67 | 12 |
| 1426.8 | 38.7 | 0.58 | 67 | 9 |
| 1433 | 33.7 | 0.46 | 72 | 26 |
| 1439.6 | 25.4 | 0.41 | 50 | 9 |
| 1439.7 | 38.1 | 0.41 | 89 | 48 |
| 1442.8 | 33.3 | 0.63 | 83 | 21 |
| 1444.6 | 37.8 | 0.54 | 83 | 29 |
| 1448.8 | 30.3 | 0.45 | 78 | 33 |
| 1453.1 | 27.1 | 0.51 | 61 | 10 |
| 1462.7 | 53.6 | −0.48 | 50 | 98 |
| 1465.9 | 28.8 | 0.82 | 89 | 7 |
| 1472.1 | 31.2 | 0.75 | 89 | 14 |
| 1473.6 | 30.3 | 0.41 | 67 | 26 |
| 1474.9 | 16.9 | 0.71 | 83 | 12 |
| 1482 | 30.4 | 0.72 | 100 | 28 |
| 1482.8 | 36.3 | 0.47 | 83 | 36 |
| 1484 | 30.4 | 0.69 | 100 | 31 |
| 1487.7 | 41.4 | −0.58 | 33 | 91 |
| 1493.5 | 57 | 0.56 | 56 | 0 |
| 1497.9 | 26.7 | 0.57 | 78 | 21 |
| 1498.7 | 34.9 | 0.7 | 83 | 14 |
| 1499.9 | 30.6 | 0.6 | 94 | 34 |
| 1501.1 | 29.5 | 0.5 | 56 | 5 |
| 1502.8 | 28.8 | 0.41 | 72 | 31 |
| 1502.9 | 16.8 | 0.76 | 78 | 2 |
| 1504.4 | 30.5 | 0.41 | 72 | 31 |
| 1508.9 | 16.8 | 0.41 | 50 | 9 |
| 1510.1 | 39.5 | 0.48 | 89 | 41 |
| 1511.7 | 38.4 | 0.43 | 67 | 24 |
| 1518 | 26.8 | 0.46 | 94 | 48 |
| 1520.7 | 27.9 | 0.64 | 83 | 19 |
| 1522.5 | 26.4 | 0.42 | 61 | 19 |
| 1527.9 | 34.7 | 0.65 | 94 | 29 |
| 1529.7 | 54.1 | −0.62 | 22 | 84 |
| 1535 | 28.3 | 0.6 | 72 | 12 |
| 1537.9 | 31.5 | 0.56 | 83 | 28 |
| 1539.4 | 28.7 | 0.49 | 89 | 40 |
| 1540.7 | 29.8 | 0.68 | 83 | 16 |
| 1542.5 | 27.2 | 0.49 | 61 | 12 |
| 1543.8 | 34.9 | 0.54 | 83 | 29 |
| 1548.3 | 31.1 | 0.46 | 89 | 43 |
| 1552.3 | 35.5 | 0.41 | 100 | 59 |
| 1556.8 | 33.7 | 0.71 | 100 | 29 |
| 1558.1 | 23.4 | 0.52 | 61 | 9 |
| 1567 | 31.9 | 0.59 | 100 | 41 |
| 1567.6 | 53.9 | −0.5 | 6 | 55 |
| 1568.6 | 34.3 | 0.54 | 83 | 29 |
| 1573.8 | 40.4 | 0.5 | 94 | 45 |
| 1574.3 | 53.4 | −0.42 | 11 | 53 |
| 1574.8 | 33.9 | 0.68 | 89 | 21 |
| 1576.4 | 42.5 | −0.62 | 22 | 84 |
| 1578 | 52.5 | −0.48 | 50 | 98 |
| 1582.9 | 27.8 | 0.67 | 78 | 10 |
| 1588.4 | 47.9 | −0.61 | 11 | 72 |
| 1589.7 | 54.3 | −0.46 | 39 | 84 |
| 1595.4 | 31 | 0.61 | 78 | 17 |
| 1596.9 | 34 | 0.78 | 100 | 22 |
| 1604.3 | 21.6 | 0.42 | 56 | 14 |
| 1604.7 | 38.1 | 0.41 | 72 | 31 |
| 1605.7 | 53.3 | −0.46 | 28 | 74 |
| 1605.9 | 23.7 | 0.74 | 78 | 3 |
| 1607.7 | 41 | 0.49 | 61 | 12 |
| 1611.7 | 53.2 | −0.51 | 33 | 84 |
| 1612.8 | 36.8 | 0.67 | 100 | 33 |
| 1612.8 | 26.3 | 0.52 | 56 | 3 |
| 1613.9 | 36.3 | −0.46 | 39 | 84 |
| 1617.9 | 44.8 | −0.4 | 44 | 84 |
| 1619.7 | 53.9 | −0.45 | 17 | 62 |
| 1622 | 19.2 | 0.55 | 94 | 40 |
| 1629.5 | 32 | 0.42 | 61 | 19 |
| 1633.8 | 24.6 | 0.62 | 94 | 33 |
| 1635.2 | 27.8 | 0.43 | 78 | 34 |
| 1644 | 18.8 | 0.52 | 61 | 9 |
| 1652.3 | 28.6 | 0.6 | 100 | 40 |
| 1665 | 27.5 | 0.45 | 50 | 5 |
| 1666.3 | 23.3 | 0.48 | 89 | 41 |
| 1669.9 | 33.4 | 0.57 | 78 | 21 |
| 1676 | 25.3 | 0.66 | 78 | 12 |
| 1681.6 | 40 | 0.41 | 72 | 31 |
| 1686.8 | 38.2 | 0.7 | 94 | 24 |
| 1690.8 | 25.5 | 0.63 | 94 | 31 |
| 1692.4 | 30.4 | 0.46 | 61 | 16 |
| 1695.1 | 23.6 | 0.43 | 56 | 12 |
| 1699.1 | 41.9 | 0.7 | 94 | 24 |
| 1702.9 | 24.5 | 0.42 | 56 | 14 |
| 1706.8 | 21.5 | 0.51 | 67 | 16 |
| 1711 | 43.3 | −0.54 | 11 | 66 |
| 1713.4 | 24.6 | 0.42 | 61 | 19 |
| 1718.5 | 22.6 | 0.75 | 83 | 9 |
| 1723.3 | 26.5 | 0.49 | 67 | 17 |
| 1726 | 36.3 | 0.86 | 94 | 9 |
| 1729.2 | 26 | 0.75 | 89 | 14 |
| 1732 | 51.6 | −0.44 | 33 | 78 |
| 1734.6 | 27.2 | 0.47 | 78 | 31 |
| 1739.8 | 35.7 | 0.48 | 89 | 41 |
| 1744.1 | 34.3 | 0.67 | 100 | 33 |
| 1746.2 | 46.2 | −0.68 | 17 | 84 |
| 1747.7 | 50.8 | −0.57 | 0 | 57 |
| 1752.9 | 39.9 | 0.44 | 67 | 22 |
| 1763 | 24.4 | 0.72 | 94 | 22 |
| 1768.9 | 44.7 | −0.63 | 17 | 79 |
| 1770.4 | 45.4 | 0.46 | 94 | 48 |
| 1772.6 | 28.5 | 0.4 | 56 | 16 |
| 1774.6 | 36.5 | 0.61 | 78 | 17 |
| 1782.1 | 33 | 0.43 | 94 | 52 |
| 1786.9 | 35.9 | 0.55 | 72 | 17 |
| 1788.6 | 31 | 0.42 | 83 | 41 |
| 1791 | 25 | 0.58 | 67 | 9 |
| 1792 | 25.1 | 0.4 | 56 | 16 |
| 1793.6 | 28.3 | 0.67 | 72 | 5 |
| 1802.5 | 25.6 | 0.53 | 72 | 19 |
| 1804.7 | 34 | 0.45 | 100 | 55 |
| 1808.1 | 45.6 | 0.56 | 61 | 5 |
| 1810.1 | 31.8 | 0.64 | 83 | 19 |
| 1811.3 | 31.3 | 0.62 | 100 | 38 |
| 1813.4 | 54.7 | −0.42 | 11 | 53 |
| 1815.2 | 27.7 | 0.62 | 78 | 16 |
| 1819.9 | 24.1 | 0.7 | 83 | 14 |
| 1820.1 | 31.8 | 0.46 | 89 | 43 |
| 1821.2 | 18.2 | 0.61 | 67 | 5 |
| 1822.9 | 40.7 | −0.61 | 22 | 83 |
| 1824.3 | 37 | −0.57 | 22 | 79 |
| 1826.1 | 21.8 | 0.64 | 78 | 14 |
| 1831.9 | 41.5 | 0.41 | 50 | 9 |
| 1833.5 | 20.4 | 0.48 | 50 | 2 |
| 1837.6 | 38.2 | 0.41 | 72 | 31 |
| 1839.1 | 35.5 | 0.58 | 94 | 36 |
| 1847.8 | 57 | −0.65 | 28 | 93 |
| 1849.6 | 37.2 | −0.48 | 33 | 81 |
| 1851.2 | 31.6 | 0.51 | 89 | 38 |
| 1853 | 31.2 | 0.78 | 100 | 22 |
| 1853.6 | 46.7 | 0.47 | 50 | 3 |
| 1854.2 | 28.8 | 0.54 | 61 | 7 |
| 1856.8 | 56.3 | −0.52 | 17 | 69 |
| 1857.1 | 39 | 0.65 | 94 | 29 |
| 1859.4 | 22.8 | 0.63 | 67 | 3 |
| 1860.3 | 25.9 | 0.4 | 56 | 16 |
| 1863.8 | 57.5 | −0.54 | 39 | 93 |
| 1864.6 | 28.6 | 0.77 | 94 | 17 |
| 1867 | 31.8 | 0.69 | 100 | 31 |
| 1870.4 | 30.4 | 0.46 | 94 | 48 |
| 1870.5 | 16.1 | 0.45 | 50 | 5 |
| 1876.2 | 40.1 | 0.65 | 89 | 24 |
| 1878.7 | 49.9 | −0.52 | 28 | 79 |
| 1878.9 | 30.2 | 0.41 | 72 | 31 |
| 1880.3 | 57.4 | −0.44 | 11 | 55 |
| 1883 | 29.1 | −0.75 | 6 | 81 |
| 1885.7 | 57.5 | −0.67 | 28 | 95 |
| 1887.8 | 33.8 | 0.48 | 100 | 52 |
| 1889.8 | 46.4 | −0.58 | 39 | 97 |
| 1891.6 | 32.3 | 0.72 | 94 | 22 |
| 1894.9 | 22 | 0.75 | 94 | 19 |
| 1894.9 | 56 | −0.43 | 22 | 66 |
| 1896.8 | 53.3 | −0.5 | 6 | 55 |
| 1898.7 | 26.5 | 0.7 | 83 | 14 |
| 1904 | 27.5 | 0.59 | 61 | 2 |
| 1913.4 | 30.1 | 0.51 | 61 | 10 |
| 1913.9 | 53.9 | −0.4 | 39 | 79 |
| 1916.8 | 44.7 | −0.49 | 17 | 66 |
| 1920.7 | 30.6 | 0.55 | 100 | 45 |
| 1924.2 | 32.9 | 0.51 | 89 | 38 |
| 1931.4 | 26.6 | 0.43 | 56 | 12 |
| 1933.9 | 32.8 | −0.86 | 6 | 91 |
| 1934.2 | 16.1 | 0.87 | 89 | 2 |
| 1936.5 | 46.6 | −0.45 | 22 | 67 |
| 1936.7 | 32.8 | 0.71 | 100 | 29 |
| 1944.1 | 32.2 | 0.47 | 83 | 36 |
| 1944.2 | 47 | −0.75 | 11 | 86 |
| 1949.1 | 38.5 | −0.42 | 44 | 86 |
| 1950.9 | 34.5 | 0.58 | 89 | 31 |
| 1951.1 | 53 | −0.47 | 17 | 64 |
| 1966.3 | 25.1 | 0.84 | 94 | 10 |
| 1971.3 | 35.1 | 0.48 | 89 | 41 |
| 1971.5 | 18.9 | 0.61 | 67 | 5 |
| 1973.7 | 57.1 | −0.53 | 6 | 59 |
| 1977 | 25.5 | 0.46 | 94 | 48 |
| 1977.4 | 42.9 | −0.66 | 22 | 88 |
| 1982.9 | 32.2 | 0.65 | 89 | 24 |
| 1986.6 | 35.8 | 0.46 | 72 | 26 |
| 1988.9 | 28.8 | 0.61 | 78 | 17 |
| 1989.3 | 43.7 | 0.79 | 94 | 16 |
| 1990.8 | 47.3 | −0.66 | 17 | 83 |
| 2005.3 | 39.6 | 0.52 | 83 | 31 |
| 2011.3 | 29 | 0.47 | 56 | 9 |
| 2011.5 | 42 | 0.43 | 72 | 29 |
| 2013.8 | 45.3 | −0.51 | 17 | 67 |
| 2022.6 | 34.6 | 0.55 | 78 | 22 |
| 2025 | 24.2 | 0.58 | 67 | 9 |
| 2028.4 | 29.9 | 0.7 | 94 | 24 |
| 2030.4 | 31.7 | −0.64 | 17 | 81 |
| 2030.8 | 46.5 | −0.64 | 22 | 86 |
| 2032.1 | 30.6 | 0.42 | 56 | 14 |
| 2033.5 | 27.5 | 0.69 | 78 | 9 |
| 2035.6 | 30.9 | 0.41 | 67 | 26 |
| 2042 | 26.4 | 0.52 | 100 | 48 |
| 2042.5 | 40.7 | 0.63 | 83 | 21 |
| 2045.9 | 25.3 | 0.53 | 94 | 41 |
| 2047 | 45.4 | −0.48 | 50 | 98 |
| 2050.8 | 38.2 | 0.54 | 89 | 34 |
| 2052.5 | 38.7 | 0.41 | 67 | 26 |
| 2057.2 | 36.3 | 0.43 | 100 | 57 |
| 2065.3 | 20.9 | 0.69 | 72 | 3 |
| 2079.7 | 21.8 | 0.54 | 61 | 7 |
| 2092 | 26.7 | 0.75 | 89 | 14 |
| 2092.5 | 41.3 | 0.62 | 78 | 16 |
| 2093.1 | 25.3 | 0.47 | 56 | 9 |
| 2095.3 | 33.7 | 0.46 | 89 | 43 |
| 2099.2 | 36.9 | 0.69 | 94 | 26 |
| 2103.6 | 26.7 | 0.51 | 94 | 43 |
| 2105.4 | 32.5 | 0.64 | 78 | 14 |
| 2109.3 | 27.9 | 0.74 | 94 | 21 |
| 2116.3 | 20.3 | 0.51 | 67 | 16 |
| 2117.1 | 57.1 | −0.66 | 11 | 78 |
| 2121.1 | 43.1 | 0.4 | 61 | 21 |
| 2127.2 | 39.6 | 0.48 | 78 | 29 |
| 2129.5 | 35.1 | −0.8 | 17 | 97 |
| 2140.1 | 26.8 | 0.77 | 89 | 12 |
| 2144.3 | 22 | 0.67 | 94 | 28 |
| 2146.3 | 25.8 | 0.97 | 100 | 3 |
| 2152.7 | 29.5 | 0.75 | 94 | 19 |
| 2157.2 | 24.4 | 0.41 | 50 | 9 |
| 2163.4 | 27.6 | 0.56 | 67 | 10 |
| 2167.3 | 27.8 | 0.45 | 72 | 28 |
| 2172.5 | 36.7 | 0.46 | 94 | 48 |
| 2174.4 | 24.6 | 0.66 | 89 | 22 |
| 2178.5 | 21.4 | 0.69 | 78 | 9 |
| 2182.5 | 27.6 | 0.65 | 89 | 24 |
| 2197.9 | 29 | 0.48 | 67 | 19 |
| 2200.2 | 33.6 | 0.47 | 78 | 31 |
| 2205.6 | 23 | 0.42 | 56 | 14 |
| 2207.2 | 41.9 | 0.46 | 67 | 21 |
| 2210.7 | 25.7 | 0.72 | 94 | 22 |
| 2212.9 | 46.3 | −0.46 | 39 | 84 |
| 2217.7 | 41.9 | 0.6 | 94 | 34 |
| 2221.1 | 40.7 | −0.64 | 0 | 64 |
| 2223.5 | 22.6 | 0.75 | 83 | 9 |
| 2228.1 | 25.9 | 0.55 | 94 | 40 |
| 2230.1 | 22.8 | 0.52 | 56 | 3 |
| 2241 | 41.1 | 0.47 | 78 | 31 |
| 2241.1 | 22.7 | 0.63 | 94 | 31 |
| 2246.6 | 39.1 | 0.41 | 94 | 53 |
| 2253.1 | 22.4 | 0.54 | 56 | 2 |
| 2258.9 | 33.6 | 0.51 | 89 | 38 |
| 2264.4 | 34.8 | −0.44 | 17 | 60 |
| 2266 | 18.5 | 0.5 | 50 | 0 |
| 2273.5 | 22.4 | 0.42 | 56 | 14 |
| 2279.1 | 47.2 | −0.5 | 44 | 95 |
| 2279.5 | 34.8 | 0.46 | 94 | 48 |
| 2288.8 | 41.4 | 0.45 | 78 | 33 |
| 2288.9 | 27 | 0.48 | 67 | 19 |
| 2290.7 | 36.2 | 0.61 | 67 | 5 |
| 2291.1 | 21.9 | 0.67 | 72 | 5 |
| 2302.9 | 36.7 | 0.52 | 100 | 48 |
| 2308.9 | 26.2 | 0.63 | 83 | 21 |
| 2312.5 | 22.9 | 0.71 | 83 | 12 |
| 2322.5 | 47.1 | 0.61 | 67 | 5 |
| 2325.5 | 19.5 | 0.5 | 50 | 0 |
| 2334.2 | 41.2 | 0.45 | 50 | 5 |
| 2352.4 | 24.7 | 0.42 | 61 | 19 |
| 2356.3 | 24 | 0.68 | 83 | 16 |
| 2364.4 | 38.9 | 0.65 | 89 | 24 |
| 2367.7 | 43.2 | −0.66 | 17 | 83 |
| 2370.7 | 27.3 | 0.49 | 61 | 12 |
| 2380 | 39.6 | 0.62 | 94 | 33 |
| 2383.9 | 21.1 | 0.52 | 56 | 3 |
| 2389.2 | 34.4 | 0.41 | 89 | 48 |
| 2391.2 | 24.3 | 0.82 | 94 | 12 |
| 2396.5 | 34.6 | 0.49 | 61 | 12 |
| 2406.4 | 31.8 | 0.59 | 100 | 41 |
| 2409.1 | 41.9 | 0.48 | 89 | 41 |
| 2414.5 | 40.8 | 0.4 | 61 | 21 |
| 2421 | 28.7 | 0.65 | 94 | 29 |
| 2423.1 | 27.4 | 0.61 | 89 | 28 |
| 2426.5 | 38.5 | 0.63 | 94 | 31 |
| 2427.4 | 24 | 0.63 | 94 | 31 |
| 2429.9 | 39.3 | −0.48 | 28 | 76 |
| 2432.2 | 38.3 | 0.7 | 83 | 14 |
| 2435 | 21.6 | 0.54 | 56 | 2 |
| 2443.4 | 31.9 | −0.46 | 39 | 84 |
| 2446.2 | 24.7 | 0.44 | 61 | 17 |
| 2449.3 | 28.3 | 0.45 | 83 | 38 |
| 2451.7 | 35.5 | 0.44 | 83 | 40 |
| 2453.6 | 32 | 0.7 | 83 | 14 |
| 2453.8 | 20.4 | 0.56 | 56 | 0 |
| 2455.6 | 27.7 | 0.5 | 72 | 22 |
| 2461.1 | 40.5 | 0.58 | 72 | 14 |
| 2464 | 47.2 | −0.62 | 0 | 62 |
| 2465 | 22.8 | 0.89 | 94 | 5 |
| 2469.3 | 32.5 | 0.49 | 61 | 12 |
| 2471.7 | 23.8 | 0.53 | 72 | 19 |
| 2473.4 | 41.9 | 0.52 | 61 | 9 |
| 2475.5 | 22.3 | 0.49 | 61 | 12 |
| 2480.2 | 47.2 | −0.5 | 22 | 72 |
| 2483.8 | 19.6 | 0.55 | 72 | 17 |
| 2485.9 | 47.5 | 0.41 | 72 | 31 |
| 2490.7 | 26.7 | 0.56 | 83 | 28 |
| 2493.4 | 46 | −0.48 | 33 | 81 |
| 2493.6 | 24.6 | 0.86 | 100 | 14 |
| 2500.3 | 30.4 | −0.57 | 22 | 79 |
| 2502.9 | 33 | 0.46 | 61 | 16 |
| 2507.3 | 17.2 | 0.48 | 50 | 2 |
| 2518.7 | 38.9 | 0.59 | 83 | 24 |
| 2521.3 | 48.3 | 0.56 | 61 | 5 |
| 2522.9 | 24.4 | 0.63 | 83 | 21 |
| 2525.5 | 35.6 | −0.64 | 22 | 86 |
| 2527.3 | 40.8 | 0.6 | 72 | 12 |
| 2529.2 | 41.4 | −0.49 | 11 | 60 |
| 2535 | 37.7 | 0.55 | 94 | 40 |
| 2536.6 | 24.8 | 0.47 | 56 | 9 |
| 2540.5 | 25.5 | 0.79 | 89 | 10 |
| 2548.2 | 35.1 | −0.55 | 28 | 83 |
| 2553.7 | 24.7 | 0.44 | 61 | 17 |
| 2561.3 | 21.6 | 0.48 | 50 | 2 |
| 2566.4 | 22.2 | 0.65 | 72 | 7 |
| 2568.9 | 26.9 | 0.48 | 78 | 29 |
| 2570.5 | 57.1 | −0.44 | 33 | 78 |
| 2573.7 | 16.3 | 0.8 | 89 | 9 |
| 2576.2 | 25.4 | 0.46 | 67 | 21 |
| 2579.5 | 15.2 | 0.63 | 67 | 3 |
| 2584 | 43.8 | −0.69 | 28 | 97 |
| 2592.5 | 56.6 | −0.45 | 17 | 62 |
| 2593.4 | 25 | 0.4 | 56 | 16 |
| 2601.6 | 23.2 | 0.45 | 56 | 10 |
| 2607 | 47.6 | −0.52 | 22 | 74 |
| 2608.6 | 37.6 | 0.42 | 89 | 47 |
| 2614.1 | 22.5 | 0.61 | 78 | 17 |
| 2619.6 | 38.3 | 0.4 | 61 | 21 |
| 2619.7 | 22.9 | 0.63 | 67 | 3 |
| 2621.4 | 25.8 | 0.66 | 78 | 12 |
| 2627.4 | 44.8 | −0.41 | 50 | 91 |
| 2630.6 | 41.7 | 0.53 | 72 | 19 |
| 2639.6 | 45.2 | 0.5 | 56 | 5 |
| 2642.4 | 40.9 | −0.61 | 17 | 78 |
| 2644.1 | 32.5 | −0.6 | 33 | 93 |
| 2646.7 | 21.9 | 0.43 | 50 | 7 |
| 2654.3 | 37.2 | −0.54 | 11 | 66 |
| 2658.5 | 24.7 | −0.42 | 44 | 86 |
| 2660.8 | 27.1 | 0.59 | 78 | 19 |
| 2665.3 | 39.4 | 0.79 | 89 | 10 |
| 2666 | 23 | 0.45 | 56 | 10 |
| 2677.6 | 23.6 | 0.84 | 94 | 10 |
| 2687.9 | 28.2 | 0.5 | 56 | 5 |
| 2697.3 | 42.4 | −0.48 | 6 | 53 |
| 2698.4 | 32.1 | −0.52 | 28 | 79 |
| 2706.7 | 18.5 | 0.48 | 50 | 2 |
| 2707.2 | 34.1 | 0.45 | 72 | 28 |
| 2712.9 | 22.6 | 0.52 | 72 | 21 |
| 2713.2 | 41.3 | −0.57 | 6 | 62 |
| 2719.9 | 20.2 | 0.67 | 72 | 5 |
| 2733.4 | 34.6 | −0.42 | 44 | 86 |
| 2752.8 | 25.3 | 0.69 | 94 | 26 |
| 2758.5 | 40.9 | −0.43 | 28 | 71 |
| 2775.5 | 26.3 | 0.43 | 50 | 7 |
| 2780.4 | 28.3 | 0.7 | 83 | 14 |
| 2784.3 | 45.2 | 0.61 | 78 | 17 |
| 2790.3 | 26.8 | 0.62 | 78 | 16 |
| 2793.7 | 36.3 | 0.62 | 78 | 16 |
| 2809.1 | 37.2 | −0.61 | 17 | 78 |
| 2812.5 | 32.8 | 0.62 | 78 | 16 |
| 2823.3 | 39.9 | −0.43 | 44 | 88 |
| 2825.4 | 36.5 | −0.59 | 39 | 98 |
| 2830.9 | 33.2 | 0.75 | 94 | 19 |
| 2834.1 | 38.2 | −0.45 | 28 | 72 |
| 2841.6 | 37.1 | −0.45 | 28 | 72 |
| 2848.8 | 36.3 | −0.5 | 44 | 95 |
| 2854.4 | 43.8 | −0.41 | 56 | 97 |
| 2875.1 | 59.1 | 0.5 | 50 | 0 |
| 2883.6 | 28.9 | 0.44 | 61 | 17 |
| 2898.5 | 42.3 | −0.4 | 44 | 84 |
| 2902.9 | 42.1 | 0.55 | 67 | 12 |
| 2908.2 | 49.2 | −0.42 | 22 | 64 |
| 2912.9 | 57.5 | 0.5 | 50 | 0 |
| 2937.1 | 26.6 | 0.52 | 61 | 9 |
| 2945.1 | 22.6 | 0.52 | 61 | 9 |
| 2972.2 | 25.6 | 0.48 | 67 | 19 |
| 2973.7 | 34.9 | −0.66 | 22 | 88 |
| 2978 | 26.3 | −0.66 | 17 | 83 |
| 2978.3 | 41.7 | −0.52 | 28 | 79 |
| 2990.4 | 33.6 | −0.51 | 17 | 67 |
| 3007.5 | 30.5 | −0.45 | 22 | 67 |
| 3041.2 | 45 | −0.61 | 39 | 100 |
| 3044.8 | 48.6 | −0.52 | 22 | 74 |
| 3057.1 | 56.4 | −0.51 | 33 | 84 |
| 3058.8 | 35.5 | −0.42 | 44 | 86 |
| 3061.9 | 30.4 | 0.54 | 89 | 34 |
| 3077 | 28.4 | −0.55 | 28 | 83 |
| 3082.3 | 43.1 | −0.46 | 33 | 79 |
| 3098.8 | 42.6 | −0.44 | 56 | 100 |
| 3114.9 | 44.5 | −0.53 | 33 | 86 |
| 3121.4 | 42.5 | −0.56 | 44 | 100 |
| 3133.8 | 43.9 | −0.45 | 17 | 62 |
| 3139.4 | 43.7 | −0.61 | 17 | 78 |
| 3149.7 | 41.6 | −0.48 | 50 | 98 |
| 3152.6 | 38.2 | −0.61 | 39 | 100 |
| 3169 | 37.5 | −0.41 | 33 | 74 |
| 3177.4 | 22.3 | −0.5 | 22 | 72 |
| 3187.7 | 48.6 | −0.53 | 28 | 81 |
| 3190.9 | 28.8 | −0.42 | 22 | 64 |
| 3193.1 | 35.5 | −0.46 | 6 | 52 |
| 3205.8 | 28.3 | −0.64 | 22 | 86 |
| 3209.2 | 34.3 | −0.63 | 33 | 97 |
| 3219.5 | 20.2 | 0.7 | 83 | 14 |
| 3232.5 | 35.7 | −0.46 | 11 | 57 |
| 3255.8 | 42.9 | −0.62 | 22 | 84 |
| 3256.3 | 23.1 | 0.65 | 67 | 2 |
| 3258.6 | 36.3 | −0.52 | 17 | 69 |
| 3260.9 | 57.3 | −0.48 | 6 | 53 |
| 3262 | 31.5 | −0.58 | 28 | 86 |
| 3281 | 36.8 | −0.76 | 22 | 98 |
| 3290.9 | 36.9 | −0.6 | 33 | 93 |
| 3293.2 | 54.2 | −0.47 | 50 | 97 |
| 3295.8 | 38.4 | −0.65 | 33 | 98 |
| 3300.3 | 44.5 | 0.41 | 50 | 9 |
| 3303.2 | 38.6 | −0.79 | 6 | 84 |
| 3308.6 | 21.3 | 0.74 | 78 | 3 |
| 3309.7 | 43.6 | −0.56 | 17 | 72 |
| 3319.3 | 46.2 | −0.56 | 39 | 95 |
| 3320 | 26.7 | 0.42 | 56 | 14 |
| 3333.4 | 23.3 | −0.55 | 33 | 88 |
| 3334.6 | 41.7 | −0.64 | 22 | 86 |
| 3336.8 | 53.8 | −0.58 | 6 | 64 |
| 3337.4 | 36.2 | −0.49 | 39 | 88 |
| 3343.8 | 43.8 | −0.41 | 50 | 91 |
| 3372.2 | 32.5 | 0.51 | 83 | 33 |
| 3381.9 | 43.9 | −0.42 | 11 | 53 |
| 3398.9 | 44.5 | −0.41 | 50 | 91 |
| 3402.4 | 33.8 | −0.43 | 56 | 98 |
| 3405.7 | 37.8 | −0.82 | 17 | 98 |
| 3422.5 | 38.7 | −0.79 | 11 | 90 |
| 3433.3 | 44.5 | −0.43 | 56 | 98 |
| 3436 | 26.4 | −0.54 | 17 | 71 |
| 3442.8 | 42.5 | −0.44 | 56 | 100 |
| 3451.5 | 32.6 | −0.43 | 22 | 66 |
| 3479.3 | 48.5 | −0.83 | 17 | 100 |
| 3503.3 | 23.2 | −0.48 | 11 | 59 |
| 3530.9 | 36.8 | −0.59 | 22 | 81 |
| 3552 | 38.8 | −0.48 | 6 | 53 |
| 3578.2 | 32.5 | 0.58 | 72 | 14 |
| 3583.3 | 25.2 | −0.79 | 11 | 90 |
| 3589.5 | 39.1 | −0.62 | 28 | 90 |
| 3617.4 | 44.8 | −0.53 | 6 | 59 |
| 3631.2 | 33.1 | −0.6 | 11 | 71 |
| 3634.9 | 42.6 | −0.52 | 28 | 79 |
| 3669.7 | 36.7 | −0.47 | 17 | 64 |
| 3682.4 | 42.8 | −0.57 | 17 | 74 |
| 3686.1 | 32.6 | −0.72 | 11 | 83 |
| 3697.4 | 38.8 | −0.42 | 11 | 53 |
| 3701.8 | 43.4 | −0.72 | 0 | 72 |
| 3707 | 31.9 | −0.7 | 6 | 76 |
| 3719.6 | 44.7 | −0.55 | 28 | 83 |
| 3723.3 | 32.5 | −0.74 | 22 | 97 |
| 3735.8 | 43.9 | −0.68 | 11 | 79 |
| 3739.7 | 47.7 | −0.44 | 33 | 78 |
| 3760.8 | 25.9 | −0.52 | 17 | 69 |
| 3802.7 | 46.2 | −0.51 | 6 | 57 |
| 3816.7 | 32.2 | −0.54 | 11 | 66 |
| 3852.2 | 36.9 | −0.59 | 22 | 81 |
| 3871.7 | 42.9 | −0.42 | 22 | 64 |
| 3881.9 | 26.2 | 0.51 | 61 | 10 |
| 3946.9 | 33.1 | −0.65 | 28 | 93 |
| 3955.9 | 23.6 | 0.41 | 50 | 9 |
| 3969.6 | 31.3 | −0.75 | 11 | 86 |
| 3987 | 30.5 | −0.52 | 44 | 97 |
| 4006.7 | 45.9 | −0.43 | 22 | 66 |
| 4026.2 | 30.5 | −0.48 | 6 | 53 |
| 4044.7 | 31.2 | −0.75 | 11 | 86 |
| 4055.2 | 24.1 | 0.46 | 61 | 16 |
| 4102.1 | 41.9 | −0.5 | 0 | 50 |
| 4154.2 | 23.7 | 0.81 | 94 | 14 |
| 4170.6 | 46.1 | −0.46 | 6 | 52 |
| 4183.7 | 26.6 | 0.67 | 78 | 10 |
| 4241.2 | 24.4 | 0.86 | 100 | 14 |
| 4283.1 | 24.3 | 0.64 | 72 | 9 |
| 4290.8 | 41.1 | −0.66 | 22 | 88 |
| 4364.4 | 23.9 | −0.42 | 11 | 53 |
| 4369.9 | 27.1 | 0.53 | 94 | 41 |
| 4527.7 | 26 | 0.7 | 72 | 2 |
| 4565.8 | 25.1 | 0.41 | 50 | 9 |
| 4626.4 | 27.2 | 0.61 | 67 | 5 |
| 4654.8 | 38.8 | −0.41 | 11 | 52 |
| 4713.7 | 26.9 | 0.78 | 83 | 5 |
| 4719.5 | 39.3 | 0.5 | 50 | 0 |
| 4748.5 | 25.4 | −0.61 | 33 | 95 |
| 4772.1 | 28.9 | −0.41 | 11 | 52 |
| 4801.2 | 37.5 | −0.71 | 17 | 88 |
| 4827.1 | 27.3 | 0.82 | 83 | 2 |
| 4863.7 | 39.2 | −0.65 | 6 | 71 |
| 5112.9 | 33.1 | 0.5 | 50 | 0 |
| 5213.8 | 36.8 | −0.44 | 6 | 50 |
| 5229.1 | 39.9 | −0.41 | 11 | 52 |
| 5575.8 | 35.7 | −0.57 | 6 | 62 |
| 5829.7 | 20.8 | 0.59 | 61 | 2 |
| 5845.8 | 21.8 | 0.5 | 50 | 0 |
| 6106.5 | 27 | 0.56 | 56 | 0 |
| 6171.5 | 39.6 | −0.54 | 39 | 93 |
| 6212.4 | 30.6 | −0.59 | 0 | 59 |
| 6238.6 | 30.9 | −0.43 | 33 | 76 |
| 6400.9 | 23.4 | 0.7 | 72 | 2 |
| 7190.3 | 26.5 | 0.43 | 50 | 7 |
| 7284.9 | 40.7 | 0.5 | 50 | 0 |
| 7409.9 | 26.2 | 0.6 | 72 | 12 |
| 7556.6 | 26.2 | 0.73 | 89 | 16 |
| 7572.8 | 25.7 | 0.55 | 67 | 12 |
| 7885.4 | 20.9 | 0.56 | 61 | 5 |
| 8054.8 | 16.7 | 0.75 | 94 | 19 |
| 8216.9 | 16.8 | 0.45 | 50 | 5 |
| 8341.2 | 16.6 | 0.88 | 94 | 7 |
| 8371.2 | 15.8 | 0.61 | 61 | 0 |
| 8466.3 | 18 | 0.56 | 61 | 5 |
| 8518.7 | 15.7 | 0.76 | 78 | 2 |
| 8578.4 | 17 | 0.61 | 67 | 5 |
| 8653.1 | 17.2 | 0.55 | 67 | 12 |
| 8765.9 | 17.6 | 0.67 | 100 | 33 |
| 8803.1 | 16.2 | 0.47 | 50 | 3 |
| 8928.6 | 17 | 0.49 | 56 | 7 |
| 9060.7 | 23 | 0.62 | 72 | 10 |
| 9076 | 23 | 0.62 | 72 | 10 |
| 9182 | 17.1 | 0.8 | 89 | 9 |
| 9208 | 22.9 | 0.61 | 67 | 5 |
| 9223.1 | 22.8 | 0.76 | 83 | 7 |
| 9335.5 | 17.5 | 0.69 | 72 | 3 |
| 9465.1 | 23.3 | 0.61 | 61 | 0 |
| 9480.1 | 23.6 | 0.56 | 56 | 0 |
| 9621.9 | 19.2 | 0.58 | 67 | 9 |
| 9868.8 | 29.5 | −0.55 | 22 | 78 |
| 9933.5 | 18.4 | 0.52 | 56 | 3 |
| 9944.2 | 16.7 | 0.63 | 67 | 3 |
| 10046.3 | 18.1 | 0.81 | 100 | 19 |
| 10390.1 | 20.2 | 0.77 | 89 | 12 |
| 10518.8 | 20.9 | 0.53 | 72 | 19 |
| 10949.7 | 26.3 | 0.5 | 56 | 5 |

TABLE 10

| molecular | migration | discrimination | frequency [%] | |
|---|---|---|---|---|
| weight [Da] | time [min] | factor | MGN | FSGS + MCD |
| 814.5 | 28.8 | 0.41 | 83 | 42 |
| 819.5 | 35.7 | 0.47 | 67 | 19 |
| 863.4 | 28.8 | 0.44 | 67 | 23 |
| 864.5 | 37.3 | 0.44 | 67 | 23 |
| 879.6 | 26.9 | −0.59 | 22 | 81 |
| 909.4 | 40.3 | 0.5 | 100 | 50 |
| 928.4 | 49.4 | −0.41 | 28 | 69 |
| 1015.6 | 38.2 | 0.53 | 83 | 31 |
| 1017.4 | 36.6 | 0.49 | 83 | 35 |
| 1022.5 | 39.1 | 0.41 | 83 | 42 |
| 1073.6 | 34.7 | 0.42 | 100 | 58 |
| 1152.5 | 40.7 | 0.53 | 83 | 31 |
| 1224.7 | 33.6 | −0.44 | 33 | 77 |
| 1279.7 | 38.3 | 0.52 | 94 | 42 |
| 1283.9 | 28.9 | 0.44 | 67 | 23 |
| 1301.7 | 34 | 0.4 | 56 | 15 |
| 1319.9 | 34.8 | 0.41 | 83 | 42 |
| 1329.8 | 37.5 | 0.41 | 72 | 31 |
| 1341.8 | 33.1 | 0.53 | 83 | 31 |
| 1381.1 | 32.3 | 0.48 | 94 | 46 |
| 1404.9 | 29.4 | 0.46 | 100 | 54 |
| 1423.6 | 22.3 | 0.47 | 67 | 19 |
| 1433 | 33.7 | 0.41 | 72 | 31 |
| 1490.7 | 33.7 | 0.47 | 67 | 19 |
| 1543.8 | 34.9 | 0.41 | 83 | 42 |
| 1574.8 | 33.9 | 0.47 | 89 | 42 |
| 1595.4 | 31 | 0.43 | 78 | 35 |
| 1602.8 | 58.1 | −0.43 | 11 | 54 |
| 1605.9 | 23.7 | 0.55 | 78 | 23 |
| 1612.8 | 26.3 | 0.4 | 56 | 15 |
| 1726 | 36.3 | 0.41 | 94 | 54 |
| 1744.1 | 34.3 | 0.5 | 100 | 50 |
| 1768.9 | 44.7 | −0.56 | 17 | 73 |
| 1774.6 | 36.5 | 0.43 | 78 | 35 |
| 1799 | 28.8 | 0.41 | 72 | 31 |
| 1802.5 | 25.6 | 0.49 | 72 | 23 |
| 1839.1 | 35.5 | 0.41 | 94 | 54 |
| 1876.2 | 40.1 | 0.43 | 89 | 46 |
| 1883 | 29.1 | −0.44 | 6 | 50 |
| 1885.7 | 57.5 | −0.45 | 28 | 73 |
| 1898.7 | 26.5 | 0.41 | 83 | 42 |
| 1924.2 | 32.9 | 0.5 | 89 | 38 |
| 1933.9 | 32.8 | −0.52 | 6 | 58 |
| 1971.5 | 18.9 | 0.47 | 67 | 19 |
| 2011.3 | 29 | 0.4 | 56 | 15 |
| 2079.7 | 21.8 | 0.42 | 61 | 19 |
| 2109.3 | 27.9 | 0.44 | 94 | 50 |
| 2140.1 | 26.8 | 0.43 | 89 | 46 |
| 2152.7 | 29.5 | 0.41 | 94 | 54 |
| 2160.4 | 27.9 | 0.4 | 56 | 15 |
| 2274 | 37 | 0.42 | 50 | 8 |
| 2288.8 | 41.4 | 0.51 | 78 | 27 |
| 2292.4 | 35.3 | 0.43 | 78 | 35 |
| 2312.5 | 22.9 | 0.45 | 83 | 38 |
| 2332.4 | 35.4 | 0.44 | 67 | 23 |
| 2338.6 | 26 | 0.43 | 89 | 46 |
| 2341.2 | 26.3 | 0.44 | 56 | 12 |
| 2356.3 | 24 | 0.45 | 83 | 38 |
| 2367.7 | 43.2 | −0.45 | 17 | 62 |
| 2380 | 39.6 | 0.44 | 94 | 50 |
| 2391.2 | 24.3 | 0.41 | 94 | 54 |
| 2421 | 28.7 | 0.41 | 94 | 54 |
| 2451.7 | 35.5 | 0.45 | 83 | 38 |
| 2453.6 | 32 | 0.53 | 83 | 31 |
| 2453.8 | 20.4 | 0.44 | 56 | 12 |
| 2461.1 | 40.5 | 0.41 | 72 | 31 |
| 2469.3 | 32.5 | 0.46 | 61 | 15 |
| 2471.7 | 23.8 | 0.41 | 72 | 31 |
| 2500.3 | 30.4 | −0.43 | 22 | 65 |
| 2521.3 | 48.3 | 0.42 | 61 | 19 |
| 2525.5 | 35.6 | −0.51 | 22 | 73 |
| 2527.3 | 40.8 | 0.45 | 72 | 27 |
| 2639.6 | 45.2 | 0.4 | 56 | 15 |
| 2642.4 | 40.9 | −0.41 | 17 | 58 |
| 2665.3 | 39.4 | 0.54 | 89 | 35 |
| 2677.6 | 23.6 | 0.44 | 94 | 50 |
| 2784.3 | 45.2 | 0.51 | 78 | 27 |
| 2830.9 | 33.2 | 0.44 | 94 | 50 |
| 2912.9 | 57.5 | 0.42 | 50 | 8 |
| 3041.2 | 45 | −0.42 | 39 | 81 |
| 3205.8 | 28.3 | −0.43 | 22 | 65 |
| 3256.3 | 23.1 | 0.44 | 67 | 23 |
| 3313.8 | 31.6 | 0.51 | 78 | 27 |
| 3336.8 | 53.8 | −0.44 | 6 | 50 |
| 3479.3 | 48.5 | −0.56 | 17 | 73 |
| 3578.2 | 32.5 | 0.41 | 72 | 31 |
| 4183.7 | 26.6 | 0.43 | 78 | 35 |
| 4527.7 | 26 | 0.41 | 72 | 31 |
| 4827.1 | 27.3 | 0.53 | 83 | 31 |
| 5112.9 | 33.1 | 0.42 | 50 | 8 |
| 5829.7 | 20.8 | 0.46 | 61 | 15 |
| 6106.5 | 27 | 0.48 | 56 | 8 |
| 8341.2 | 16.6 | 0.48 | 94 | 46 |
| 8371.2 | 15.8 | 0.46 | 61 | 15 |
| 8466.3 | 18 | 0.46 | 61 | 15 |
| 8518.7 | 15.7 | 0.51 | 78 | 27 |
| 8578.4 | 17 | 0.47 | 67 | 19 |
| 9182 | 17.1 | 0.43 | 89 | 46 |
| 9944.2 | 16.7 | 0.44 | 67 | 23 |
| 10949.7 | 26.3 | 0.48 | 56 | 8 |

TABLE 11

| molecular | migration | discrimination | frequency [%] | |
|---|---|---|---|---|
| weight [Da] | time [min] | factor | IgA + MGN | control |
| 800.5 | 22.5 | −0.41 | 14 | 55 |
| 866.5 | 22 | 0.52 | 52 | 0 |
| 874.7 | 19.2 | 0.45 | 52 | 7 |
| 879.5 | 15.6 | −0.84 | 9 | 93 |
| 937.5 | 23 | 0.43 | 57 | 14 |
| 981.6 | 21.3 | −0.42 | 34 | 76 |
| 995.5 | 21.4 | 0.5 | 54 | 3 |
| 1010.6 | 16.3 | −0.57 | 5 | 62 |
| 1028.6 | 21.2 | −0.56 | 13 | 69 |
| 1046.5 | 21.5 | −0.74 | 9 | 83 |
| 1060.7 | 17.4 | 0.47 | 61 | 14 |
| 1134.6 | 20.4 | −0.59 | 41 | 100 |
| 1169.5 | 45.7 | −0.52 | 4 | 55 |
| 1194.7 | 22.9 | −0.68 | 32 | 100 |
| 1219.7 | 23.7 | 0.43 | 61 | 17 |
| 1224.7 | 18.1 | −0.58 | 11 | 69 |
| 1235.3 | 23.2 | −0.59 | 27 | 86 |
| 1250.6 | 23 | −0.52 | 48 | 100 |
| 1265.6 | 23 | −0.77 | 23 | 100 |
| 1285.8 | 18 | 0.54 | 57 | 3 |
| 1297.6 | 22.1 | 0.61 | 75 | 14 |
| 1321.7 | 23.6 | −0.46 | 20 | 66 |
| 1333.7 | 22.8 | 0.54 | 54 | 0 |
| 1335.5 | 21.1 | 0.45 | 63 | 17 |
| 1368.8 | 17 | 0.65 | 75 | 10 |
| 1386.9 | 17.2 | 0.75 | 79 | 3 |
| 1404.8 | 16 | 0.61 | 75 | 14 |
| 1424.4 | 32.1 | −0.57 | 43 | 100 |
| 1438.5 | 23.6 | −0.43 | 54 | 97 |
| 1448.6 | 17.5 | 0.57 | 64 | 7 |
| 1460.9 | 19.5 | 0.54 | 54 | 0 |
| 1482 | 17.7 | 0.65 | 71 | 7 |
| 1493.8 | 20 | 0.48 | 52 | 3 |
| 1499.9 | 17.6 | 0.77 | 88 | 10 |
| 1539.7 | 34.1 | −0.72 | 25 | 97 |
| 1567 | 19.2 | 0.59 | 63 | 3 |
| 1579.5 | 22.9 | −0.56 | 30 | 86 |
| 1585 | 18.1 | 0.77 | 91 | 14 |
| 1621.8 | 13.4 | 0.47 | 75 | 28 |
| 1644.4 | 20.8 | 0.63 | 63 | 0 |
| 1651.9 | 17.2 | 0.64 | 68 | 3 |
| 1698.1 | 18.4 | 0.72 | 96 | 24 |
| 1760 | 17.7 | 0.5 | 57 | 7 |
| 1805.1 | 19.8 | 0.5 | 50 | 0 |
| 1811.2 | 17.9 | 0.83 | 96 | 14 |
| 1819.7 | 20.4 | 0.52 | 59 | 7 |
| 1829.1 | 18 | 0.5 | 98 | 48 |
| 1851.1 | 17.8 | 0.77 | 84 | 7 |
| 1863.8 | 37 | −0.52 | 38 | 90 |
| 1867.2 | 18.6 | 0.82 | 86 | 3 |
| 1872.9 | 18.6 | 0.65 | 71 | 7 |
| 1878.5 | 17.3 | 0.52 | 59 | 7 |
| 1880.1 | 37.5 | −0.48 | 7 | 55 |
| 1895.1 | 16.2 | 0.63 | 77 | 14 |
| 1924.5 | 20.4 | 0.52 | 52 | 0 |
| 1943 | 19.5 | 0.79 | 86 | 7 |
| 1955 | 19.9 | 0.41 | 52 | 10 |
| 1977 | 12.7 | −0.57 | 5 | 62 |
| 2039.1 | 18.6 | −0.49 | 27 | 76 |
| 2042.1 | 17.7 | 0.68 | 75 | 7 |
| 2048 | 19.9 | −0.54 | 46 | 100 |
| 2057.3 | 19.1 | 0.71 | 71 | 0 |
| 2133.3 | 21.5 | 0.5 | 64 | 14 |
| 2147 | 19.5 | 0.54 | 68 | 14 |
| 2174.9 | 27.8 | −0.81 | 9 | 90 |
| 2233.1 | 18.1 | −0.48 | 11 | 59 |
| 2246.2 | 22.1 | 0.68 | 68 | 0 |
| 2249.1 | 18.7 | −0.42 | 23 | 66 |
| 2258.6 | 18.9 | 0.5 | 50 | 0 |
| 2279.5 | 22.5 | 0.5 | 54 | 3 |
| 2377.4 | 18.4 | −0.67 | 13 | 79 |
| 2389.2 | 18.6 | 0.59 | 66 | 7 |
| 2405.6 | 17.8 | 0.59 | 59 | 0 |
| 2427.1 | 16.4 | 0.84 | 95 | 10 |
| 2502.2 | 19.2 | 0.5 | 61 | 10 |
| 2518.7 | 18.8 | 0.61 | 64 | 3 |
| 2540.4 | 16 | 0.68 | 75 | 7 |
| 2562.9 | 19.1 | −0.52 | 38 | 90 |
| 2566.7 | 13.9 | 0.66 | 70 | 3 |
| 2608.3 | 21.8 | 0.54 | 75 | 21 |
| 2621.5 | 16.5 | 0.68 | 71 | 3 |
| 2649.6 | 28.9 | −0.5 | 13 | 62 |
| 2695.4 | 19.7 | −0.54 | 43 | 97 |
| 2742.3 | 23.7 | −0.61 | 36 | 97 |
| 2752.4 | 15.5 | 0.83 | 93 | 10 |
| 2755.3 | 23.6 | 0.68 | 71 | 3 |
| 2790.6 | 16.4 | 0.64 | 64 | 0 |
| 2799.7 | 20.4 | −0.45 | 48 | 93 |
| 2825 | 20.8 | −0.63 | 38 | 100 |
| 2838.7 | 20.3 | −0.57 | 36 | 93 |
| 2914.8 | 17 | 0.43 | 50 | 7 |
| 2936.8 | 16 | 0.7 | 70 | 0 |
| 3011.5 | 24.5 | −0.68 | 32 | 100 |
| 3013.3 | 16.8 | −0.42 | 20 | 62 |
| 3040.9 | 25.5 | −0.72 | 25 | 97 |
| 3098.3 | 24.8 | −0.51 | 11 | 62 |
| 3205.9 | 15.7 | −0.53 | 9 | 62 |
| 3209.4 | 19 | −0.67 | 16 | 83 |
| 3265.5 | 24 | −0.66 | 23 | 90 |
| 3281.1 | 27.3 | −0.56 | 38 | 93 |
| 3287.4 | 25.2 | −0.54 | 39 | 93 |
| 3303.5 | 28.7 | −0.49 | 23 | 72 |
| 3333.2 | 14.1 | −0.64 | 5 | 69 |
| 3359.6 | 26.2 | −0.72 | 4 | 76 |
| 3375.6 | 25.3 | −0.5 | 9 | 59 |
| 3385.7 | 21.2 | −0.61 | 32 | 93 |
| 3402.3 | 17.8 | −0.56 | 13 | 69 |
| 3405.3 | 21.6 | −0.75 | 18 | 93 |
| 3416.7 | 26.4 | −0.46 | 9 | 55 |
| 3432.5 | 25.6 | −0.69 | 7 | 76 |
| 3441.6 | 25.4 | −0.45 | 55 | 100 |
| 3457.8 | 25.4 | −0.45 | 55 | 100 |
| 3502.8 | 14.8 | −0.48 | 7 | 55 |
| 3582.9 | 14.2 | −0.72 | 7 | 79 |
| 3969.1 | 18.1 | −0.43 | 13 | 55 |
| 3987.1 | 17.3 | −0.63 | 27 | 90 |
| 4044.5 | 18 | −0.44 | 14 | 59 |
| 4054.8 | 15.5 | 0.45 | 59 | 14 |
| 4098.2 | 20.5 | −0.56 | 23 | 79 |
| 4153.7 | 14.7 | 0.58 | 71 | 14 |
| 4240.6 | 14.8 | 0.63 | 84 | 21 |
| 4290.3 | 23.4 | −0.65 | 7 | 72 |
| 4306.5 | 23.5 | −0.57 | 2 | 59 |
| 4369.2 | 15.5 | 0.44 | 75 | 31 |
| 4626 | 15.7 | 0.52 | 55 | 3 |
| 4712.9 | 15.8 | 0.72 | 79 | 7 |
| 4748 | 14.7 | −0.67 | 20 | 86 |
| 6171.4 | 23 | −0.7 | 16 | 86 |
| 6186.7 | 23.3 | −0.67 | 20 | 86 |
| 8764.7 | 12.8 | 0.46 | 88 | 41 |
| 9868.4 | 17.2 | −0.63 | 9 | 72 |
| 10044.3 | 13.1 | 0.65 | 89 | 24 |
| 10516.9 | 13.9 | 0.45 | 52 | 7 |
| 12719 | 22.6 | −0.44 | 25 | 69 |

TABLE 12

| | | | |
|---|---|---|---|
| molecular | migration | discrimination | frequency [%] |

| weight [Da] | time [min] | factor | IgA | control |
|---|---|---|---|---|
| 800.5 | 22.5 | −0.44 | 12 | 55 |
| 852.6 | 19.7 | 0.51 | 51 | 0 |
| 862.4 | 27.2 | 0.56 | 56 | 0 |
| 866.5 | 22 | 0.58 | 58 | 0 |
| 879.5 | 15.6 | −0.91 | 2 | 93 |
| 937.5 | 23 | 0.49 | 63 | 14 |
| 981.6 | 21.3 | −0.48 | 28 | 76 |
| 995.5 | 21.4 | 0.55 | 58 | 3 |
| 1008.5 | 20 | −0.44 | 12 | 55 |
| 1010.6 | 16.3 | −0.6 | 2 | 62 |
| 1028.6 | 21.2 | −0.57 | 12 | 69 |
| 1033.7 | 21.6 | 0.53 | 53 | 0 |
| 1046.5 | 21.5 | −0.8 | 2 | 83 |
| 1134.6 | 20.4 | −0.63 | 37 | 100 |
| 1144.7 | 25.8 | 0.48 | 51 | 3 |
| 1169.5 | 45.7 | −0.51 | 5 | 55 |
| 1181.6 | 23.7 | 0.48 | 65 | 17 |
| 1194.7 | 22.9 | −0.65 | 35 | 100 |
| 1219.7 | 23.7 | 0.55 | 72 | 17 |
| 1224.7 | 18.1 | −0.64 | 5 | 69 |
| 1235.3 | 23.2 | −0.61 | 26 | 86 |
| 1250.6 | 23 | −0.49 | 51 | 100 |
| 1265.6 | 23 | −0.79 | 21 | 100 |
| 1297.6 | 22.1 | 0.63 | 77 | 14 |
| 1321.7 | 23.6 | −0.52 | 14 | 66 |
| 1333.7 | 22.8 | 0.63 | 63 | 0 |
| 1335.5 | 21.1 | 0.55 | 72 | 17 |
| 1368.8 | 17 | 0.64 | 74 | 10 |
| 1386.9 | 17.2 | 0.71 | 74 | 3 |
| 1404.8 | 16 | 0.58 | 72 | 14 |
| 1424.4 | 32.1 | −0.49 | 51 | 100 |
| 1448.6 | 17.5 | 0.54 | 60 | 7 |
| 1482 | 17.7 | 0.61 | 67 | 7 |
| 1493.8 | 20 | 0.55 | 58 | 3 |
| 1499.9 | 17.6 | 0.76 | 86 | 10 |
| 1539.7 | 34.1 | −0.66 | 30 | 97 |
| 1567 | 19.2 | 0.5 | 53 | 3 |
| 1573.8 | 27.8 | 0.49 | 77 | 28 |
| 1579.5 | 22.9 | −0.65 | 21 | 86 |
| 1585 | 18.1 | 0.79 | 93 | 14 |
| 1621.8 | 13.4 | 0.47 | 74 | 28 |
| 1644.4 | 20.8 | 0.63 | 63 | 0 |
| 1651.9 | 17.2 | 0.62 | 65 | 3 |
| 1689.7 | 32.2 | 0.47 | 53 | 7 |
| 1698.1 | 18.4 | 0.74 | 98 | 24 |
| 1811.2 | 17.9 | 0.84 | 98 | 14 |
| 1819.7 | 20.4 | 0.47 | 53 | 7 |
| 1829.1 | 18 | 0.49 | 98 | 48 |
| 1851.1 | 17.8 | 0.74 | 81 | 7 |
| 1863.8 | 37 | −0.43 | 47 | 90 |
| 1867.2 | 18.6 | 0.8 | 84 | 3 |
| 1872.9 | 18.6 | 0.63 | 70 | 7 |
| 1878.5 | 17.3 | 0.49 | 56 | 7 |
| 1880.1 | 37.5 | −0.48 | 7 | 55 |
| 1895.1 | 16.2 | 0.58 | 72 | 14 |
| 1924.5 | 20.4 | 0.51 | 51 | 0 |
| 1943 | 19.5 | 0.79 | 86 | 7 |
| 1955 | 19.9 | 0.43 | 53 | 10 |
| 1977 | 12.7 | −0.57 | 5 | 62 |
| 2039.1 | 18.6 | −0.53 | 23 | 76 |
| 2042.1 | 17.7 | 0.7 | 77 | 7 |
| 2048 | 19.9 | −0.6 | 40 | 100 |
| 2057.3 | 19.1 | 0.67 | 67 | 0 |
| 2133.3 | 21.5 | 0.56 | 70 | 14 |
| 2147 | 19.5 | 0.44 | 58 | 14 |
| 2174.9 | 27.8 | −0.8 | 9 | 90 |
| 2233.1 | 18.1 | −0.52 | 7 | 59 |
| 2246.2 | 22.1 | 0.79 | 79 | 0 |
| 2249.1 | 18.7 | −0.49 | 16 | 66 |
| 2279.5 | 22.5 | 0.5 | 53 | 3 |
| 2377.4 | 18.4 | −0.79 | 0 | 79 |
| 2389.2 | 18.6 | 0.63 | 70 | 7 |
| 2405.6 | 17.8 | 0.58 | 58 | 0 |
| 2427.1 | 16.4 | 0.83 | 93 | 10 |
| 2483.4 | 21.6 | −0.47 | 12 | 59 |
| 2502.2 | 19.2 | 0.57 | 67 | 10 |
| 2518.7 | 18.8 | 0.52 | 56 | 3 |
| 2540.4 | 16 | 0.68 | 74 | 7 |
| 2562.9 | 19.1 | −0.52 | 37 | 90 |
| 2566.7 | 13.9 | 0.71 | 74 | 3 |
| 2608.3 | 21.8 | 0.65 | 86 | 21 |
| 2621.5 | 16.5 | 0.69 | 72 | 3 |
| 2649.6 | 28.9 | −0.5 | 12 | 62 |
| 2695.4 | 19.7 | −0.59 | 37 | 97 |
| 2742.3 | 23.7 | −0.62 | 35 | 97 |
| 2752.4 | 15.5 | 0.85 | 95 | 10 |
| 2755.3 | 23.6 | 0.8 | 84 | 3 |
| 2761.3 | 17.7 | −0.4 | 12 | 52 |
| 2790.6 | 16.4 | 0.6 | 60 | 0 |
| 2799.7 | 20.4 | −0.42 | 51 | 93 |
| 2825 | 20.8 | −0.67 | 33 | 100 |
| 2838.7 | 20.3 | −0.58 | 35 | 93 |
| 2936.8 | 16 | 0.67 | 67 | 0 |
| 3011.5 | 24.5 | −0.63 | 37 | 100 |
| 3013.3 | 16.8 | −0.48 | 14 | 62 |
| 3040.9 | 25.5 | −0.69 | 28 | 97 |
| 3098.3 | 24.8 | −0.57 | 5 | 62 |
| 3205.9 | 15.7 | −0.57 | 5 | 62 |
| 3209.4 | 19 | −0.73 | 9 | 83 |
| 3265.5 | 24 | −0.69 | 21 | 90 |
| 3281.1 | 27.3 | −0.56 | 37 | 93 |
| 3287.4 | 25.2 | −0.47 | 47 | 93 |
| 3303.5 | 28.7 | −0.49 | 23 | 72 |
| 3333.2 | 14.1 | −0.67 | 2 | 69 |
| 3359.6 | 26.2 | −0.71 | 5 | 76 |
| 3375.6 | 25.3 | −0.54 | 5 | 59 |
| 3385.7 | 21.2 | −0.56 | 37 | 93 |
| 3402.3 | 17.8 | −0.57 | 12 | 69 |
| 3405.3 | 21.6 | −0.79 | 14 | 93 |
| 3416.7 | 26.4 | −0.44 | 12 | 55 |
| 3432.5 | 25.6 | −0.67 | 9 | 76 |
| 3502.8 | 14.8 | −0.53 | 2 | 55 |
| 3582.9 | 14.2 | −0.72 | 7 | 79 |
| 3841.4 | 14.9 | −0.44 | 42 | 86 |
| 3969.1 | 18.1 | −0.41 | 14 | 55 |
| 3987.1 | 17.3 | −0.62 | 28 | 90 |
| 4044.5 | 18 | −0.42 | 16 | 59 |
| 4098.2 | 20.5 | −0.51 | 28 | 79 |
| 4153.7 | 14.7 | 0.51 | 65 | 14 |
| 4240.6 | 14.8 | 0.61 | 81 | 21 |
| 4290.3 | 23.4 | −0.63 | 9 | 72 |
| 4306.5 | 23.5 | −0.56 | 2 | 59 |
| 4712.9 | 15.8 | 0.68 | 74 | 7 |
| 4748 | 14.7 | −0.68 | 19 | 86 |
| 6171.4 | 23 | −0.68 | 19 | 86 |
| 6186.7 | 23.3 | −0.65 | 21 | 86 |
| 8764.7 | 12.8 | 0.45 | 86 | 41 |
| 9868.4 | 17.2 | −0.63 | 9 | 72 |
| 10044.3 | 13.1 | 0.64 | 88 | 24 |

TABLE 13

| | | | |
|---|---|---|---|
| molecular | migration time | discrimination | frequency [%] |

| | | | | |
|---|---|---|---|---|
| weight [Da] | [min] | factor | IgA | MGN |

| | | | | |
|---|---|---|---|---|
| 816.7 | 16 | −0.47 | 7 | 54 |
| 852.6 | 19.7 | 0.51 | 51 | 0 |
| 862.4 | 27.2 | 0.56 | 56 | 0 |
| 874.7 | 19.2 | −0.43 | 42 | 85 |
| 943.6 | 17.5 | −0.59 | 26 | 85 |
| 977.6 | 18 | −0.5 | 12 | 62 |
| 994.5 | 18.3 | −0.52 | 2 | 54 |
| 1004.6 | 22.4 | −0.49 | 5 | 54 |
| 1040.4 | 19.5 | −0.59 | 2 | 62 |
| 1060.7 | 17.4 | −0.41 | 51 | 92 |
| 1099.8 | 17.5 | −0.57 | 28 | 85 |
| 1108.6 | 17.3 | −0.5 | 42 | 92 |
| 1143.6 | 30.2 | 0.45 | 60 | 15 |
| 1157.5 | 31.6 | 0.47 | 63 | 15 |
| 1179.6 | 31.6 | 0.49 | 72 | 23 |
| 1186.7 | 19.7 | −0.49 | 5 | 54 |
| 1195.6 | 31.4 | 0.64 | 79 | 15 |
| 1198.8 | 17.7 | −0.49 | 28 | 77 |
| 1203.7 | 19.3 | 0.45 | 60 | 15 |
| 1217.6 | 30.1 | 0.78 | 93 | 15 |
| 1219.7 | 23.7 | 0.49 | 72 | 23 |
| 1239.6 | 30.6 | 0.75 | 91 | 15 |
| 1252.7 | 20.5 | −0.81 | 12 | 92 |
| 1255.6 | 29.4 | 0.49 | 72 | 23 |
| 1261.5 | 30.1 | 0.51 | 74 | 23 |
| 1268.4 | 23 | −0.51 | 26 | 77 |
| 1270.7 | 16.6 | −0.47 | 7 | 54 |
| 1285.8 | 18 | −0.46 | 47 | 92 |
| 1287.6 | 18.8 | −0.55 | 14 | 69 |
| 1311.8 | 17.9 | −0.46 | 23 | 69 |
| 1335.5 | 21.1 | 0.41 | 72 | 31 |
| 1340.6 | 16.7 | −0.52 | 9 | 62 |
| 1343.1 | 18.5 | −0.48 | 21 | 69 |
| 1350.8 | 19 | −0.48 | 14 | 62 |
| 1365.7 | 21.2 | −0.62 | 7 | 69 |
| 1377.8 | 17.1 | −0.47 | 7 | 54 |
| 1381 | 19.8 | −0.62 | 7 | 69 |
| 1402.4 | 18.7 | −0.52 | 2 | 54 |
| 1405.8 | 31.3 | 0.54 | 70 | 15 |
| 1424.8 | 14.7 | −0.5 | 12 | 62 |
| 1446.7 | 32.7 | 0.44 | 67 | 23 |
| 1455.8 | 20.7 | −0.7 | 7 | 77 |
| 1464.1 | 21.3 | −0.55 | 7 | 62 |
| 1465.7 | 19.4 | −0.67 | 2 | 69 |
| 1470.8 | 17.3 | −0.47 | 7 | 54 |
| 1472 | 21.3 | −0.53 | 16 | 69 |
| 1484.5 | 17.5 | −0.73 | 12 | 85 |
| 1486.4 | 19.5 | −0.58 | 12 | 69 |
| 1514.6 | 19.4 | −0.49 | 5 | 54 |
| 1519.1 | 13.4 | −0.5 | 12 | 62 |
| 1523.7 | 33 | 0.65 | 95 | 31 |
| 1539.6 | 21.8 | −0.62 | 30 | 92 |
| 1545.8 | 33.5 | 0.61 | 84 | 23 |
| 1556.7 | 19.6 | −0.85 | 0 | 85 |
| 1561.8 | 33.3 | 0.55 | 93 | 38 |
| 1561.9 | 19.8 | −0.57 | 5 | 62 |
| 1573.8 | 27.8 | 0.61 | 77 | 15 |
| 1579.5 | 22.9 | −0.41 | 21 | 62 |
| 1596.9 | 18.3 | −0.6 | 9 | 69 |
| 1603 | 19.1 | −0.55 | 7 | 62 |
| 1611.6 | 32.9 | 0.51 | 51 | 0 |
| 1627.6 | 19.6 | −0.52 | 2 | 54 |
| 1637.8 | 19.3 | −0.6 | 9 | 69 |
| 1651.8 | 34 | 0.59 | 74 | 15 |
| 1707.6 | 19.7 | −0.42 | 12 | 54 |
| 1726 | 20.1 | −0.6 | 9 | 69 |
| 1775.9 | 20.8 | −0.42 | 12 | 54 |
| 1782.3 | 18.9 | −0.42 | 35 | 77 |
| 1787.7 | 19.3 | −0.48 | 14 | 62 |
| 1791.3 | 19.3 | −0.56 | 21 | 77 |
| 1793.1 | 23 | −0.61 | 16 | 77 |
| 1799.3 | 18.7 | −0.54 | 0 | 54 |
| 1845.3 | 20.1 | −0.68 | 9 | 77 |
| 1876.8 | 19.2 | −0.52 | 33 | 85 |
| 1887.6 | 21.5 | −0.45 | 9 | 54 |
| 1891.2 | 18.7 | −0.59 | 2 | 62 |
| 1936.5 | 21.6 | −0.46 | 23 | 69 |
| 1968.5 | 27.3 | −0.5 | 12 | 62 |
| 1971.3 | 22.2 | −0.5 | 12 | 62 |
| 1988.9 | 18.9 | −0.42 | 12 | 54 |
| 2014.9 | 18.1 | −0.69 | 23 | 92 |
| 2025.9 | 21.7 | −0.49 | 5 | 54 |
| 2063.8 | 17.7 | −0.49 | 51 | 100 |
| 2085.9 | 21 | −0.45 | 9 | 54 |
| 2113 | 18.6 | −0.42 | 12 | 54 |
| 2129.1 | 16.6 | −0.57 | 5 | 62 |
| 2135.3 | 18.1 | −0.68 | 9 | 77 |
| 2147 | 19.5 | −0.42 | 58 | 100 |
| 2154.1 | 21.2 | −0.44 | 33 | 77 |
| 2159 | 23.1 | −0.47 | 37 | 85 |
| 2166.6 | 20.1 | −0.48 | 14 | 62 |
| 2171.7 | 21.1 | −0.41 | 21 | 62 |
| 2178.4 | 14.9 | −0.52 | 9 | 62 |
| 2183.5 | 16.3 | −0.47 | 7 | 54 |
| 2229.5 | 20.2 | −0.7 | 7 | 77 |
| 2246.2 | 22.1 | 0.48 | 79 | 31 |
| 2290.5 | 20.1 | −0.41 | 21 | 62 |
| 2292.2 | 20.6 | −0.47 | 7 | 54 |
| 2309.7 | 16 | −0.52 | 2 | 54 |
| 2325.2 | 18.2 | −0.53 | 16 | 69 |
| 2361.6 | 17.3 | −0.49 | 5 | 54 |
| 2377.4 | 18.4 | −0.54 | 0 | 54 |
| 2407.5 | 19.3 | −0.45 | 9 | 54 |
| 2421.1 | 16.1 | −0.62 | 0 | 62 |
| 2464.4 | 19.8 | −0.69 | 23 | 92 |
| 2466.6 | 17 | −0.48 | 21 | 69 |
| 2475.8 | 18.8 | −0.64 | 21 | 85 |
| 2483.4 | 21.6 | −0.73 | 12 | 85 |
| 2493.3 | 14.9 | −0.57 | 28 | 85 |
| 2522.4 | 16.3 | −0.41 | 28 | 69 |
| 2547.1 | 17.9 | −0.55 | 14 | 69 |
| 2553.7 | 17.7 | −0.72 | 5 | 77 |
| 2573.5 | 20.3 | −0.45 | 16 | 62 |
| 2586.7 | 18.5 | −0.59 | 2 | 62 |
| 2599.2 | 21 | −0.68 | 9 | 77 |
| 2608.3 | 21.8 | 0.48 | 86 | 38 |
| 2669.1 | 18.4 | −0.52 | 2 | 54 |
| 2676 | 20.6 | −0.55 | 14 | 69 |
| 2681.5 | 20.1 | −0.42 | 12 | 54 |
| 2684.2 | 19.4 | −0.55 | 7 | 62 |
| 2687.5 | 19.2 | −0.59 | 2 | 62 |
| 2755.3 | 23.6 | 0.53 | 84 | 31 |
| 2807.6 | 17.3 | −0.47 | 7 | 54 |
| 2810.5 | 18.1 | −0.5 | 12 | 62 |
| 2831.2 | 19 | −0.43 | 19 | 62 |
| 2847.7 | 16.5 | −0.5 | 35 | 85 |
| 2914.8 | 17 | −0.45 | 40 | 85 |
| 2959.7 | 16.6 | −0.47 | 7 | 54 |
| 3030 | 16.7 | −0.44 | 26 | 69 |
| 3062.1 | 17.9 | −0.54 | 23 | 77 |
| 3441.6 | 25.4 | 0.62 | 70 | 8 |
| 3478.6 | 27.6 | 0.78 | 86 | 8 |
| 3495.5 | 25.8 | 0.71 | 79 | 8 |
| 3841.4 | 14.9 | −0.43 | 42 | 85 |
| 4183.6 | 16.3 | −0.45 | 9 | 54 |
| 4479.4 | 14.8 | −0.45 | 9 | 54 |
| 4483.2 | 16.2 | −0.47 | 7 | 54 |
| 4527.6 | 16.3 | −0.5 | 12 | 62 |
| 4566.6 | 16.6 | −0.52 | 2 | 54 |
| 4594.4 | 14.2 | −0.45 | 9 | 54 |
| 8053.8 | 12.9 | −0.44 | 26 | 69 |

TABLE 14

| | | |
|---|---|---|
| mass | CE_t | frequency [%] |

| [Da] | [min] | control | FSGS | MCD | MGN |
|---|---|---|---|---|---|
| 3012.09 | 39.45 | 100 | 60 | 94 | 72 |
| 1539.67 | 50.20 | 100 | 90 | 100 | 89 |
| 2249.19 | 33.82 | 100 | 80 | 100 | 83 |
| 3152.55 | 38.22 | 100 | 60 | 69 | 39 |
| 3360.09 | 44.33 | 100 | 60 | 81 | 72 |
| 3001.97 | 48.35 | 100 | 100 | 100 | 89 |
| 2257.19 | 46.60 | 100 | 60 | 81 | 67 |
| 2563.42 | 32.22 | 100 | 90 | 75 | 72 |
| 2158.98 | 46.70 | 100 | 90 | 88 | 89 |
| 3287.97 | 43.92 | 100 | 90 | 100 | 89 |
| 3385.76 | 36.60 | 100 | 100 | 81 | 72 |
| 3271.80 | 44.05 | 100 | 80 | 94 | 61 |
| 2007.69 | 33.06 | 100 | 90 | 88 | 89 |
| 1194.61 | 39.46 | 100 | 70 | 88 | 83 |
| 1265.62 | 40.37 | 100 | 70 | 94 | 72 |
| 1435.69 | 39.91 | 100 | 90 | 94 | 89 |
| 1261.53 | 49.63 | 100 | 80 | 88 | 61 |
| 1438.56 | 37.63 | 100 | 100 | 100 | 100 |
| 1446.50 | 52.53 | 100 | 80 | 94 | 67 |
| 3265.77 | 42.28 | 100 | 80 | 75 | 78 |
| 3121.36 | 42.46 | 100 | 50 | 75 | 44 |
| 1911.14 | 37.40 | 100 | 90 | 100 | 94 |
| 1321.91 | 41.10 | 100 | 70 | 69 | 50 |
| 2695.49 | 35.27 | 100 | 70 | 88 | 78 |
| 1235.59 | 41.42 | 100 | 60 | 69 | 50 |
| 2799.94 | 37.08 | 100 | 80 | 88 | 83 |
| 2169.75 | 39.56 | 100 | 100 | 100 | 94 |
| 1224.74 | 33.57 | 100 | 50 | 94 | 33 |
| 1451.67 | 41.11 | 100 | 100 | 100 | 83 |
| 3479.32 | 48.53 | 100 | 70 | 75 | 17 |
| 2649.94 | 45.91 | 100 | 90 | 75 | 78 |
| 2687.36 | 41.87 | 100 | 60 | 75 | 61 |
| 3458.52 | 44.64 | 100 | 90 | 94 | 72 |
| 3442.84 | 42.54 | 100 | 80 | 94 | 56 |
| 2048.19 | 33.07 | 100 | 40 | 81 | 61 |
| 2679.46 | 34.99 | 100 | 50 | 94 | 67 |
| 2227.34 | 38.28 | 100 | 70 | 62 | 83 |
| 1239.52 | 50.23 | 100 | 100 | 100 | 78 |
| 3098.80 | 42.63 | 100 | 60 | 81 | 56 |
| 2839.07 | 35.41 | 100 | 80 | 88 | 83 |
| 3417.12 | 45.12 | 100 | 70 | 81 | 61 |
| 3426.20 | 42.48 | 100 | 70 | 88 | 72 |
| 3041.16 | 45.04 | 100 | 80 | 81 | 39 |
| 1508.70 | 41.26 | 98 | 90 | 94 | 61 |
| 1462.67 | 53.58 | 98 | 70 | 88 | 50 |
| 3280.96 | 36.76 | 98 | 20 | 50 | 22 |
| 1877.33 | 29.62 | 98 | 100 | 100 | 100 |
| 2742.25 | 42.25 | 98 | 90 | 75 | 89 |
| 3092.71 | 43.86 | 98 | 90 | 81 | 78 |
| 2196.66 | 45.45 | 98 | 80 | 100 | 89 |
| 6187.55 | 39.78 | 98 | 60 | 94 | 67 |
| 2825.42 | 36.54 | 98 | 50 | 88 | 39 |
| 1255.55 | 49.81 | 98 | 100 | 100 | 78 |
| 2717.56 | 34.43 | 98 | 60 | 69 | 61 |
| 3149.67 | 41.62 | 98 | 70 | 88 | 50 |
| 1195.53 | 51.76 | 98 | 70 | 94 | 83 |
| 3496.02 | 43.85 | 98 | 90 | 94 | 61 |
| 1561.69 | 54.17 | 98 | 90 | 100 | 83 |
| 1250.63 | 41.97 | 98 | 70 | 100 | 89 |
| 3295.77 | 38.36 | 98 | 50 | 50 | 33 |
| 3405.68 | 37.84 | 98 | 40 | 62 | 17 |
| 1578.01 | 52.53 | 98 | 70 | 75 | 50 |
| 1134.58 | 37.11 | 98 | 90 | 94 | 94 |
| 1028.57 | 37.79 | 98 | 40 | 69 | 22 |
| 876.41 | 48.89 | 98 | 70 | 38 | 67 |
| 2205.03 | 36.94 | 98 | 70 | 88 | 83 |
| 3402.40 | 33.83 | 98 | 50 | 94 | 56 |
| 2377.60 | 32.06 | 98 | 80 | 94 | 83 |
| 2175.03 | 44.27 | 98 | 80 | 100 | 100 |
| 2385.45 | 45.47 | 98 | 80 | 81 | 94 |
| 2046.99 | 45.39 | 98 | 50 | 56 | 50 |
| 2409.90 | 32.54 | 98 | 100 | 94 | 94 |
| 3433.28 | 44.46 | 98 | 70 | 56 | 56 |
| 1545.75 | 54.72 | 98 | 90 | 100 | 89 |
| 2736.31 | 32.30 | 97 | 100 | 94 | 89 |
| 3723.33 | 32.48 | 97 | 20 | 50 | 22 |
| 1737.76 | 41.29 | 97 | 90 | 88 | 89 |
| 1378.54 | 45.45 | 97 | 60 | 81 | 56 |
| 2854.41 | 43.80 | 97 | 50 | 75 | 56 |
| 2068.54 | 41.06 | 97 | 80 | 81 | 61 |
| 2663.36 | 36.38 | 97 | 80 | 88 | 83 |
| 2085.50 | 39.21 | 97 | 100 | 94 | 94 |
| 2682.49 | 35.03 | 97 | 90 | 75 | 89 |
| 1046.57 | 38.63 | 97 | 20 | 38 | 6 |
| 2994.61 | 40.63 | 97 | 90 | 88 | 72 |
| 2583.98 | 43.75 | 97 | 30 | 62 | 28 |
| 2129.48 | 35.14 | 97 | 60 | 50 | 17 |
| 981.56 | 37.39 | 97 | 50 | 81 | 56 |
| 879.55 | 26.95 | 97 | 60 | 94 | 22 |
| 2394.29 | 36.32 | 97 | 100 | 100 | 94 |
| 3986.98 | 30.46 | 97 | 50 | 69 | 44 |
| 2483.58 | 38.74 | 97 | 90 | 81 | 78 |
| 1523.73 | 54.29 | 97 | 90 | 100 | 89 |
| 1889.76 | 46.38 | 97 | 30 | 44 | 39 |
| 1507.64 | 54.43 | 97 | 90 | 100 | 89 |
| 3209.22 | 34.27 | 97 | 30 | 81 | 33 |
| 3022.82 | 33.82 | 97 | 40 | 62 | 61 |
| 1765.17 | 35.50 | 97 | 90 | 88 | 94 |
| 1367.67 | 53.27 | 97 | 90 | 94 | 94 |
| 2726.38 | 40.38 | 97 | 100 | 88 | 100 |
| 1179.57 | 52.17 | 97 | 80 | 100 | 83 |
| 1651.86 | 55.15 | 97 | 90 | 100 | 78 |
| 3376.24 | 45.17 | 97 | 50 | 88 | 67 |
| 3293.15 | 54.21 | 97 | 50 | 75 | 50 |
| 1579.50 | 39.43 | 97 | 100 | 94 | 94 |
| 3474.27 | 43.37 | 95 | 70 | 75 | 67 |
| 2848.83 | 36.33 | 95 | 70 | 75 | 44 |
| 3319.28 | 46.22 | 95 | 50 | 62 | 39 |
| 1000.52 | 33.96 | 95 | 40 | 44 | 28 |
| 3281.97 | 49.44 | 95 | 60 | 50 | 56 |
| 1885.74 | 57.47 | 95 | 70 | 75 | 28 |
| 3556.92 | 34.85 | 95 | 100 | 75 | 78 |
| 1609.17 | 42.60 | 95 | 100 | 94 | 89 |
| 2767.41 | 31.39 | 95 | 40 | 69 | 56 |
| 3108.81 | 44.70 | 95 | 50 | 75 | 56 |
| 2233.00 | 31.06 | 95 | 30 | 69 | 56 |
| 882.55 | 36.55 | 95 | 60 | 31 | 72 |
| 1680.16 | 37.32 | 95 | 100 | 94 | 100 |
| 1673.80 | 54.59 | 95 | 80 | 94 | 61 |
| 2336.78 | 42.47 | 95 | 70 | 94 | 89 |
| 1217.64 | 48.54 | 95 | 100 | 94 | 83 |
| 1489.49 | 42.21 | 95 | 90 | 94 | 72 |
| 2442.06 | 46.85 | 95 | 70 | 81 | 67 |
| 2279.06 | 47.16 | 95 | 70 | 69 | 44 |
| 4748.51 | 25.38 | 95 | 50 | 38 | 33 |
| 1766.84 | 35.15 | 95 | 100 | 100 | 100 |

TABLE 15

| | | Frequency | Frequency | Frequency | Frequency |
|---|---|---|---|---|---|
| Mass | CE-time | Healthy | FSGS | MCD | MGN |
| [Da] | [min] | [%] | [%] | [%] | [%] |
| 1435.69 | 32.7 | 94 | 86 | 100 | 7 |
| 1282.39 | 29.3 | 69 | 29 | 29 | 0 |
| 3531.01 | 26.9 | 69 | 0 | 0 | 0 |
| 5801.94 | 13.3 | 69 | 0 | 7 | 7 |

TABLE 16
healthy vs. renal patients

| mass [Da] | CE time [min] |
|---|---|
| 909.4 | 40.3 |
| 1159.6 | 39.0 |
| 1338.7 | 47.2 |
| 1686.8 | 38.2 |
| 1847.8 | 57.0 |
| 1966.3 | 25.1 |
| 1990.8 | 47.3 |
| 2146.3 | 25.8 |
| 2432.2 | 38.3 |
| 2465.0 | 22.8 |
| 3707.0 | 31.9 |

TABLE 17
MGN vs. MCD

| Mass | CE_t |
|---|---|
| 879.6 | 26.9 |
| 1279.7 | 38.3 |
| 1341.8 | 33.1 |
| 1404.9 | 29.4 |
| 1569.8 | 48.3 |
| 1574.8 | 33.9 |
| 1605.9 | 23.7 |
| 2527.3 | 40.8 |
| 5112.9 | 33.1 |

TABLE 18
MCD vs. FSGS

| Mass | CE_t |
|---|---|
| 1199.6 | 31.0 |
| 1826.9 | 50.8 |
| 2077.3 | 35.8 |
| 2258.9 | 33.6 |
| 2918.0 | 42.2 |

TABLE 19
MGN vs. FSGS

| Mass | CE_t |
|---|---|
| 2312.5 | 22.9 |
| 2453.6 | 32.0 |
| 2639.6 | 45.2 |
| 9182.0 | 17.1 |

TABLE 20

| mass | CE time | % | % | mass | CE time | % | % |
|---|---|---|---|---|---|---|---|
| [Da] | [min] | healthy | MGN | [Da] | [min] | healthy | MGN |
| 4098.2 | 40.1 | 100 | 0 | 1933.02 | 41.5 | 100 | 12 |
| 3685.9 | 35.9 | 100 | 0 | 1889.82 | 49.3 | 100 | 12 |
| 3531.3 | 42.9 | 100 | 0 | 1636.69 | 46.8 | 100 | 12 |
| 3359.7 | 48.4 | 100 | 0 | 1579.76 | 47.3 | 100 | 12 |
| 3287.4 | 47.4 | 100 | 0 | 1438.66 | 45.4 | 100 | 12 |
| 3265.3 | 51.6 | 100 | 0 | 1321.59 | 45.8 | 100 | 12 |
| 3098.5 | 46.9 | 100 | 0 | 1255.53 | 53 | 100 | 12 |
| 3041.3 | 46.5 | 100 | 0 | 1200.53 | 53.4 | 100 | 12 |
| 3011.3 | 46.5 | 100 | 0 | 2427.43 | 27 | 12 | 100 |
| 2742.2 | 45.8 | 100 | 0 | 1829.09 | 33.8 | 12 | 100 |
| 2563.2 | 34.5 | 100 | 0 | 4627.01 | 28.7 | 0 | 88 |
| 2483.5 | 44.5 | 100 | 0 | 2621.42 | 29 | 0 | 88 |
| 2385.2 | 50.4 | 100 | 0 | 1942.57 | 34.8 | 0 | 88 |
| 1893.1 | 42.5 | 100 | 0 | 1867.06 | 33.8 | 0 | 88 |
| 1639.9 | 47.5 | 100 | 0 | 1759.92 | 32.5 | 0 | 88 |
| 1609.7 | 47.3 | 100 | 0 | 1460.83 | 39.8 | 0 | 88 |
| 1580.9 | 41.3 | 100 | 0 | 3013.36 | 36.6 | 88 | 12 |
| 1508.7 | 46.6 | 100 | 0 | 2838.9 | 39.8 | 88 | 12 |
| 1489.6 | 46.2 | 100 | 0 | 2710.31 | 52.8 | 88 | 12 |
| 1424.7 | 56 | 100 | 0 | 2395.04 | 40.4 | 88 | 12 |
| 1407.6 | 54.6 | 100 | 0 | 1876.91 | 37.2 | 88 | 12 |
| 1160.6 | 52.7 | 100 | 0 | 1863.86 | 59.2 | 88 | 12 |
| 981.53 | 41 | 100 | 0 | 1651.81 | 56.5 | 88 | 12 |
| 980.54 | 38 | 100 | 0 | 1561.56 | 56.1 | 88 | 12 |
| 876.4 | 52.2 | 100 | 0 | 1523.72 | 56.1 | 88 | 12 |
| 2752.9 | 29.3 | 0 | 100 | 1473.66 | 46.4 | 88 | 12 |
| 6171.1 | 43.4 | 88 | 0 | 1261.49 | 53.2 | 88 | 12 |
| 3851.9 | 41.2 | 88 | 0 | 1195.5 | 54 | 88 | 12 |
| 3706.8 | 35.2 | 88 | 0 | 10047 | 22.3 | 12 | 88 |
| 3634.2 | 43.6 | 88 | 0 | 4713.94 | 28.8 | 12 | 88 |
| 3631.3 | 36.3 | 88 | 0 | 4241.41 | 26.7 | 12 | 88 |
| 3478.9 | 47.9 | 88 | 0 | 1811.13 | 34.6 | 12 | 88 |
| 3376.3 | 48.5 | 88 | 0 | 1753.98 | 32.7 | 12 | 88 |
| 3338.2 | 38.6 | 88 | 0 | 1698.06 | 34.1 | 12 | 88 |
| 3292.7 | 56.7 | 88 | 0 | 1584.91 | 32.7 | 12 | 88 |
| 3280.6 | 42.2 | 88 | 0 | 4353.62 | 33.6 | 75 | 0 |
| 3271.5 | 47.3 | 88 | 0 | 4102.45 | 45.2 | 75 | 0 |
| 3248.5 | 47.2 | 88 | 0 | 4044.58 | 34.1 | 75 | 0 |
| 2849.2 | 39.4 | 88 | 0 | 3987.48 | 34.8 | 75 | 0 |
| 2736.4 | 39.3 | 88 | 0 | 3947.22 | 36 | 75 | 0 |
| 2682.1 | 37.3 | 88 | 0 | 3589.65 | 41.3 | 75 | 0 |
| 2642.6 | 44.7 | 88 | 0 | 3433.12 | 48.6 | 75 | 0 |
| 2584.3 | 51.9 | 88 | 0 | 3416.92 | 48.6 | 75 | 0 |
| 2257.1 | 50.3 | 88 | 0 | 3295.53 | 42 | 75 | 0 |
| 2204.9 | 44 | 88 | 0 | 3261.55 | 35.6 | 75 | 0 |
| 2196.9 | 49.9 | 88 | 0 | 3258.52 | 37.8 | 75 | 0 |
| 2039.2 | 35.8 | 88 | 0 | 3193.48 | 37.3 | 75 | 0 |
| 1680.8 | 47 | 88 | 0 | 3152.6 | 40.3 | 75 | 0 |
| 1635.8 | 56.8 | 88 | 0 | 3092.08 | 47.6 | 75 | 0 |
| 1539.7 | 46.3 | 88 | 0 | 2863.25 | 40.6 | 75 | 0 |
| 1423.7 | 54.4 | 88 | 0 | 2854.55 | 52.4 | 75 | 0 |
| 1422.5 | 55 | 88 | 0 | 2698.37 | 37.2 | 75 | 0 |
| 1353.7 | 43.2 | 88 | 0 | 2548.42 | 37.7 | 75 | 0 |
| 1046.6 | 42.6 | 88 | 0 | 2464.07 | 50.8 | 75 | 0 |
| 3969.5 | 34.4 | 100 | 12 | 2406.98 | 50.6 | 75 | 0 |
| 3496.1 | 47.1 | 100 | 12 | 2279 | 50.2 | 75 | 0 |
| 3442.2 | 47.9 | 100 | 12 | 2233.02 | 35.7 | 75 | 0 |
| 3405.4 | 42.4 | 100 | 12 | 2226.97 | 43 | 75 | 0 |
| 3385.6 | 41.5 | 100 | 12 | 2019.97 | 41.1 | 75 | 0 |
| 3281.7 | 53 | 100 | 12 | 1991.95 | 36.2 | 75 | 0 |
| 3209.4 | 37.1 | 100 | 12 | 1849.85 | 41.1 | 75 | 0 |
| 2799.9 | 42.4 | 100 | 12 | 1768.95 | 48.7 | 75 | 0 |
| 2378 | 38.8 | 100 | 12 | 1755.02 | 48.2 | 75 | 0 |
| 2170 | 42.6 | 100 | 12 | 1737.78 | 48.2 | 75 | 0 |
| 2008 | 37 | 100 | 12 | 1462.63 | 56.1 | 75 | 0 |
| 1949 | 41.5 | 100 | 12 | 1446.6 | 56 | 75 | 0 |
| | | | | 1425.78 | 41.6 | 75 | 0 |

TABLE 21

| mass | CE time | | |
|---|---|---|---|
| [Da] | [min] | % healthy | % MGN |
| 1405.6 | 55.7 | 75 | 0 |
| 1389.6 | 55.3 | 75 | 0 |
| 1322.6 | 45.4 | 75 | 0 |
| 1262.6 | 56.4 | 75 | 0 |
| 1246.6 | 55.6 | 75 | 0 |
| 1224.8 | 35.4 | 75 | 0 |
| 1141.7 | 41.6 | 75 | 0 |
| 1028.6 | 41.9 | 75 | 0 |
| 946.43 | 50.5 | 75 | 0 |
| 3723.1 | 35.5 | 100 | 25 |
| 3458.2 | 48.2 | 100 | 25 |
| 3001.8 | 51.8 | 100 | 25 |
| 2825.3 | 40.8 | 100 | 25 |
| 2695.3 | 39.1 | 100 | 25 |
| 2679.2 | 39.2 | 100 | 25 |
| 2410 | 39.6 | 100 | 25 |
| 2394 | 39.3 | 100 | 25 |
| 2048 | 35.9 | 100 | 25 |
| 1911.1 | 41.6 | 100 | 25 |
| 1545.7 | 57.3 | 100 | 25 |
| 1507.7 | 57.3 | 100 | 25 |
| 1467.8 | 41 | 100 | 25 |
| 1451.7 | 46.4 | 100 | 25 |
| 1435.7 | 46.3 | 100 | 25 |
| 1265.6 | 44.6 | 100 | 25 |
| 1250.6 | 45.7 | 100 | 25 |
| 1239.4 | 53.7 | 100 | 25 |
| 1235.6 | 44 | 100 | 25 |
| 1217.6 | 53.3 | 100 | 25 |
| 1194.6 | 44.1 | 100 | 25 |
| 1179.5 | 55 | 100 | 25 |
| 1716 | 32.1 | 25 | 100 |
| 4827.2 | 29.3 | 0 | 75 |
| 2937.4 | 29.6 | 0 | 75 |
| 2057.4 | 37 | 0 | 75 |
| 1851.1 | 33.8 | 0 | 75 |
| 1680.1 | 33.6 | 0 | 75 |
| 1517.9 | 30.2 | 0 | 75 |
| 1483.9 | 32.5 | 0 | 75 |
| 1481.9 | 33.8 | 0 | 75 |
| 1404.8 | 29 | 0 | 75 |
| 1398.8 | 34.1 | 0 | 75 |
| 1367.6 | 56.1 | 88 | 25 |
| 1157.6 | 54.9 | 88 | 25 |
| 3474.3 | 47.9 | 75 | 12 |
| 3402.5 | 37 | 75 | 12 |
| 2761.4 | 34.7 | 75 | 12 |
| 2644.1 | 33.5 | 75 | 12 |
| 2587.2 | 34.9 | 75 | 12 |
| 2579.7 | 50.5 | 75 | 12 |
| 2579.7 | 41.4 | 75 | 12 |
| 2175 | 50 | 75 | 12 |
| 2069.1 | 49.5 | 75 | 12 |
| 2047 | 49.5 | 75 | 12 |
| 1170.6 | 46 | 75 | 12 |
| 1386.8 | 32.3 | 25 | 88 |
| 8766.7 | 21.6 | 12 | 75 |
| 4154.4 | 26.4 | 12 | 75 |
| 3842.8 | 25.7 | 12 | 75 |
| 1873 | 33.9 | 12 | 75 |
| 1566.9 | 33.2 | 12 | 75 |
| 1499.9 | 33.7 | 12 | 75 |
| 1368.8 | 31.6 | 12 | 75 |
| 1285.7 | 31.1 | 12 | 75 |
| 1108.6 | 32.2 | 12 | 75 |
| 1099.6 | 31.2 | 12 | 75 |
| 1060.6 | 31.6 | 12 | 75 |

TABLE 22

| | | fre- | | |
|---|---|---|---|---|
| | | healthy, | disease, | |
| time [min] | mass [Da] | quency | frequency | type |
| 22.9 ± 3.05 | 834.5 ± 0.10 | 3% | 54% | Diabetes pos. |
| 22.9 ± 3.03 | 869.4 ± 0.17 | 14% | 63% | Diabetes pos. |
| 24.2 ± 1.89 | 874.5 ± 0.09 | 28% | 66% | Diabetes pos. |
| 22.2 ± 2.19 | 907.5 ± 0.13 | 0% | 41% | Diabetes pos. |
| 29.0 ± 2.35 | 910.5 ± 0.09 | 15% | 47% | Diabetes pos. |
| 22.9 ± 3.18 | 947.6 ± 0.22 | 17% | 51% | Diabetes pos. |
| 26.8 ± 2.98 | 950.5 ± 0.12 | 0% | 24% | Diabetes pos. |
| 23.2 ± 4.87 | 995.6 ± 0.14 | 23% | 50% | Diabetes pos. |
| 27.4 ± 3.59 | 1082.6 ± 0.16 | 0% | 44% | Diabetes pos. |
| 32.3 ± 1.99 | 1096.5 ± 0.14 | 10% | 51% | Diabetes pos. |
| 26.8 ± 3.85 | 1176.6 ± 0.13 | 21% | 59% | Diabetes pos. |
| 22.3 ± 3.45 | 1222.8 ± 0.22 | 17% | 56% | Diabetes pos. |
| 30.6 ± 3.31 | 1236.6 ± 0.11 | 24% | 59% | Diabetes pos. |
| 52.6 ± 4.80 | 1285.0 ± 0.09 | 14% | 54% | Diabetes pos. |
| 28.8 ± 3.98 | 1332.7 ± 0.20 | 23% | 55% | Diabetes pos. |
| 49.8 ± 4.72 | 1332.8 ± 0.16 | 8% | 38% | Diabetes pos. |
| 26.7 ± 2.79 | 1355.8 ± 0.15 | 17% | 56% | Diabetes pos. |
| 24.6 ± 2.84 | 1386.8 ± 0.14 | 53% | 77% | Diabetes pos. |
| 26.8 ± 3.26 | 1403.7 ± 0.21 | 8% | 46% | Diabetes pos. |
| 17.8 ± 4.12 | 1405.9 ± 0.15 | 14% | 56% | Diabetes pos. |
| 31.5 ± 3.71 | 1442.7 ± 0.27 | 15% | 55% | Diabetes pos. |
| 32.1 ± 3.38 | 1449.8 ± 0.14 | 41% | 85% | Diabetes pos. |
| 31.3 ± 5.27 | 1592.4 ± 0.38 | 3% | 46% | Diabetes pos. |
| 43.4 ± 4.41 | 1783.4 ± 0.30 | 33% | 63% | Diabetes pos. |
| 29.4 ± 3.08 | 1789.2 ± 0.39 | 28% | 75% | Diabetes pos. |
| 38.4 ± 1.09 | 1818.9 ± 0.21 | 28% | 67% | Diabetes pos. |
| 37.7 ± 1.04 | 1821.4 ± 0.39 | 14% | 56% | Diabetes pos. |
| 24.4 ± 2.55 | 1829.2 ± 0.23 | 45% | 81% | Diabetes pos. |
| 51.1 ± 4.11 | 1854.7 ± 0.41 | 14% | 54% | Diabetes pos. |
| 37.6 ± 3.30 | 1856.8 ± 0.48 | 33% | 56% | Diabetes pos. |
| 24.7 ± 2.63 | 1872.9 ± 0.35 | 43% | 72% | Diabetes pos. |
| 28.3 ± 3.47 | 1949.5 ± 0.32 | 17% | 73% | Diabetes pos. |
| 31.6 ± 2.90 | 1955.1 ± 0.32 | 55% | 79% | Diabetes pos. |
| 31.3 ± 3.00 | 1971.0 ± 0.45 | 20% | 54% | Diabetes pos. |
| 37.8 ± 2.40 | 2032.0 ± 0.30 | 25% | 60% | Diabetes pos. |
| 30.9 ± 4.69 | 2061.4 ± 0.58 | 10% | 38% | Diabetes pos. |
| 33.8 ± 3.76 | 2092.2 ± 0.46 | 18% | 45% | Diabetes pos. |
| 27.7 ± 4.43 | 2185.6 ± 0.46 | 10% | 36% | Diabetes pos. |
| 32.9 ± 1.48 | 2189.4 ± 0.34 | 14% | 54% | Diabetes pos. |
| 39.6 ± 5.31 | 2229.4 ± 0.48 | 5% | 39% | Diabetes pos. |
| 24.5 ± 5.14 | 2229.9 ± 0.33 | 25% | 63% | Diabetes pos. |
| 28.3 ± 3.30 | 2502.9 ± 0.56 | 20% | 48% | Diabetes pos. |
| 24.9 ± 4.84 | 2621.6 ± 0.97 | 20% | 45% | Diabetes pos. |
| 37.5 ± 4.52 | 2669.8 ± 0.39 | 23% | 67% | Diabetes pos. |
| 20.8 ± 4.47 | 2752.2 ± 0.76 | 35% | 64% | Diabetes pos. |
| 24.9 ± 4.31 | 2795.7 ± 0.96 | 13% | 40% | Diabetes pos. |
| 48.2 ± 3.61 | 3246.1 ± 0.43 | 0% | 30% | Diabetes pos. |
| 20.9 ± 3.33 | 3844.0 ± 0.52 | 3% | 54% | Diabetes pos. |
| 21.9 ± 2.62 | 4961.5 ± 0.89 | 10% | 40% | Diabetes pos. |
| 18.6 ± 2.91 | 5497.0 ± 0.66 | 18% | 42% | Diabetes pos. |
| 20.4 ± 2.20 | 808.4 ± 0.10 | 58% | 9% | Diabetes neg. |
| 45.3 ± 2.03 | 897.5 ± 0.09 | 48% | 7% | Diabetes neg. |
| 31.4 ± 1.08 | 929.5 ± 0.11 | 98% | 46% | Diabetes neg. |
| 41.2 ± 1.41 | 946.4 ± 0.10 | 85% | 36% | Diabetes neg. |
| 28.0 ± 1.04 | 980.5 ± 0.07 | 85% | 31% | Diabetes neg. |
| 26.7 ± 2.26 | 1000.5 ± 0.09 | 83% | 41% | Diabetes neg. |
| 27.8 ± 1.51 | 1008.5 ± 0.10 | 95% | 41% | Diabetes neg. |
| 29.3 ± 2.55 | 1012.5 ± 0.10 | 63% | 17% | Diabetes neg. |
| 43.6 ± 2.03 | 1047.5 ± 0.11 | 90% | 26% | Diabetes neg. |
| 25.0 ± 3.91 | 1052.6 ± 0.08 | 45% | 4% | Diabetes neg. |
| 37.4 ± 5.63 | 1066.5 ± 0.14 | 58% | 13% | Diabetes neg. |
| 22.8 ± 1.78 | 1075.5 ± 0.13 | 68% | 26% | Diabetes neg. |
| 28.9 ± 3.89 | 1088.6 ± 0.15 | 65% | 21% | Diabetes neg. |
| 44.4 ± 2.06 | 1106.5 ± 0.11 | 80% | 18% | Diabetes neg. |
| 34.1 ± 1.80 | 1107.5 ± 0.10 | 88% | 35% | Diabetes neg. |
| 42.8 ± 3.26 | 1120.5 ± 0.06 | 60% | 14% | Diabetes neg. |
| 29.1 ± 2.26 | 1134.6 ± 0.10 | 95% | 49% | Diabetes neg. |
| 28.2 ± 3.00 | 1137.7 ± 0.11 | 70% | 24% | Diabetes neg. |
| 45.5 ± 2.34 | 1139.5 ± 0.20 | 83% | 22% | Diabetes neg. |
| 32.9 ± 1.25 | 1159.6 ± 0.11 | 80% | 27% | Diabetes neg. |
| 23.3 ± 4.17 | 1180.5 ± 0.16 | 50% | 9% | Diabetes neg. |
| 43.8 ± 2.08 | 1200.6 ± 0.11 | 95% | 50% | Diabetes neg. |
| 27.2 ± 3.22 | 1204.6 ± 0.17 | 60% | 17% | Diabetes neg. |
| 44.9 ± 2.53 | 1209.5 ± 0.09 | 83% | 17% | Diabetes neg. |
| 47.8 ± 2.73 | 1224.6 ± 0.12 | 75% | 19% | Diabetes neg. |
| 25.6 ± 2.43 | 1246.7 ± 0.15 | 73% | 30% | Diabetes neg. |
| 47.9 ± 2.66 | 1268.6 ± 0.09 | 68% | 25% | Diabetes neg. |
| 43.9 ± 1.80 | 1277.5 ± 0.10 | 70% | 28% | Diabetes neg. |
| 46.0 ± 2.69 | 1278.5 ± 0.09 | 58% | 10% | Diabetes neg. |
| 33.1 ± 1.82 | 1282.6 ± 0.13 | 62% | 7% | Diabetes neg. |
| 29.3 ± 3.88 | 1331.7 ± 0.18 | 65% | 12% | Diabetes neg. |
| 45.9 ± 4.78 | 1405.5 ± 0.33 | 93% | 45% | Diabetes neg. |
| 44.4 ± 3.90 | 1423.6 ± 0.16 | 60% | 20% | Diabetes neg. |
| 19.2 ± 3.40 | 1484.8 ± 0.19 | 68% | 13% | Diabetes neg. |
| 36.9 ± 2.02 | 1609.6 ± 0.13 | 85% | 13% | Diabetes neg. |
| 38.9 ± 3.78 | 1639.7 ± 0.27 | 63% | 19% | Diabetes neg. |
| 33.2 ± 3.34 | 1662.9 ± 0.21 | 62% | 5% | Diabetes neg. |
| 35.8 ± 2.19 | 1684.6 ± 0.29 | 66% | 10% | Diabetes neg. |
| 36.2 ± 4.78 | 1666.6 ± 0.34 | 75% | 29% | Diabetes neg. |
| 35.9 ± 2.98 | 1678.1 ± 0.44 | 60% | 18% | Diabetes neg. |
| 37.3 ± 2.99 | 1716.8 ± 0.23 | 73% | 19% | Diabetes neg. |
| 46.5 ± 4.38 | 1717.5 ± 0.37 | 79% | 15% | Diabetes neg. |
| 37.9 ± 4.18 | 1746.0 ± 0.33 | 83% | 34% | Diabetes neg. |
| 25.1 ± 2.25 | 1817.6 ± 0.27 | 65% | 8% | Diabetes neg. |
| 34.2 ± 3.95 | 1823.4 ± 0.47 | 73% | 30% | Diabetes neg. |
| 29.1 ± 3.59 | 1849.8 ± 0.30 | 100% | 56% | Diabetes neg. |
| 49.3 ± 4.49 | 1914.1 ± 0.36 | 88% | 38% | Diabetes neg. |
| 44.2 ± 4.23 | 1916.7 ± 0.33 | 69% | 10% | Diabetes neg. |
| 39.8 ± 2.19 | 2030.8 ± 0.35 | 93% | 38% | Diabetes neg. |
| 31.9 ± 1.61 | 2118.9 ± 0.21 | 73% | 14% | Diabetes neg. |
| 41.2 ± 2.45 | 2179.3 ± 0.42 | 58% | 17% | Diabetes neg. |
| 20.1 ± 2.78 | 2219.0 ± 0.26 | 53% | 13% | Diabetes neg. |
| 25.8 ± 2.70 | 2256.9 ± 0.47 | 85% | 26% | Diabetes neg. |
| 45.1 ± 5.23 | 2273.4 ± 0.42 | 79% | 22% | Diabetes neg. |
| 40.7 ± 1.90 | 2279.0 ± 0.33 | 90% | 20% | Diabetes neg. |
| 26.8 ± 3.73 | 2320.2 ± 0.55 | 78% | 34% | Diabetes neg. |
| 23.6 ± 3.10 | 2332.2 ± 0.35 | 53% | 11% | Diabetes neg. |
| 44.5 ± 3.08 | 2345.6 ± 0.46 | 75% | 34% | Diabetes neg. |
| 25.7 ± 5.16 | 2384.5 ± 0.63 | 65% | 21% | Diabetes neg. |
| 38.5 ± 3.62 | 2423.9 ± 0.41 | 88% | 29% | Diabetes neg. |
| 34.2 ± 2.92 | 2429.9 ± 0.51 | 65% | 18% | Diabetes neg. |
| 23.3 ± 2.54 | 2443.3 ± 0.46 | 66% | 5% | Diabetes neg. |
| 41.7 ± 3.72 | 2548.1 ± 0.57 | 69% | 15% | Diabetes neg. |
| 27.3 ± 4.77 | 2548.3 ± 0.66 | 83% | 35% | Diabetes neg. |
| 43.6 ± 2.08 | 2548.3 ± 0.23 | 95% | 41% | Diabetes neg. |
| 24.0 ± 3.11 | 2581.5 ± 0.47 | 60% | 13% | Diabetes neg. |
| 24.0 ± 2.70 | 2587.4 ± 0.40 | 80% | 26% | Diabetes neg. |
| 41.7 ± 3.06 | 2606.8 ± 0.55 | 78% | 35% | Diabetes neg. |
| 31.3 ± 4.92 | 2636.4 ± 0.48 | 72% | 12% | Diabetes neg. |
| 25.5 ± 3.62 | 2644.2 ± 0.41 | 88% | 33% | Diabetes neg. |
| 29.2 ± 1.07 | 2654.0 ± 0.37 | 66% | 0% | Diabetes neg. |
| 29.8 ± 3.50 | 2698.2 ± 0.63 | 90% | 29% | Diabetes neg. |
| 43.0 ± 2.26 | 2710.5 ± 0.37 | 79% | 5% | Diabetes neg. |
| 25.1 ± 1.64 | 2761.3 ± 0.35 | 88% | 44% | Diabetes neg. |
| 31.3 ± 2.79 | 2808.5 ± 0.56 | 79% | 22% | Diabetes neg. |
| 42.0 ± 3.22 | 2876.5 ± 0.48 | 62% | 7% | Diabetes neg. |
| 33.7 ± 3.34 | 2898.7 ± 0.50 | 85% | 43% | Diabetes neg. |
| 42.2 ± 2.68 | 2908.1 ± 0.53 | 72% | 17% | Diabetes neg. |
| 35.4 ± 2.63 | 2917.6 ± 0.58 | 72% | 12% | Diabetes neg. |
| 35.4 ± 0.77 | 2978.1 ± 0.49 | 85% | 35% | Diabetes neg. |
| 36.1 ± 1.42 | 2994.6 ± 0.80 | 83% | 24% | Diabetes neg. |
| 43.5 ± 2.99 | 3023.4 ± 0.65 | 93% | 34% | Diabetes neg. |
| 44.4 ± 3.35 | 3045.2 ± 0.61 | 69% | 12% | Diabetes neg. |
| 22.9 ± 3.47 | 3076.4 ± 0.96 | 66% | 7% | Diabetes neg. |
| 35.7 ± 1.99 | 3082.3 ± 0.43 | 73% | 22% | Diabetes neg. |
| 33.6 ± 3.53 | 3136.8 ± 0.61 | 95% | 47% | Diabetes neg. |
| 21.7 ± 3.14 | 3154.8 ± 0.44 | 55% | 10% | Diabetes neg. |
| 26.5 ± 1.92 | 3193.7 ± 0.53 | 78% | 32% | Diabetes neg. |
| 24.4 ± 3.02 | 3206.3 ± 0.72 | 66% | 7% | Diabetes neg. |
| 28.2 ± 2.80 | 3250.9 ± 0.71 | 63% | 18% | Diabetes neg. |
| 48.2 ± 3.46 | 3293.2 ± 0.74 | 93% | 39% | Diabetes neg. |
| 31.4 ± 1.60 | 3295.7 ± 0.33 | 95% | 40% | Diabetes neg. |
| 27.2 ± 3.58 | 3338.4 ± 0.79 | 80% | 34% | Diabetes neg. |
| 37.3 ± 2.11 | 3381.6 ± 0.63 | 78% | 26% | Diabetes neg. |
| 27.6 ± 2.49 | 3452.1 ± 0.49 | 58% | 15% | Diabetes neg. |
| 37.3 ± 1.50 | 3463.0 ± 0.83 | 72% | 15% | Diabetes neg. |
| 19.6 ± 2.89 | 3583.4 ± 0.75 | 79% | 20% | Diabetes neg. |
| 34.0 ± 2.55 | 3634.4 ± 0.74 | 86% | 29% | Diabetes neg. |
| 37.7 ± 2.61 | 3681.8 ± 1.38 | 55% | 14% | Diabetes neg. |
| 25.5 ± 2.25 | 3686.2 ± 0.60 | 86% | 20% | Diabetes neg. |
| 36.0 ± 3.89 | 3735.7 ± 0.57 | 70% | 28% | Diabetes neg. |
| 30.3 ± 1.58 | 3852.3 ± 0.56 | 83% | 41% | Diabetes neg. |
| 29.6 ± 1.46 | 4098.4 ± 0.59 | 93% | 20% | Diabetes neg. |
| 28.8 ± 1.18 | 5428.8 ± 0.67 | 70% | 19% | Diabetes neg. |
| 33.1 ± 0.69 | 6187.5 ± 1.13 | 83% | 10% | Diabetes neg. |
| 26.0 ± 4.82 | 6212.0 ± 1.41 | 75% | 26% | Diabetes neg. |
| 23.3 ± 2.19 | 9868.8 ± 1.33 | 66% | 0% | Diabetes neg. |
| 21.7 ± 5.12 | 830.5 ± 0.11 | 4% | 40% | Nephropathy pos. |
| 32.4 ± 1.83 | 866.4 ± 0.11 | 0% | 40% | Nephropathy pos. |
| 30.6 ± 3.07 | 909.5 ± 0.13 | 11% | 40% | Nephropathy pos. |
| 32.8 ± 3.14 | 937.5 ± 0.11 | 14% | 73% | Nephropathy pos. |
| 24.9 ± 2.97 | 952.5 ± 0.16 | 11% | 40% | Nephropathy pos. |
| 32.1 ± 2.44 | 1033.5 ± 0.11 | 5% | 40% | Nephropathy pos. |
| 24.4 ± 2.87 | 1060.6 ± 0.16 | 17% | 68% | Nephropathy pos. |
| 27.5 ± 2.86 | 1131.6 ± 0.16 | 20% | 68% | Nephropathy pos. |
| 33.4 ± 3.48 | 1181.6 ± 0.15 | 22% | 73% | Nephropathy pos. |
| 33.0 ± 2.52 | 1203.6 ± 0.14 | 9% | 50% | Nephropathy pos. |
| 26.5 ± 3.68 | 1211.6 ± 0.14 | 14% | 40% | Nephropathy pos. |
| 33.1 ± 0.91 | 1219.6 ± 0.15 | 18% | 40% | Nephropathy pos. |
| 32.8 ± 3.30 | 1225.6 ± 0.13 | 12% | 40% | Nephropathy pos. |
| 30.7 ± 3.18 | 1297.7 ± 0.20 | 31% | 82% | Nephropathy pos. |
| 34.1 ± 2.05 | 1333.7 ± 0.23 | 9% | 40% | Nephropathy pos. |
| 44.7 ± 4.06 | 1337.5 ± 0.20 | 19% | 59% | Nephropathy pos. |
| 27.9 ± 4.19 | 1398.8 ± 0.36 | 29% | 77% | Nephropathy pos. |
| 21.3 ± 5.08 | 1423.7 ± 0.49 | 6% | 50% | Nephropathy pos. |
| 28.1 ± 4.95 | 1439.8 ± 0.19 | 19% | 68% | Nephropathy pos. |
| 24.5 ± 2.42 | 1466.0 ± 0.27 | 9% | 77% | Nephropathy pos. |
| 27.5 ± 4.93 | 1482.0 ± 0.42 | 33% | 40% | Nephropathy pos. |
| 29.8 ± 4.43 | 1482.9 ± 0.28 | 18% | 40% | Nephropathy pos. |
| 24.3 ± 2.65 | 1483.7 ± 0.28 | 26% | 91% | Nephropathy pos. |
| 24.6 ± 1.98 | 1500.0 ± 0.20 | 38% | 86% | Nephropathy pos. |
| 24.6 ± 2.90 | 1553.1 ± 0.28 | 14% | 64% | Nephropathy pos. |
| 29.0 ± 4.83 | 1556.7 ± 0.45 | 26% | 73% | Nephropathy pos. |
| 24.2 ± 2.48 | 1567.0 ± 0.22 | 26% | 86% | Nephropathy pos. |
| 28.8 ± 4.53 | 1596.9 ± 0.31 | 21% | 86% | Nephropathy pos. |
| 24.5 ± 2.43 | 1652.8 ± 0.25 | 14% | 59% | Nephropathy pos. |
| 26.3 ± 2.63 | 1669.8 ± 0.37 | 20% | 64% | Nephropathy pos. |
| 33.1 ± 3.22 | 1729.2 ± 0.36 | 6% | 45% | Nephropathy pos. |
| 30.5 ± 4.11 | 1744.4 ± 0.46 | 16% | 59% | Nephropathy pos. |
| 25.1 ± 3.42 | 1754.4 ± 0.41 | 53% | 95% | Nephropathy pos. |
| 24.2 ± 1.56 | 1776.0 ± 0.27 | 9% | 50% | Nephropathy pos. |
| 18.5 ± 3.55 | 1791.0 ± 0.38 | 7% | 40% | Nephropathy pos. |
| 32.2 ± 5.38 | 1792.9 ± 0.31 | 28% | 40% | Nephropathy pos. |
| 9.7 ± 2.54 | 1799.8 ± 0.29 | 0% | 40% | Nephropathy pos. |
| 25.3 ± 2.89 | 1810.9 ± 0.38 | 43% | 91% | Nephropathy pos. |
| 24.6 ± 2.34 | 1851.1 ± 0.21 | 43% | 95% | Nephropathy pos. |
| 27.2 ± 4.46 | 1867.3 ± 0.42 | 38% | 91% | Nephropathy pos. |
| 25.0 ± 3.97 | 1966.0 ± 0.53 | 16% | 40% | Nephropathy pos. |
| 28.7 ± 3.08 | 1982.8 ± 0.57 | 11% | 40% | Nephropathy pos. |
| 29.5 ± 5.53 | 1986.3 ± 0.36 | 15% | 64% | Nephropathy pos. |
| 23.3 ± 4.46 | 2045.9 ± 0.32 | 32% | 40% | Nephropathy pos. |
| 33.7 ± 3.16 | 2115.1 ± 0.53 | 30% | 40% | Nephropathy pos. |
| 20.5 ± 2.78 | 2177.1 ± 0.37 | 9% | 40% | Nephropathy pos. |
| 18.1 ± 4.24 | 2241.6 ± 0.41 | 9% | 59% | Nephropathy pos. |
| 21.2 ± 2.49 | 2250.7 ± 0.38 | 23% | 64% | Nephropathy pos. |
| 27.5 ± 2.53 | 2258.7 ± 0.49 | 9% | 59% | Nephropathy pos. |
| 20.0 ± 3.30 | 2356.4 ± 0.41 | 13% | 59% | Nephropathy pos. |
| 28.1 ± 3.95 | 2391.4 ± 0.42 | 13% | 64% | Nephropathy pos. |
| 25.7 ± 4.85 | 2406.1 ± 0.57 | 20% | 77% | Nephropathy pos. |
| 22.8 ± 4.28 | 2423.2 ± 0.53 | 14% | 64% | Nephropathy pos. |
| 21.9 ± 4.45 | 2427.3 ± 0.40 | 31% | 91% | Nephropathy pos. |
| 19.2 ± 4.24 | 2465.1 ± 0.62 | 9% | 77% | Nephropathy pos. |
| 25.4 ± 5.25 | 2493.0 ± 0.38 | 9% | 50% | Nephropathy pos. |
| 19.5 ± 4.66 | 2494.0 ± 0.66 | 12% | 77% | Nephropathy pos. |
| 23.7 ± 4.27 | 2494.9 ± 0.49 | 7% | 40% | Nephropathy pos. |
| 24.4 ± 5.51 | 2522.0 ± 0.67 | 17% | 82% | Nephropathy pos. |
| 20.1 ± 3.61 | 2540.5 ± 0.54 | 14% | 68% | Nephropathy pos. |
| 22.3 ± 4.72 | 2593.5 ± 0.30 | 7% | 55% | Nephropathy pos. |
| 20.0 ± 4.87 | 2613.9 ± 0.83 | 14% | 55% | Nephropathy pos. |
| 35.1 ± 1.62 | 2726.5 ± 0.67 | 61% | 20% | Nephropathy pos. |
| 25.0 ± 4.39 | 2775.1 ± 0.56 | 12% | 40% | Nephropathy pos. |
| 21.8 ± 3.78 | 2790.7 ± 0.55 | 19% | 86% | Nephropathy pos. |
| 25.9 ± 3.30 | 2892.2 ± 0.50 | 9% | 50% | Nephropathy pos. |
| 16.8 ± 2.72 | 2919.0 ± 0.26 | 2% | 50% | Nephropathy pos. |
| 21.9 ± 3.23 | 2937.0 ± 0.49 | 13% | 86% | Nephropathy pos. |
| 20.0 ± 4.81 | 2958.8 ± 0.80 | 5% | 59% | Nephropathy pos. |
| 34.4 ± 2.72 | 2962.0 ± 0.54 | 12% | 20% | Nephropathy pos. |
| 28.9 ± 3.56 | 3059.7 ± 0.78 | 30% | 40% | Nephropathy pos. |
| 28.3 ± 5.96 | 3088.0 ± 0.79 | 7% | 20% | Nephropathy pos. |
| 26.1 ± 2.72 | 3369.2 ± 0.73 | 21% | 40% | Nephropathy pos. |
| 26.0 ± 2.89 | 3483.4 ± 0.95 | 30% | 40% | Nephropathy pos. |
| 24.5 ± 3.92 | 4183.3 ± 1.44 | 4% | 40% | Nephropathy pos. |
| 21.0 ± 5.35 | 4241.0 ± 0.62 | 29% | 73% | Nephropathy pos. |
| 23.4 ± 4.09 | 4370.2 ± 1.01 | 11% | 40% | Nephropathy pos. |
| 22.8 ± 2.94 | 4527.6 ± 0.67 | 1% | 45% | Nephropathy pos. |
| 21.7 ± 3.00 | 4713.6 ± 0.44 | 7% | 64% | Nephropathy pos. |
| 24.6 ± 3.73 | 7556.6 ± 1.55 | 2% | 40% | Nephropathy pos. |
| 16.7 ± 5.54 | 8055.1 ± 2.10 | 12% | 40% | Nephropathy pos. |
| 13.2 ± 5.19 | 8765.8 ± 0.96 | 37% | 82% | Nephropathy pos. |
| 15.3 ± 4.97 | 9181.0 ± 1.28 | 10% | 64% | Nephropathy pos. |
| 14.0 ± 4.20 | 10046.1 ± 0.96 | 21% | 77% | Nephropathy pos. |
| 18.7 ± 5.50 | 10208.0 ± 1.24 | 2% | 40% | Nephropathy pos. |
| 17.4 ± 4.02 | 10518.2 ± 1.10 | 23% | 64% | Nephropathy pos. |
| 35.3 ± 5.04 | 924.5 ± 0.12 | 50% | 0% | Nephropathy neg. |
| 43.1 ± 2.61 | 928.4 ± 0.08 | 65% | 14% | Nephropathy neg. |
| 45.7 ± 2.25 | 955.5 ± 0.14 | 60% | 5% | Nephropathy neg. |
| 23.8 ± 2.94 | 1010.6 ± 0.09 | 67% | 5% | Nephropathy neg. |
| 31.2 ± 1.53 | 1028.5 ± 0.09 | 84% | 32% | Nephropathy neg. |
| 45.9 ± 2.27 | 1041.4 ± 0.10 | 57% | 0% | Nephropathy neg. |
| 31.5 ± 1.98 | 1046.5 ± 0.09 | 87% | 32% | Nephropathy neg. |
| 43.4 ± 2.24 | 1047.5 ± 0.12 | 68% | 0% | Nephropathy neg. |
| 18.1 ± 4.34 | 1050.7 ± 0.12 | 60% | 0% | Nephropathy neg. |
| 32.9 ± 3.03 | 1084.4 ± 0.11 | 69% | 18% | Nephropathy neg. |
| 46.7 ± 2.63 | 1125.5 ± 0.12 | 63% | 9% | Nephropathy neg. |
| 46.3 ± 2.70 | 1157.5 ± 0.10 | 83% | 32% | Nephropathy neg. |
| 43.7 ± 1.70 | 1160.5 ± 0.07 | 72% | 18% | Nephropathy neg. |
| 44.5 ± 3.67 | 1179.5 ± 0.09 | 97% | 36% | Nephropathy neg. |
| 45.0 ± 2.24 | 1191.6 ± 0.09 | 60% | 9% | Nephropathy neg. |
| 46.2 ± 2.59 | 1195.5 ± 0.10 | 98% | 32% | Nephropathy neg. |
| 44.2 ± 1.83 | 1200.6 ± 0.13 | 86% | 0% | Nephropathy neg. |
| 45.9 ± 2.04 | 1223.5 ± 0.10 | 80% | 9% | Nephropathy neg. |
| 44.5 ± 2.15 | 1239.6 ± 0.08 | 89% | 0% | Nephropathy neg. |
| 47.8 ± 3.08 | 1246.6 ± 0.11 | 60% | 5% | Nephropathy neg. |
| 46.8 ± 2.20 | 1254.7 ± 0.19 | 56% | 5% | Nephropathy neg. |
| 43.2 ± 2.90 | 1261.5 ± 0.16 | 91% | 36% | Nephropathy neg. |
| 48.6 ± 2.90 | 1262.5 ± 0.09 | 65% | 0% | Nephropathy neg. |
| 43.9 ± 2.16 | 1277.6 ± 0.11 | 67% | 0% | Nephropathy neg. |
| 36.7 ± 3.04 | 1288.7 ± 0.18 | 72% | 23% | Nephropathy neg. |
| 47.2 ± 3.17 | 1292.5 ± 0.14 | 67% | 18% | Nephropathy neg. |
| 47.8 ± 2.58 | 1308.5 ± 0.09 | 66% | 0% | Nephropathy neg. |
| 48.2 ± 2.67 | 1321.6 ± 0.11 | 53% | 0% | Nephropathy neg. |
| 34.8 ± 1.81 | 1321.7 ± 0.23 | 98% | 41% | Nephropathy neg. |
| 46.0 ± 4.93 | 1351.7 ± 0.15 | 63% | 9% | Nephropathy neg. |
| 47.7 ± 2.99 | 1367.6 ± 0.14 | 97% | 23% | Nephropathy neg. |
| 37.8 ± 2.93 | 1378.6 ± 0.16 | 87% | 36% | Nephropathy neg. |
| 47.5 ± 2.59 | 1389.7 ± 0.15 | 86% | 18% | Nephropathy neg. |
| 46.5 ± 2.28 | 1407.8 ± 0.20 | 79% | 9% | Nephropathy neg. |
| 44.6 ± 4.84 | 1422.1 ± 0.33 | 70% | 0% | Nephropathy neg. |
| 45.4 ± 3.62 | 1423.8 ± 0.19 | 75% | 0% | Nephropathy neg. |
| 48.0 ± 2.97 | 1424.7 ± 0.16 | 95% | 18% | Nephropathy neg. |
| 47.6 ± 3.40 | 1446.7 ± 0.16 | 92% | 23% | Nephropathy neg. |
| 46.5 ± 2.95 | 1450.4 ± 0.25 | 62% | 9% | Nephropathy neg. |
| 48.0 ± 2.95 | 1462.6 ± 0.17 | 97% | 9% | Nephropathy neg. |
| 35.7 ± 1.90 | 1487.7 ± 0.15 | 70% | 18% | Nephropathy neg. |
| 47.8 ± 2.35 | 1490.6 ± 0.12 | 72% | 9% | Nephropathy neg. |
| 49.2 ± 2.77 | 1491.7 ± 0.12 | 81% | 14% | Nephropathy neg. |
| 49.0 ± 3.14 | 1507.8 ± 0.17 | 99% | 32% | Nephropathy neg. |
| 49.2 ± 2.86 | 1523.7 ± 0.11 | 97% | 18% | Nephropathy neg. |
| 48.6 ± 2.70 | 1529.7 ± 0.19 | 83% | 9% | Nephropathy neg. |
| 49.2 ± 3.26 | 1539.7 ± 0.19 | 98% | 23% | Nephropathy neg. |
| 49.0 ± 3.19 | 1545.7 ± 0.13 | 99% | 23% | Nephropathy neg. |
| 49.8 ± 2.76 | 1561.6 ± 0.19 | 90% | 18% | Nephropathy neg. |
| 48.4 ± 3.12 | 1567.7 ± 0.20 | 65% | 9% | Nephropathy neg. |
| 48.1 ± 2.66 | 1573.7 ± 0.27 | 63% | 5% | Nephropathy neg. |
| 48.5 ± 4.03 | 1577.8 ± 0.35 | 94% | 9% | Nephropathy neg. |
| 50.6 ± 3.40 | 1587.1 ± 0.34 | 65% | 0% | Nephropathy neg. |
| 48.6 ± 2.68 | 1589.7 ± 0.14 | 86% | 18% | Nephropathy neg. |
| 45.9 ± 3.83 | 1591.7 ± 0.30 | 79% | 18% | Nephropathy neg. |
| 49.3 ± 3.22 | 1594.8 ± 0.14 | 88% | 14% | Nephropathy neg. |
| 48.8 ± 2.78 | 1605.7 ± 0.13 | 73% | 18% | Nephropathy neg. |
| 48.5 ± 2.81 | 1611.7 ± 0.14 | 73% | 5% | Nephropathy neg. |
| 46.3 ± 5.12 | 1636.4 ± 0.39 | 79% | 23% | Nephropathy neg. |
| 49.5 ± 3.37 | 1651.8 ± 0.19 | 99% | 23% | Nephropathy neg. |
| 45.2 ± 5.96 | 1657.7 ± 0.23 | 60% | 5% | Nephropathy neg. |
| 49.5 ± 3.33 | 1673.8 ± 0.14 | 95% | 23% | Nephropathy neg. |
| 49.6 ± 3.05 | 1689.8 ± 0.18 | 86% | 0% | Nephropathy neg. |
| 26.9 ± 3.18 | 1706.8 ± 0.30 | 78% | 27% | Nephropathy neg. |
| 49.4 ± 2.84 | 1734.4 ± 0.40 | 65% | 5% | Nephropathy neg. |
| 49.2 ± 3.17 | 1739.7 ± 0.22 | 59% | 5% | Nephropathy neg. |
| 45.1 ± 4.21 | 1748.0 ± 0.28 | 55% | 5% | Nephropathy neg. |
| 44.2 ± 4.71 | 1813.6 ± 0.38 | 58% | 5% | Nephropathy neg. |
| 39.1 ± 3.48 | 1817.0 ± 0.29 | 85% | 18% | Nephropathy neg. |
| 51.7 ± 3.48 | 1841.0 ± 0.23 | 59% | 9% | Nephropathy neg. |
| 50.4 ± 4.56 | 1848.2 ± 0.43 | 58% | 0% | Nephropathy neg. |
| 51.5 ± 2.94 | 1856.8 ± 0.24 | 59% | 5% | Nephropathy neg. |
| 52.7 ± 4.24 | 1863.8 ± 0.31 | 88% | 14% | Nephropathy neg. |
| 52.7 ± 3.92 | 1885.8 ± 0.20 | 70% | 5% | Nephropathy neg. |
| 47.7 ± 4.69 | 1902.1 ± 0.33 | 75% | 0% | Nephropathy neg. |
| 50.6 ± 3.95 | 1924.0 ± 0.48 | 68% | 0% | Nephropathy neg. |
| 26.6 ± 1.76 | 2048.5 ± 0.44 | 86% | 20% | Nephropathy neg. |
| 25.8 ± 1.39 | 2085.9 ± 0.24 | 83% | 32% | Nephropathy neg. |
| 39.9 ± 1.45 | 2087.8 ± 0.34 | 72% | 23% | Nephropathy neg. |
| 52.8 ± 4.09 | 2117.1 ± 0.17 | 78% | 9% | Nephropathy neg. |
| 28.3 ± 3.90 | 2129.7 ± 0.42 | 63% | 0% | Nephropathy neg. |
| 40.4 ± 1.53 | 2158.9 ± 0.26 | 86% | 32% | Nephropathy neg. |
| 39.7 ± 1.71 | 2174.9 ± 0.36 | 97% | 45% | Nephropathy neg. |
| 32.6 ± 1.79 | 2227.1 ± 0.41 | 81% | 23% | Nephropathy neg. |
| 29.3 ± 3.50 | 2249.0 ± 0.41 | 92% | 41% | Nephropathy neg. |
| 40.6 ± 1.25 | 2257.1 ± 0.35 | 94% | 45% | Nephropathy neg. |
| 46.2 ± 5.11 | 2273.5 ± 0.38 | 71% | 18% | Nephropathy neg. |
| 40.8 ± 2.66 | 2296.0 ± 0.40 | 63% | 20% | Nephropathy neg. |
| 40.9 ± 3.32 | 2327.6 ± 0.52 | 85% | 36% | Nephropathy neg. |
| 41.8 ± 2.45 | 2343.3 ± 0.43 | 77% | 27% | Nephropathy neg. |
| 40.8 ± 1.31 | 2385.3 ± 0.32 | 95% | 45% | Nephropathy neg. |
| 40.9 ± 2.68 | 2471.5 ± 0.52 | 69% | 14% | Nephropathy neg. |
| 41.5 ± 2.64 | 2493.5 ± 0.48 | 74% | 18% | Nephropathy neg. |
| 52.9 ± 3.98 | 2570.4 ± 0.27 | 71% | 5% | Nephropathy neg. |
| 34.1 ± 0.72 | 2642.8 ± 0.40 | 86% | 36% | Nephropathy neg. |
| 36.1 ± 2.56 | 2687.1 ± 0.49 | 84% | 23% | Nephropathy neg. |
| 42.8 ± 2.33 | 2710.6 ± 0.46 | 88% | 18% | Nephropathy neg. |
| 50.6 ± 4.73 | 2748.6 ± 0.36 | 64% | 0% | Nephropathy neg. |
| 37.8 ± 1.92 | 2986.6 ± 0.55 | 74% | 23% | Nephropathy neg. |
| 23.3 ± 2.07 | 3007.4 ± 0.50 | 65% | 9% | Nephropathy neg. |
| 25.9 ± 2.35 | 3038.3 ± 0.70 | 46% | 0% | Nephropathy neg. |
| 46.0 ± 2.91 | 3045.4 ± 0.36 | 59% | 5% | Nephropathy neg. |
| 53.3 ± 4.05 | 3057.2 ± 0.64 | 76% | 9% | Nephropathy neg. |
| 38.9 ± 2.57 | 3109.0 ± 0.57 | 88% | 14% | Nephropathy neg. |
| 41.9 ± 3.55 | 3187.6 ± 0.47 | 71% | 14% | Nephropathy neg. |
| 26.6 ± 1.15 | 3193.6 ± 0.41 | 61% | 0% | Nephropathy neg. |
| 48.3 ± 3.69 | 3223.8 ± 0.41 | 88% | 18% | Nephropathy neg. |
| 31.7 ± 3.65 | 3265.1 ± 0.64 | 93% | 41% | Nephropathy neg. |
| 29.5 ± 1.76 | 3291.0 ± 0.52 | 81% | 23% | Nephropathy neg. |
| 49.2 ± 3.70 | 3293.1 ± 0.43 | 91% | 14% | Nephropathy neg. |
| 49.9 ± 3.57 | 3315.0 ± 0.45 | 67% | 5% | Nephropathy neg. |
| 43.3 ± 2.04 | 3319.9 ± 0.66 | 86% | 23% | Nephropathy neg. |
| 49.1 ± 3.35 | 3336.7 ± 0.38 | 63% | 9% | Nephropathy neg. |
| 38.5 ± 2.05 | 3359.9 ± 0.42 | 98% | 41% | Nephropathy neg. |
| 38.5 ± 1.92 | 3360.1 ± 0.65 | 98% | 20% | Nephropathy neg. |
| 38.5 ± 2.03 | 3417.1 ± 0.48 | 95% | 45% | Nephropathy neg. |
| 38.5 ± 1.09 | 3433.3 ± 0.43 | 92% | 41% | Nephropathy neg. |
| 51.6 ± 3.50 | 3478.9 ± 0.48 | 74% | 5% | Nephropathy neg. |
| 31.7 ± 2.29 | 3589.7 ± 0.48 | 73% | 18% | Nephropathy neg. |
| 33.2 ± 3.71 | 3633.4 ± 0.95 | 80% | 18% | Nephropathy neg. |
| 36.0 ± 3.18 | 3636.6 ± 0.73 | 58% | 0% | Nephropathy neg. |
| 37.9 ± 2.69 | 3719.5 ± 0.61 | 67% | 9% | Nephropathy neg. |
| 42.0 ± 3.21 | 3739.7 ± 0.99 | 73% | 14% | Nephropathy neg. |
| 25.8 ± 1.20 | 3947.3 ± 0.67 | 92% | 32% | Nephropathy neg. |
| 39.4 ± 1.13 | 4006.6 ± 0.49 | 62% | 5% | Nephropathy neg. |
| 26.0 ± 3.97 | 4044.9 ± 0.56 | 78% | 14% | Nephropathy neg. |
| 30.5 ± 2.17 | 4070.4 ± 0.48 | 57% | 5% | Nephropathy neg. |
| 29.5 ± 0.93 | 4098.6 ± 0.52 | 86% | 32% | Nephropathy neg. |
| 34.3 ± 2.08 | 4102.5 ± 0.50 | 77% | 14% | Nephropathy neg. |
| 34.7 ± 0.63 | 4290.7 ± 0.52 | 76% | 18% | Nephropathy neg. |
| 23.5 ± 1.61 | 4405.8 ± 0.54 | 51% | 0% | Nephropathy neg. |
| 30.4 ± 1.31 | 4801.5 ± 1.06 | 65% | 0% | Nephropathy neg. |
| 32.4 ± 1.31 | 4863.8 ± 0.64 | 67% | 5% | Nephropathy neg. |
| 29.5 ± 2.25 | 5214.0 ± 1.29 | 51% | 0% | Nephropathy neg. |
| 33.0 ± 0.99 | 6172.0 ± 1.57 | 65% | 0% | Nephropathy neg. |
| 33.2 ± 0.75 | 6187.8 ± 0.75 | 95% | 45% | Nephropathy neg. |
| 23.8 ± 1.86 | 9869.7 ± 1.06 | 69% | 14% | Nephropathy neg. |

TABLE 23

| migration time [min] | dt [min] | mass [Da] |
|---|---|---|
| 15.490396 | 0.158804 | 8054.473633 |
| 15.803237 | 0.155143 | 8765.233398 |
| 16.034266 | 0.174906 | 1621.9104 |
| 16.185061 | 0.147871 | 9180.99707 |
| 16.645294 | 0.198704 | 10045.20703 |
| 17.663696 | 0.165531 | 10388.81348 |
| 17.980883 | 0.178564 | 10518.18457 |
| 19.917442 | 0.234131 | 9220.939453 |
| 20.34516 | 0.170572 | 1877.789429 |
| 20.479975 | 0.221246 | 3842.693604 |
| 20.519386 | 0.265078 | 4747.932617 |
| 21.465685 | 0.217493 | 4154.003906 |
| 21.480436 | 0.362197 | 2427.251709 |
| 21.804012 | 0.271715 | 4240.856445 |
| 22.221563 | 0.191069 | 4282.796387 |
| 22.777784 | 0.245503 | 3840.540527 |
| 24.304148 | 0.319715 | 7556.177734 |
| 24.579231 | 0.291986 | 879.519653 |
| 24.813087 | 0.224198 | 1867.731689 |
| 25.283239 | 0.22054 | 2266.040771 |
| 26.177101 | 0.289898 | 2172.188721 |
| 26.773794 | 0.352887 | 2914.05542 |
| 26.81407 | 0.297343 | 962.591919 |
| 28.254925 | 0.581783 | 4353.585938 |
| 28.825331 | 0.258778 | 1250.62439 |
| 29.308136 | 0.852391 | 1060.239014 |
| 29.822325 | 0.595913 | 1682.720947 |
| 30.75272 | 0.175961 | 943.492859 |
| 30.762201 | 0.263861 | 1108.647949 |
| 30.926645 | 0.138075 | 1368.781738 |
| 31.305229 | 0.301605 | 3987.548828 |
| 31.433071 | 0.515308 | 1099.419434 |
| 32.165497 | 0.198377 | 3122.730713 |
| 32.222111 | 0.226858 | 1829.089966 |
| 33.427856 | 0.151562 | 2767.015625 |
| 34.053886 | 0.252424 | 1302.691772 |
| 34.15913 | 0.233032 | 3722.875977 |
| 34.557327 | 0.186137 | 2039.143433 |
| 34.681156 | 0.20976 | 3685.918213 |
| 35.30254 | 0.207782 | 2389.097168 |
| 35.502213 | 0.388916 | 3209.800293 |
| 36.314056 | 0.183495 | 980.526123 |
| 36.404907 | 0.145751 | 1008.513733 |
| 36.424831 | 0.150486 | 1000.48761 |
| 36.720509 | 0.128397 | 2717.472656 |
| 36.777012 | 0.164648 | 2663.246826 |
| 37.557594 | 0.165628 | 3556.580566 |
| 37.572525 | 0.185484 | 1743.890381 |
| 37.680653 | 0.160958 | 1134.580566 |
| 37.700241 | 0.171622 | 4097.981934 |
| 38.050472 | 0.156383 | 3152.361572 |
| 38.155159 | 0.217341 | 2825.309082 |
| 38.17057 | 0.432096 | 882.532654 |
| 38.281631 | 0.20781 | 996.190369 |
| 38.57658 | 0.370648 | 1425.324829 |
| 38.687305 | 0.15052 | 3385.513916 |
| 38.830559 | 0.056085 | 1352.824097 |
| 38.921108 | 0.150325 | 5000.982422 |
| 39.241917 | 0.178206 | 3775.720459 |
| 39.433277 | 0.235333 | 3405.60791 |
| 39.484215 | 0.140887 | 1046.52771 |
| 39.513248 | 0.093703 | 2154.053955 |
| 39.936756 | 0.195951 | 6171.129395 |
| 40.533363 | 0.158628 | 1194.581543 |
| 40.537457 | 0.221485 | 2205.064941 |
| 40.607231 | 0.426674 | 1235.384888 |
| 40.686531 | 0.122381 | 1265.634888 |
| 40.83009 | 0.191972 | 2642.264893 |
| 41.506096 | 0.161887 | 4159.304199 |
| 41.604115 | 0.217324 | 1250.585449 |
| 41.818069 | 0.163642 | 2742.253418 |
| 42.079609 | 0.266392 | 1463.643311 |
| 42.105633 | 0.172054 | 1489.658936 |
| 42.131275 | 0.184863 | 1473.643555 |
| 42.144161 | 0.162716 | 1451.710938 |
| 42.573879 | 0.234118 | 3098.450928 |
| 42.636433 | 0.041732 | 1487.660034 |
| 42.811199 | 0.246696 | 1579.670776 |
| 42.940624 | 0.1884 | 3121.243164 |
| 43.093792 | 0.106392 | 3271.523438 |
| 43.115334 | 0.607341 | 1834.878052 |
| 43.46143 | 0.193155 | 3442.135498 |
| 43.494144 | 0.20218 | 3495.841797 |
| 43.549488 | 0.217899 | 3473.905029 |
| 43.740391 | 0.12795 | 3108.919434 |
| 44.191006 | 0.18629 | 3359.583496 |
| 44.230297 | 0.233319 | 3416.526611 |
| 44.934914 | 0.127421 | 1991.917114 |
| 45.536418 | 0.214716 | 2197.337158 |
| 45.675098 | 0.12333 | 1889.864502 |
| 46.313114 | 0.259721 | 2385.597168 |
| 47.216648 | 0.168651 | 2649.602539 |
| 47.279705 | 0.127824 | 2343.072998 |
| 47.526871 | 0.19233 | 2584.635986 |
| 48.441795 | 0.239347 | 1160.526001 |
| 48.804813 | 0.251244 | 1261.53125 |
| 49.519478 | 0.243133 | 1274.625244 |
| 51.416531 | 0.33207 | 1195.518677 |
| 51.492035 | 0.213235 | 1211.559204 |
| 51.657627 | 0.822884 | 1223.348633 |
| 53.168346 | 0.293424 | 1351.643433 |
| 53.240913 | 0.216809 | 1367.655151 |
| 53.259499 | 0.15916 | 1770.30481 |
| 54.59832 | 0.234281 | 1507.742432 |
| 55.038143 | 0.329349 | 1594.211426 |
| 57.475471 | 0.325805 | 1840.810547 |
| 58.191887 | 0.129 | 2021.900879 |
| 58.898354 | 0.484288 | 2608.239746 |
| 60.082333 | 0.507699 | 1863.939453 |

TABLE 24

| migration time [min] | dt [min] | mass [Da] |
|---|---|---|
| 12.295616 | 0.092835 | 8053.516 |
| 12.331619 | 0.12201 | 1621.946 |
| 12.508785 | 0.139706 | 8765.729 |
| 12.696615 | 0.122507 | 9181.114 |
| 12.906662 | 0.115952 | 10046.58 |
| 13.103853 | 0.041984 | 2427.001 |
| 14.332394 | 0.144029 | 4153.814 |
| 14.426023 | 0.131007 | 4240.702 |
| 14.496774 | 0.385605 | 3841.615 |
| 14.585806 | 0.105399 | 4282.281 |
| 15.094264 | 0.132582 | 879.5324 |
| 15.123884 | 0.069059 | 1868.033 |
| 15.236325 | 0.136994 | 7555.679 |
| 15.641728 | 0.159929 | 962.6218 |
| 16.194395 | 0.167525 | 1060.664 |
| 16.280394 | 0.28676 | 4353.476 |
| 16.363562 | 0.082856 | 1682.889 |
| 16.427116 | 0.09725 | 1743.982 |
| 16.50071 | 0.133981 | 1108.646 |
| 16.904119 | 0.128321 | 1829.115 |
| 17.017418 | 0.23895 | 3987.366 |
| 17.409172 | 0.158398 | 2767.263 |
| 17.716999 | 0.1571 | 1302.722 |
| 17.891594 | 0.202698 | 3722.962 |
| 18.049681 | 0.191037 | 2039.257 |
| 18.140236 | 0.176836 | 3686.508 |
| 18.528196 | 0.076336 | 3209.884 |
| 19.106394 | 0.148381 | 1008.572 |
| 19.118612 | 0.156251 | 1000.564 |
| 19.173443 | 0.122128 | 980.5635 |
| 19.335644 | 0.098819 | 2663.262 |
| 19.367334 | 0.112794 | 2718.314 |
| 20.023649 | 0.199337 | 3556.408 |
| 20.041323 | 0.195353 | 1134.629 |
| 20.063593 | 0.224531 | 4098.26 |
| 20.300522 | 0.113124 | 3152.333 |
| 20.347666 | 0.200969 | 882.5596 |
| 20.470793 | 0.208903 | 2825.334 |
| 20.889994 | 0.26629 | 3385.819 |
| 20.93943 | 0.057322 | 1425.772 |
| 21.519066 | 0.226397 | 5000.98 |
| 21.655712 | 0.298068 | 3775.697 |
| 21.755213 | 0.316991 | 1046.586 |
| 21.850452 | 0.518151 | 3405.871 |
| 22.747589 | 0.302407 | 1235.601 |
| 22.763943 | 0.277557 | 1194.603 |
| 22.997269 | 0.34528 | 1265.661 |
| 23.013165 | 0.225846 | 2642.188 |
| 23.017294 | 0.551478 | 6171.03 |
| 23.838888 | 0.406385 | 1250.653 |
| 24.025209 | 0.261109 | 2742.267 |
| 24.137253 | 0.135523 | 1463.693 |
| 24.14039 | 0.158709 | 1473.664 |
| 24.220345 | 0.206216 | 1489.686 |
| 24.355286 | 0.409203 | 1451.684 |
| 24.686199 | 0.240303 | 3098.376 |
| 24.915867 | 0.332783 | 1579.718 |
| 25.093962 | 0.214003 | 3121.259 |
| 25.181305 | 0.33936 | 3272.276 |
| 25.634459 | 0.407648 | 3441.958 |
| 25.648405 | 0.344555 | 3495.801 |
| 25.928818 | 0.283113 | 3108.66 |
| 26.411203 | 0.355909 | 3359.75 |
| 26.493782 | 0.341234 | 3416.324 |
| 27.775286 | 0.346393 | 2196.686 |
| 28.415859 | 0.219954 | 2385.565 |
| 29.471397 | 0.2699 | 2649.791 |
| 29.74654 | 0.131224 | 2584.214 |
| 30.499264 | 0.32736 | 1160.556 |
| 30.832899 | 0.269278 | 1261.477 |
| 32.240211 | 0.415696 | 1195.543 |
| 32.240601 | 0.406316 | 1223.53 |
| 32.29216 | 0.268596 | 1212.024 |
| 33.24297 | 0.403599 | 1367.633 |
| 34.039223 | 0.467469 | 1507.75 |
| 34.274136 | 0.432896 | 1594.746 |
| 35.978645 | 0.326975 | 1841.202 |
| 37.237282 | 0.110906 | 2608.186 |
| 37.342949 | 0.6411 | 1863.833 |

TABLE 25

| | | | | | Immuno- |
|---|---|---|---|---|---|
| Sex | Age | Diagnosis | S-creatinine | Proteinuria | suppression |
| M | 63 | FSGS | 95 | 0.02 | PS |
| M | 18 | FSGS | 99 | 0.05 | CsA |
| M | 63 | FSGS | 93 | 0.05 | PS |
| F | 49 | FSGS | 80 | 0.05 | CsA + PS |
| F | 23 | FSGS | 69 | 0.54 | CsA |
| F | 26 | FSGS | 16 | 0.7 | CsA |
| F | 56 | FSGS | 80 | 0.8 | — |
| M | 62 | FSGS | 150 | 1.9 | — |
| M | 26 | FSGS | 144 | 4.9 | — |
| F | 26 | FSGS | 150 | 11.0 | CsA + PS |
| M | 69 | MGN | 128 | 0.02 | CsA |
| M | 62 | MGN | 91 | 0.17 | — |
| M | 23 | MGN | 150 | 0.3 | — |
| M | 37 | MGN | 73 | 0.33 | — |
| M | 43 | MGN | 82 | 0.7 | PS |
| M | 48 | MGN | 100 | 1.0 | CsA + PS |
| F | 68 | MGN | 150 | 1.0 | — |
| F | 21 | MGN | 80 | 1.0 | CsA + PS |
| M | 44 | MGN | 118 | 1.0 | CsA |
| M | 45 | MGN | 93 | 1.3 | — |
| M | 48 | MGN | 133 | 2.4 | — |
| M | 37 | MGN | 93 | 2.6 | — |
| M | 78 | MGN | 99 | 3.3 | — |
| M | 47 | MGN | 93 | 3.5 | PS |
| F | 34 | MGN | 80 | 3.5 | CsA + PS |
| M | 66 | MGN | 132 | 3.6 | — |
| M | 38 | MGN | 100 | 4.0 | CsA + PS |
| M | 43 | MGN | 85 | 5.1 | — |
| F | 43 | MCD | 114 | 0.01 | CsA |
| M | 45 | MCD+ | 93 | 0.01 | — |
| F | 52 | MCD+ | 118 | 0.01 | — |
| M | 52 | MCD | 93 | 0.01 | — |
| F | 44 | MCD+ | 80 | 0.02 | CsA |
| M | 39 | MCD* | 93 | 0.02 | — |
| M | 51 | MCD | 93 | 0.05 | — |
| M | 18 | MCD | 77 | 0.05 | CsA + PS |
| F | 70 | MCD* | 95 | 0.08 | — |
| M | 69 | MCD | 93 | 0.08 | — |
| F | 29 | MCD+ | 160 | 0.1 | — |
| M | 62 | MCD+ | 93 | 0.1 | — |
| M | 21 | MCD | 57 | 0.12 | — |
| F | 43 | MCD | 114 | 0.01 | CSA |
| F | 25 | MCD | 80 | 1.2 | — |
| M | 52 | MCD | 93 | 0.4 | PS |
| F | 80 | MCD* | 145 | 7.9 | — |

1. A diagnostic marker comprising a polypeptide marker recoverable from a urine sample for diagnosis of a renal disease, wherein the polypeptide marker is selected from the group of polypeptide markers as shown in tables 1 to 22.

2. The diagnostic marker of claim 1, wherein the renal disease is chosen from the group consisting of IgA-nephropathy, MGN, MCD, FSGS, and diabetic nephropathy.

3. The diagnostic marker of claim 1, wherein the renal disease is IgA-nephropathy.

4. The diagnostic marker of claim 1, wherein diagnosis relates to differential diagnosis between at least two diseases chosen from the group consisting of IgA-nephropathy, MGN, MCD, FSGS, and diabetic nephropathy.

5. The diagnostic marker of claim 1, wherein the polypeptide marker is selected from the group of polypeptide markers as shown in tables 1 to 13.

6. The diagnostic marker of claim 3, wherein the polypeptide marker is selected from the group of polypeptide markers as shown in tables 11 to 13.

7. A method for the diagnosis of a renal disease, the method comprising:
a) measuring the presence or the absence of a polypeptide marker in a urine sample, wherein the polypeptide marker is selected from the group of polypeptide markers shown in tables 1 to 22, and
b) comparing the probability of the presence of this marker in a disease patient to the probability of the presence of this marker in a control patient, wherein
c1) if the probability of the presence of this marker in a disease patient is higher than the probability of the presence of this marker in a control patient, the presence of this marker is indicative for a higher probability of having the disease rather than the control condition, or
c2) if the probability of the presence of this marker in a disease patient is lower than the probability of the presence of this marker in a control patient, the absence of the marker is indicative for a higher probability of having the disease rather than the control condition.

8. The method according to claim 7, wherein the individual probabilities in step b) are as indicated in the tables.

9. The method according to claim 7, wherein the control represents the healthy condition.

10. The method according to claim 7, wherein the control represents a renal disease, particularly chosen from the group consisting of IgA-nephropathy, MGN, MCD, FSGS, and diabetic nephropathy.

11. The method according to claim 7, wherein the polypeptide marker is selected from the group of polypeptide markers shown in tables 11 to 13.

12. The method according to claim 7, wherein the method comprises detecting a plurality of the polypeptide markers, preferably at least 3, more preferably at least 10, most preferably at least 50 of the polypeptide markers.

13. The method according to claim 7, wherein ELISA, quantitative Western Blot, radio-immuno-assay, surface plasmon resonance, array, gel electrophoresis, capillary electrophoresis, gas phase ion spectrometry, or mass spectrometry is used for detecting the presence of the marker or markers.

14. The method according to claim 7, wherein the polypeptide markers in the sample are separated by capillary electrophoresis before measurement.

15. The method according to claim 14, wherein mass spectrometry is used for detecting the presence of the marker or markers.

16. A method of diagnosing a patient comprising performing capillary electrophoresis-mass spectrometry on a sample from the patient to differentially diagnose a renal disease in vitro.

17. The method according to claim 16, wherein the renal disease is selected from the group consisting of IgA-nephropathy, MGN, MCD, FSGS, and diabetic nephropathy.

18. A diagnostic method comprising using capillary electrophoresis-mass spectrometry to differentially diagnose between at least two renal diseases selected from the group consisting of IgA-nephropathy, MGN, MCD, FSGS, and diabetic nephropathy.


SEQUENCE LISTING (5 sequences)

SEQ ID NO: 1 - protein, 79 aa, Homo sapiens
FTFHADICTLSEKERQIKKQTALVELVKHKPKATKEQLKAVMDDFAAFVEKCCKADDKETCFAEEGKKLVAASQAALGL

SEQ ID NO: 2 - protein, 90 aa, Homo sapiens
TYVPKEFNAETFTFHADICTLSEKERQIKKQTALVELVKHKPKATKEQLKAVMDDFAAFVEKCCKADDKETCFAEEGKKLVAASQAALGL

SEQ ID NO: 3 - protein, 9 aa, Homo sapiens
GGRPSRPPQ

SEQ ID NO: 4 - protein, 11 aa, Homo sapiens
GFRHRHPDEAA

SEQ ID NO: 5 - protein, 14 aa, Homo sapiens
GLITLIGINPSLHT